(19) United States
(12) Patent Application Publication
(10) Pub. No.: US 2008/0269233 A1
(43) Pub. Date: Oct. 30, 2008

(54) Piperidinoyl-Pyrrolidine and Piperidinoyl-Piperidine Compounds

(76) Inventors: Mark David Andrews, Kent (GB); Alan Daniel Brown, Kent (GB); David Sebastien, Fradet (GB); Mark Ian Lansdell, Kent (GB)

Correspondence Address: PFIZER INC., PATENT DEPARTMENT, MS8260-1611, EASTERN POINT ROAD, GROTON, CT, 06340, US

(21) Appl. No.: 11/997,824

(22) PCT Filed: Jul. 26, 2006
(86) PCT No.: PCT/IB06/02151

§ 371 Date: Feb. 28, 2008

(51) Int. Cl. A61K 31/501; C07D 401/06; A61K 31/435; A61P 3/04; A61P 15/10; C07D 403/14

(52) U.S. Cl. 514/252.03; 546/208; 514/326; 544/238

(57) ABSTRACT

The present invention relates to a class of compounds of general formula (I)

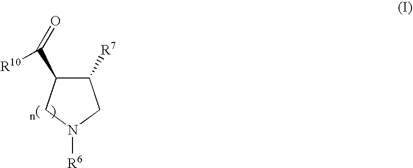

and the salts, hydrates, solvates, polymorphs and prodrugs wherein n, R⁶, R⁷ and R¹⁰ are as defined herein and especially to MCR4 agonist compounds of formula (I), to their use in medicine, particularly in the treatment of sexual dysfunction and obesity, to intermediates useful in their synthesis and to compositions containing them.

The present invention relates to a novel class of piperidinoyl-pyrrolidine and piperidinoyl-piperidine compounds, especially melanocortin receptor 4 (MCR4) agonist piperidinoyl-pyrrolidine and piperidinoyl-piperidine compounds and especially to selective MCR4 agonist piperidinoyl-pyrrolidine and piperidinoyl-piperidine compounds, to their use in medicine, to compositions containing them, to processes for their preparation and to intermediates used in such processes. In particular the present invention relates to a class of piperidinoyl-pyrrolidine and piperidinoyl-piperidine compounds which may be useful in the treatment of conditions which would benefit from agonism at the MCR4 receptor, such as sexual dysfunctions and/or obesity.

Compounds of the present invention may be useful in treating diseases, disorders or conditions responsive to activation of the MC4 receptor, including:

male and female sexual dysfunctions including hypoactive sexual desire disorder, sexual arousal disorder, orgasmic disorder and/or sexual pain disorder in females, male erectile dysfunction;
obesity (by reducing appetite, increasing metabolic rate, reducing fat intake or reducing carbohydrate craving); and
diabetes mellitus (by enhancing glucose tolerance and/or decreasing insulin resistance).

The compounds of the invention are potentially useful in treating further diseases, disorders or conditions including, but not limited to, hypertension, hyperlipidemia, osteoarthritis, cancer, gall bladder disease, sleep apnea, depression, anxiety, compulsion, neuroses, insomnia/sleep disorder, substance abuse, pain, fever, inflammation, immune modulation, rheumatoid arthritis, skin tanning, acne and other skin disorders, neuroprotective and cognitive and memory enhancement including the treatment of Alzheimer's disease, treatment of Lower Urinary Tract Dysfunction conditions (e.g.

(i) urinary incontinence, including stress urinary incontinence, urge urinary incontinence and mixed urinary incontinence;
(ii) overactive bladder (OAB), which includes the symptoms of increased daytime frequency and 5 urgency, and nocturia, which symptoms may or may not result in loss of urine (OAB wet and OAB dry), and urge incontinence; and
(iii) lower urinary tract symptoms (LUTS) comprising all the above symptoms, and, when associated with BPH, the additional symptoms of terminal dribble, hesitancy, intermittency, straining and poor flow.)

Compounds of the present invention are thought to be particularly suitable for treating female sexual dysfunction, male erectile dysfunction, obesity, diabetes, and conditions of Lower Urinary Tract Dysfunction.

Desirable properties for the MCR4 agonist compounds of the present invention include: desirable MCR4 potencies as detailed hereinafter; selectivity for MCR4 versus MCR1, and/or MCR5, and/or MCR3 as detailed hereinafter; both desirable MC4R potency and selectivity for MCR4 versus, MCR1, and/or MCR5, and/or MCR3; good biopharmaceutical properties such as physical stability; solubility; appropriate metabolic stability.

The present invention provides for compounds of formula (I):

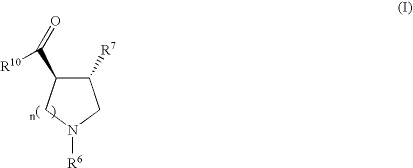

wherein:
n is 1 or 2;
R⁶ is selected from H, C₁-C₆alkyl, C₃-C₈cycloalkyl, aryl, heterocyclyl, heteroaryl, C(O)C₁-C₆alkyl, CO₂C₁-C₆alkyl, wherein each of said moieties is optionally substituted with one or more substituents independently selected from halo, CN, OH, ═O, NH₂, NHCH₃, N(CH₃)₂, C₁-C₄alkyl and C₁-C₄alkoxy;
R¹ is selected from pyridinyl and phenyl, wherein said pyridinyl or said phenyl is optionally substituted by 1-3 groups independently selected from halo, CN, CF₃, OCF₃, OC₁-C₄alkyl and C₁-C₄alkyl;
R¹⁰ is a substituted piperidine group of formula (II):

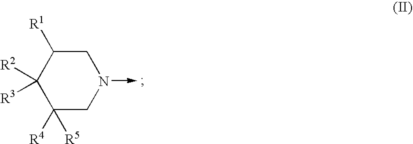

wherein
R¹ and R⁴ are each independently selected from H, C₁-C₄alkyl, OH, O(C₁-C₄alkyl), CH₂OCH₃ and NR⁸R⁹;
R² is selected from H, OH, OC₁-C₄alkyl and NR⁸R⁹;
R³ is selected from aryl or heteroaryl, wherein said moieties are optionally substituted with one or more substituents independently selected from halo, CN, CF₃, OCF₃, O(C₁-C₄alkyl), and C₁-C₄alkyl;
R⁵ is selected from H and C₁-C₄alkyl;
R⁸ is selected from H and C₁-C₄alkyl, wherein said C₁-C₄alkyl is optionally substituted with OH or OCH₃;
R⁹ is selected from H, C₁-C₄alkyl, SO₂C₁-C₄alkyl, C(O)C₁-C₄alkyl;
   wherein aryl means a six or ten membered aromatic hydrocarbon ring which is optionally fused to another six or ten membered aromatic hydrocarbon ring;
   wherein heteroaryl means a 5 or 6 membered aromatic ring, containing from 1 to 4 heteroatoms, said heteroatoms each independently selected from O, S and N, wherein said aromatic ring may be optionally fused to an aryl or second, non-fused, aromatic heterocyclic ring;
   wherein heterocyclyl means a 4 to 7 membered saturated or partially saturated ring, containing from 1 to 2 heteroatoms each independently selected from O, S and N;
   wherein halo means Cl, F, Br or I;
   and pharmaceutically acceptable salts, hydrate, solvates, polymorphs and prodrugs thereof, with the provisos that:
   R¹, R⁴ and R⁵ are not all simultaneously H;
   when R¹ is methyl and R⁴ is H, then R⁵ is not methyl;
   when R⁴ is methyl and R⁵ is H, then R¹ is not methyl; and
   when R⁵ is methyl and R⁴ is H, then R¹ is not methyl.

Alkyl is straight chain or branched.

Suitable aryl groups include phenyl and naphthyl.

Suitable heteroaryl groups include pyridinyl, pyrimidinyl, pyridazinyl and pyrazinyl, pyrrolyl, furanyl, pyrazolyl, imidazolyl, oxazolyl, isoxazolyl, thiazolyl, tetrazolyl, 1,2,3-triazolyl, 1,3,4-triazolyl, indolyl, indazolyl, pyrrolopyridinyl, pyrrolopyrimidinyl, benzimidazolyl, isoquinolinyl and quinolinyl.

Suitable heterocyclyl groups include 6-oxo-1,6-dihydropyridazin-3-yl, 6-oxo-1,6-dihydropyridin-3-yl, 2-oxo-1,2-dihydropyrimidine-4-yl, 2-oxo-1,2-dihydropyridin-4-yl, 6-oxo-1,6-dihydropyridin-2-yl, azetidinyl, tetrahydrofuranyl, pyrrolidinyl, tetrahydropyranyl, piperidinyl, piperazinyl, dihydropyranyl and tetrahydropyridinyl.

Unless otherwise indicated, the term substituted means substituted by one or more defined groups. In the case where groups may be selected from a number of alternatives groups, the selected groups may be the same or different.

Compounds of formula (I) contain two or more asymmetric carbon atoms and therefore exist in different stereoisomeric forms. Furthermore, the skilled person will understand that the present invention encompasses all stereoisomeric and diastereoisomeric forms, in particular compounds of general formula (IA), (IB), (IC), (ID), (IE), (IF), (IG) and (IH):

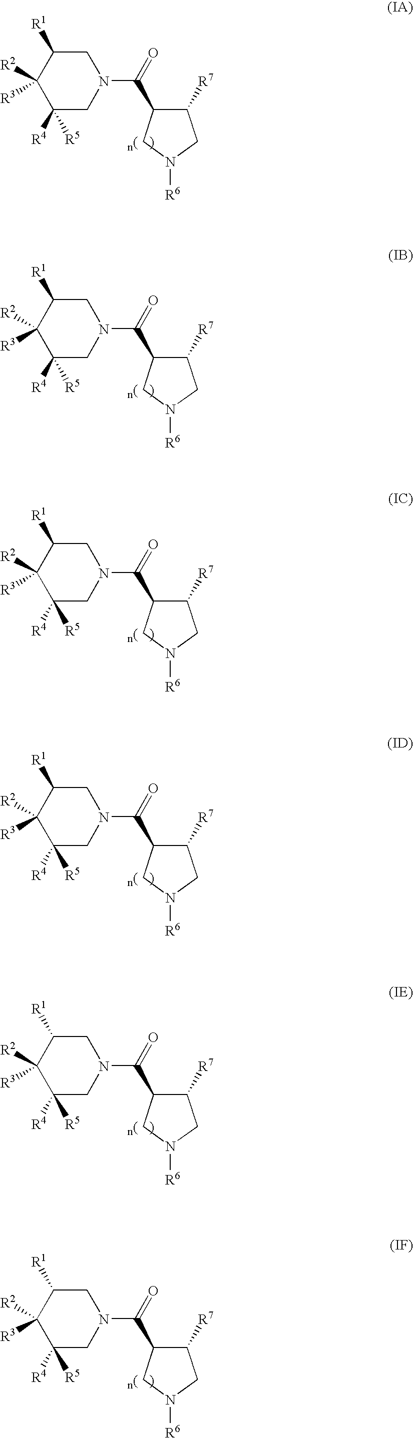

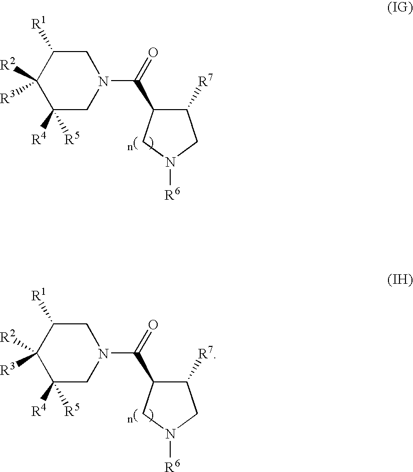

Separation of diastereoisomers may be achieved by conventional techniques, e.g. by fractional crystallisation, chromatography or H.P.L.C. of a stereoisomeric mixture of a compound of formula (IA), (IB), (IC), (ID), (IE), (IF), (IG) or (IH) or a suitable salt or derivative thereof. An individual enantiomer of a compound of formula (IA), (IB), (IC), (ID), (IE), (IF), (IG) or (IH) may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

In a first aspect, particularly preferred embodiments of the invention are as follows:
Preferably n is 1.
Preferably R¹ is selected from H, methyl, OH, OCH₃, OC₂H₅ and NR⁸R⁹.
More preferably R¹ is selected from H, methyl, OH, OCH₃ and OC₂H₅.
More preferably still R¹ is selected from H, methyl, OH and OCH₃.
Most preferably R¹ is selected from H, methyl and OCH₃.
Preferably R² is selected from H, OH, and OC₁-C₄alkyl.
More preferably R² is selected from H, OH, OCH₃ and OC₂H₅.
Most preferably R² is selected from OH, OCH₃ and OC₂H₅.
Preferably R³ is selected from aryl or heteroaryl, wherein said moieties are optionally substituted with one or more substituents independently selected from halo, CN, CF₃, OCF₃, OCH₃, OC₂H₅, methyl and ethyl.
More preferably R³ is selected from phenyl or heteroaryl, wherein said moieties are optionally substituted with one or more substituents independently selected from Cl, F, CN, CF₃, OCF₃, OCH₃, and methyl.
More preferably still R³ is selected from phenyl or pyridinyl, wherein said moieties are optionally substituted with one or more substituents independently selected from F, Cl, CN, OCH₃ and CF₃. Most preferably R³ is selected from phenyl and 2-pyridinyl, wherein said phenyl or 2-pyridinyl is optionally substituted with one or more substituents independently selected from F and Cl.
Preferably R⁴ is selected from H, methyl, OH, OCH₃, OC₂H₅ and NR⁸R⁹.
More preferably R⁴ is selected from H, methyl, OH, OCH₃ and OC₂H₅.
More preferably still R⁴ is selected from H, methyl, OCH₃ and OC₂H₅.
Most preferably R⁴ is selected from H, methyl and OCH₃.
Preferably R⁵ is selected from H, methyl and ethyl.
More preferably R⁵ is selected from H and methyl.
Most preferably R⁵ is H.
Preferably R⁶ is selected from C₁-C₈alkyl, C₃-C₈cycloalkyl, heterocyclyl, heteroaryl, C(O)C₁-C₆alkyl, CO₂C₁-C₆alkyl, wherein each of said moieties are optionally substituted with one or more substituents independently selected from halo, CN, OH, ═O, C₁-C₄alkyl and C₁-C₄alkoxy. More preferably R⁶ is selected from C₁-C₆alkyl, heterocyclyl, heteroaryl, C(O)C₁-C₆alkyl, CO₂C₁-C₆alkyl, wherein each of said moieties are optionally substituted with one or more substituents independently selected from halo, CN, OH, ═O, C₁-C₄alkyl and C₁-C₄alkoxy.
More preferably still R⁶ is selected from C₁-C₄alkyl, tetrahydropyranyl, tetrahydrofuranyl, pyrimidinyl, pyridinyl and pyridazinyl, wherein each of said moieties are optionally substituted with one or more substituents independently selected from halo, CN, OH, ═O, methyl and OCH₃.
Most preferably R⁶ is selected from t-butyl, 4-tetrahydropyranyl, 3-pyridazinyl, 4-pyrimidinyl, 2-pyridinyl and 3-pyridinyl, wherein the said heteroaryl group is optionally substituted by F, OH, ═O, OCH₃, and CN.
Preferably R⁷ is selected from pyridinyl and phenyl, wherein said pyridinyl or said phenyl is substituted by 1-3 groups independently selected from halo, CN, CF₃, OCF₃, OCH₃ and methyl. More preferably R⁷ is selected from pyridinyl and phenyl, wherein said pyridinyl or said phenyl is substituted by 1-2 groups independently selected from Cl, F, CN and OCH₃.
More preferably still R⁷ is 5-chloropyridin-2-yl or phenyl substituted by 1-2 groups independently selected from Cl, F, OCH₃ and CN.
Most preferably R⁷ is 5-chloropyridin-2-yl, 2,4-difluorophenyl or 4-methoxyphenyl.
Preferably R³ is selected from H, methyl, ethyl and propyl wherein said alkyl group is optionally substituted with OH or OCH₃.
More preferably R⁸ is selected from H, methyl and ethyl.
Most preferably R⁸ is selected from H and methyl.
Preferably R⁹ is selected from H, C₁-C₄alkyl and SO₂C₁-C₄alkyl.
More preferably R⁹ is selected from H and C₁-C₄alkyl.
Most preferably R⁹ is selected from H and methyl.

In a second aspect, particularly preferred embodiments of the invention are as follows:
Preferred R⁶ groups are selected from:

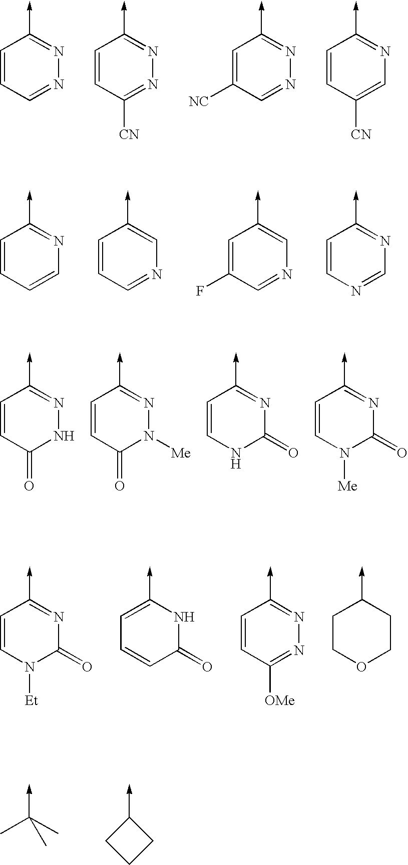

Preferred R⁷ groups are selected from:

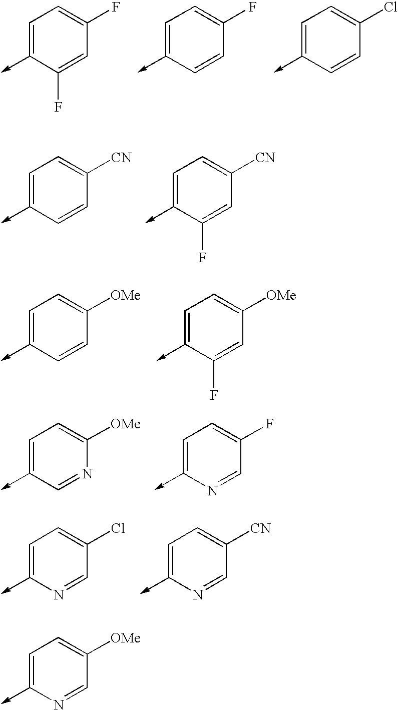

Preferred R¹⁰ groups are selected from:

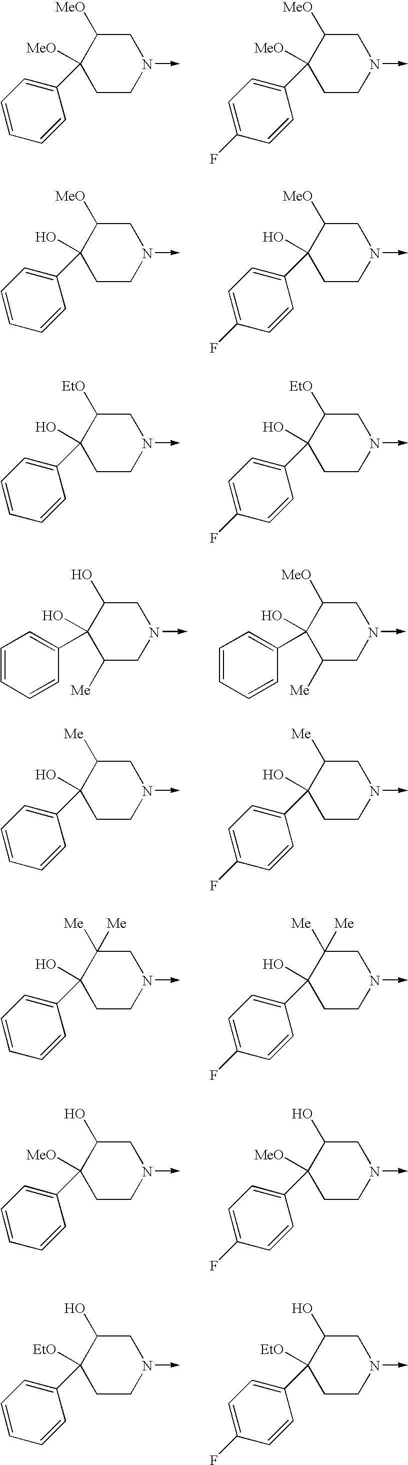

A further aspect of the invention is a compound of formula (I), and the pharmaceutically acceptable salts, hydrates, solvates, polymorphs, prodrugs, thereof, wherein each substituent is selected from the values as exemplified in the Examples below.

More particularly preferred embodiments of the present invention are the compounds of formula (I) as defined below:
(3R,4S)-1-{[(3S,4R)-4-(2,4-difluorophenyl)-1-pyridazin-3-ylpyrrolidin-3-yl]carbonyl}-3-methyl-4-phenylpiperidin-4-ol;
3-[(3R,4S)-3-(2,4-difluorophenyl)-4-{[(3S,4S)-3,4-dimethoxy-4-phenylpiperidin-1-yl]carbonyl}pyrrolidin-1-yl]pyridazine;
(3R,4R)-1-{[(3S,4R)-4-(2,4-difluorophenyl)-1-pyridazin-3-ylpyrrolidin-3-yl]carbonyl}-3-ethoxy-4-phenylpiperidin-4-ol;
3-[(3R,4S)-3-(2,4-difluorophenyl)-4-{[(3S,4S)-4-(4-fluorophenyl)-3,4-dimethoxypiperidin-1-yl]carbonyl}pyrrolidin-1-yl]pyridazine;
3-[(3R,4S)-3-(2,4-difluorophenyl)-4-{[(3R,4R)-4-(4-fluorophenyl)-3,4-dimethoxypiperidin-1-yl]carbonyl}pyrrolidin-1-yl]pyridazine
(3S,4S)-1-{[(3S,4R)-4-(2,4-difluorophenyl)-1-pyridazin-3-ylpyrrolidin-3-yl]carbonyl}-4-(4-fluorophenyl)-3-methoxypiperidin-4-ol;
(3R,4S)-1-{[(3S,4R)-4-(2,4-difluorophenyl)-1-(5-fluoropyridin-3-yl)pyrrolidin-3-yl]carbonyl}-3-methyl-4-phenylpiperidin-4-ol;
6-[(3R,4S)-3-(2,4-difluorophenyl)-4-{[(3S,4S)-3,4-dimethoxy-4-phenylpiperidin-1-yl]carbonyl}pyrrolidin-1-yl]nicotinonitrile;
(3S,4S)-1-{[(3S,4R)-4-(2,4-difluorophenyl)-1-pyridazin-3-ylpyrrolidin-3-yl]carbonyl}-3-methoxy-4-phenylpiperidin-4-ol;
(3S,4S)-1-{[(3S,4R)-4-(2,4-difluorophenyl)-1-pyridazin-3-ylpyrrolidin-3-yl]carbonyl}-4-ethoxy-4-phenylpiperidin-3-ol;
(4R)-1-{[(3S,4R)-4-(2,4-difluorophenyl)-1-pyridazin-3-ylpyrrolidin-3-yl]carbonyl}-3,3-dimethyl-4-phenylpiperidin-4-ol;
6-[(3R,4S)-3-(2,4-difluorophenyl)-4-{[(3R,4S)-4-hydroxy-3-methyl-4-phenylpiperidin-1-yl]carbonyl}pyrrolidin-1-yl]nicotinonitrile;
6-[(3R,4S)-3-(2,4-difluorophenyl)-4-{[(3S,4S)-4-hydroxy-3-methoxy-4-phenylpiperidin-1-yl]carbonyl}pyrrolidin-1-yl]nicotinonitrile;
6-[(3R,4S)-3-(2,4-difluorophenyl)-4-{[(4S)-4-hydroxy-3,3-dimethyl-4-phenylpiperidin-1-yl]carbonyl}pyrrolidin-1-yl]nicotinonitrile;
6-[(3R,4S)-3-(2,4-difluorophenyl)-4-{[(3S,4S)-3,4-dimethoxy-4-phenylpiperidin-1-yl]carbonyl}pyrrolidin-1-yl]-2-methylpyridazin-3(2H)-one;
3-[(3R,4S)-3-(2,4-difluorophenyl)-4-{[(3S,4S)-3,4-dimethoxy-4-phenylpiperidin-1-yl]carbonyl}pyrrolidin-1-yl]-6-methoxypyridazine;
6-[(3R,4S)-3-(2,4-difluorophenyl)-4-{[(3S,4S)-3,4-dimethoxy-4-phenylpiperidin-1-yl]carbonyl}pyrrolidin-1-yl]pyridazine-3-carbonitrile;
6-[(3R,4S)-3-(2,4-difluorophenyl)-4-{[(3R,4S)-4-hydroxy-3-methyl-4-phenylpiperidin-1-yl]carbonyl}pyrrolidin-1-yl]pyridazine-3-carbonitrile;
6-[(3R,4S)-3-(2,4-difluorophenyl)-4-{[(4S)-4-hydroxy-3,3-dimethyl-4-phenylpiperidin-1-yl]carbonyl}pyrrolidin-1-yl]pyridazine-3-carbonitrile;
3-[(3R,4S)-3-(4-chlorophenyl)-4-{[(3S,4S)-3,4-dimethoxy-4-phenylpiperidin-1-yl]carbonyl}pyrrolidin-1-yl]pyridazine;
3-[(3S,4R)-3-{[(3S,4S)-3,4-dimethoxy-4-phenylpiperidin-1-yl]carbonyl}-4-(4-methoxyphenyl)pyrrolidin-1-yl]pyridazine;
6-[(3S,4R)-3-{[(3S,4S)-3,4-dimethoxy-4-phenylpiperidin-1-yl]carbonyl}-4-(4-methoxyphenyl)pyrrolidin-1-yl]pyridazine-3-carbonitrile;
6-[(3S,4R)-3-{[(3S,4S)-3,4-dimethoxy-4-phenylpiperidin-1-yl]carbonyl}-4-(4-methoxyphenyl)pyrrolidin-1-yl]nicotinonitrile;
6-[(3S,4R)-3-{[(3S,4S)-3,4-dimethoxy-4-phenylpiperidin-1-yl]carbonyl}-4-(4-methoxyphenyl)pyrrolidin-1-yl]pyridazin-3(2H)-one;
6-[(3S,4S)-3-(5-chloropyridin-2-yl)-4-{[(3S,4S)-3,4-dimethoxy-4-phenylpiperidin-1-yl]carbonyl}pyrrolidin-1-yl]pyridazin-3(2H)-one;
6-[(3S,4S)-3-(5-chloropyridin-2-yl)-4-{[(3S,4S)-3,4-dimethoxy-4-phenylpiperidin-1-yl]carbonyl}pyrrolidin-1-yl]-2-methylpyridazin-3(2H)-one; and
3-[(3R,4R)-3-(2,4-difluorophenyl)-4-{[(3S,4S)-3,4-dimethoxy-4-phenylpiperidin-1-yl]carbonyl}piperidin-1-yl]pyridazine; and their pharmaceutically acceptable salts, hydrates, solvates, polymorphs and prodrugs.

As previously stated, the present invention additionally provides compounds of general formulae (IA) to (IH) as well as mixtures thereof as detailed hereinafter. For the avoidance of doubt, all references to compounds of general formula (I), including the preferred embodiments described above, hereinafter, such as for example, salts, hydrates, solvates, polymorphs, pro-drugs or optical, geometric and tautomeric isomers thereof are intended to encompass compounds having general formulae (IA) to (1H) unless otherwise specifically provided.

The pharmaceutically acceptable salts of the compounds of the formula (I) include the acid addition salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Hemisalts of the acids may also be formed, for example, hemisulphate.

For a review on suitable salts, see Handbook of Pharmaceutical Salts: Properties, Selection, and Use by Stahl and Wermuth (Wiley-VCH, 2002).

Pharmaceutically acceptable salts of compounds of formula I may be prepared by one or more of three methods:
(i) by reacting the compound of formula I with the desired acid;
(ii) by removing an acid- or base-labile protecting group from a suitable precursor of the compound of formula I or by ring-opening a suitable cyclic precursor, for example, a lactone or lactam, using the desired acid; or
(iii) by converting one salt of the compound of formula I to another by reaction with an appropriate acid or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by 0. Almarsson and M. J. Zaworotko (2004). For a general review of multi-component complexes, see J Pharm Sci, 64 (8), 1269-1288, by Haleblian (August 1975).

The compounds of the invention may also exist in a mesomorphic state (mesophase or liquid crystal) when subjected to suitable conditions. The mesomorphic state is intermediate between the true crystalline state and the true liquid state (either melt or solution), Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

Hereinafter all references to compounds of formula (I) include references to salts, solvates, multi-component complexes and liquid crystals thereof and to solvates, multi-component complexes and liquid crystals of salts thereof.

The compounds of the invention include compounds of formula (I) as hereinbefore defined, polymorphs and crystal habits thereof, prodrugs, and isomers thereof (including optical, geometric and tautomeric isomers) as hereinafter defined and isotopically labelled compounds of formula (I).

As indicated, so-called ‘prodrugs’ of the compounds of formula (I) are also within the scope of the invention. Thus certain derivatives of compounds of formula (I), which may have little or no pharmacological activity themselves, can, when administered into or onto the body, be converted into compounds of formula (I) having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T Higuchi and W Stella) and Bioreversible Carriers in Drug Design, Pergamon Press, 1987 (Ed. E B Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in Design of Prodrugs by H Bundgaard (Elsevier, 1985).

Some examples of prodrugs in accordance with the invention include
(i) where the compound of formula I contains an alcohol functionality (—OH), an ether thereof, for example, a compound wherein the hydrogen of the alcohol functionality of the compound of formula I is replaced by —(C₁-C₆)alkanoyloxymethyl, such as for example when R²═OH, or when the R³ group is substituted by an —OH group, a preferred pro-drug herein is an ether; and
(ii) where the compound of formula I contains a primary or secondary amino functionality (—NH₂ or —NHR where R≠H), such as for example where R³=H, a preferred pro-drug thereof is an amide thereof, for example, a compound wherein, as the case may be, one or both hydrogens of the amino functionality of the compound of formula I is/are replaced by —(C₁-C₁₀)alkanoyl, preferably —(C₁-C₆)alkanoyl, more preferably methyl, ethyl or propylalkanoyl.

Particularly preferred pro-drugs herein are ethers and —(C₁-C₄)alkyl ethers of the compounds of general formula (I).

Certain compounds of formula (I) may also themselves act as prodrugs of other compounds of formula (I).

Also included within the scope of the invention are metabolites of compounds of formula I, that is, compounds formed in vivo upon administration of the drug. Some examples of metabolites in accordance with the invention include
(i) where the compound of formula I contains a methyl group, an hydroxymethyl derivative thereof (—CH₃->-CH₂OH):
(ii) where the compound of formula I contains an alkoxy group, an hydroxy derivative thereof (—OR->-OH);
(iii) where the compound of formula I contains a tertiary amino group, a secondary amino derivative thereof (—NR⁷R⁸—>—NHR⁷ or —NHR⁸ where R⁸ and R⁸ are different groups);
(iv) where the compound of formula I contains a secondary amino group, a primary derivative thereof (—NHR⁷—>—NH₂);
(v) where the compound of formula I contains a phenyl moiety, a phenol derivative thereof (-Ph->-PhOH); and
   Compounds of formula (I) containing one or more asymmetric carbon atoms can exist as two or more stereoisomers. Where structural isomers are interconvertible via a low energy barrier, tautomeric isomerism (‘tautomerism’) can occur. This can take the form of proton tautomerism in compounds of formula (I) containing, for example, an imino, keto, or oxime group, or so-called valence tautomerism in compounds that contain an aromatic moiety. It follows that a single compound may exhibit more than one type of isomerism.

Included within the scope of the present invention are all stereoisomers, geometric isomers and tautomeric forms of the compounds of formula (I), including compounds exhibiting more than one type of isomerism, and mixtures of one or more thereof. Also included are acid addition salts wherein the counterion is optically active, for example, d-lactate or racemic, for example, dl-tartrate.

Specifically included within the scope of the present invention are stereoisomeric mixtures of compounds having formula (I), or a diastereomerically enriched or diastereomerically pure isomer of a compound of formula (I), or an enantiomerically enriched or enantiomerically pure isomer of a compound of formula (I).

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2 to 20%, and may contain from 0 to 5% by volume of an alkylamine. Concentration of the eluate affords the enriched mixture. The absolute composition of the mobile phase will be dependant upon the chiral stationary phase (asymmetric resin) selected.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

The present invention includes all pharmaceutically acceptable isotopically-labelled compounds of formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶CI, fluorine, such as ¹⁸F, iodine, such as ¹²³I and ¹²⁵I, nitrogen, such as ¹³N and ¹⁵N, oxygen, such as ¹⁵O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, Le. ³H, and carbon-14, i.e. ¹⁴C, are particularly useful for this purpose in view of their ease of incorporation and ready means of detection.

Substitution with heavier isotopes such as deuterium, i.e. H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds of formula (I) can generally be prepared by conventional techniques known to those skilled in the art or by processes analogous to those described in the accompanying Examples and Preparations using an appropriate isotopically-labelled reagent in place of the non-labelled reagent previously employed.

Pharmaceutically acceptable solvates in accordance with the invention include those wherein the solvent of crystallization may be isotopically substituted, e.g. D₂O, d₆-acetone, d₆-DMSO.

The routes below, including those mentioned in the Examples and Preparations, illustrate methods of synthesising compounds of formula (I). The skilled person will appreciate that the compounds of the invention could be made by methods other than those specifically described herein, by adaptation of the methods described herein and/or adaptation thereof, for example by methods known in the art. Suitable guides to synthesis, functional group interconversions, use of protecting groups, etc., are for example: “Comprehensive Organic Transformations” by R C Larock, VCH Publishers Inc. (1989); Advanced Organic Chemistry by J. March, Wiley Interscience (1985); “Designing Organic Synthesis” by S Warren, Wiley Interscience (1978); “Organic Synthesis—The Disconnection Approach” by S Warren, Wiley Inetrscience (1982); “Guidebook to Organic Synthesis” by R K Mackie and D M Smith, Longman (1982); “Protective Groups in Organic Synthesis” by T W Greene and P G M Wuts, John Wiley and Sons, Inc. (1999); and “Protecting Groups” by P J, Kocienski, George Thieme Verlag (1994); and any updated versions of said standard works.

In the general synthetic methods below, unless otherwise specified, the substituents are as defined above with reference to the compounds of formula (I) above.

Scheme 1 illustrates the preparation of compounds of formula (I) via peptide coupling of intermediates (II) and (III), if necessary adding a suitable base and/or additive (such as 1-hydroxybenzotriazole hydrate or 4-dimethylaminopyridine).

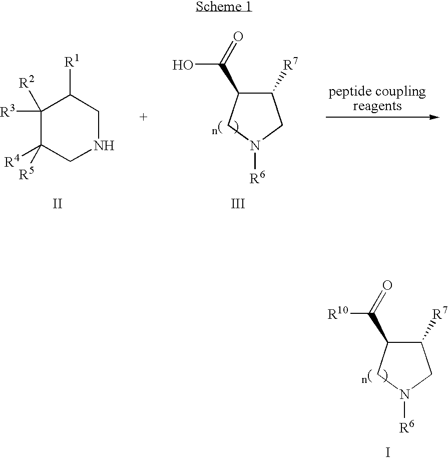

Alternative conditions employed involve stirring a solution of the piperidine (amine) of general formula (II) and the pyrrolidine (acid) of general formula (III) together with 1-(3-dimethylaminopropyl)-3-ethyl-carbodiimide hydrochloride (EDCI), triethylamine or N-methylmorpholine and 1-hydroxybenzotriazole hydrate (HOBt) in dimethylformamide (DMF), tetrahydrofuran (THF), dichloromethane (DCM) or ethyl acetate at room temperature. An alternative suitable procedure is to stir a solution of the intermediate compounds of general formula (II) and general formula (III) together with O-benzotriazol-1-yl-N,N,N′N′-tetramethyluronium hexafluorophosphate (HBTU) or 1-propylphosphonic acid cyclic anhydride in CH₂Cl₂ or EtOAc. Any suitable inert solvent may be used in place of those mentioned above, wherein inert solvent means a solvent which does not contain a carboxylic acid or primary or secondary amine. At least one equivalent of each of the coupling reagents should be used and an excess of either one or both may be used if desired.

According to a yet further embodiment the present invention provides novel intermediate compounds of general formula (II) and (III).

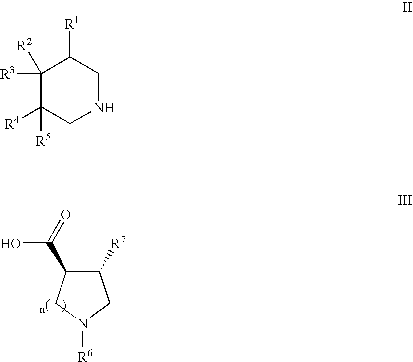

Scheme 2 illustrates an alternative route for the preparation of compounds of general formula (I), having a range of R⁶ groups, via utility of a protecting group strategy.

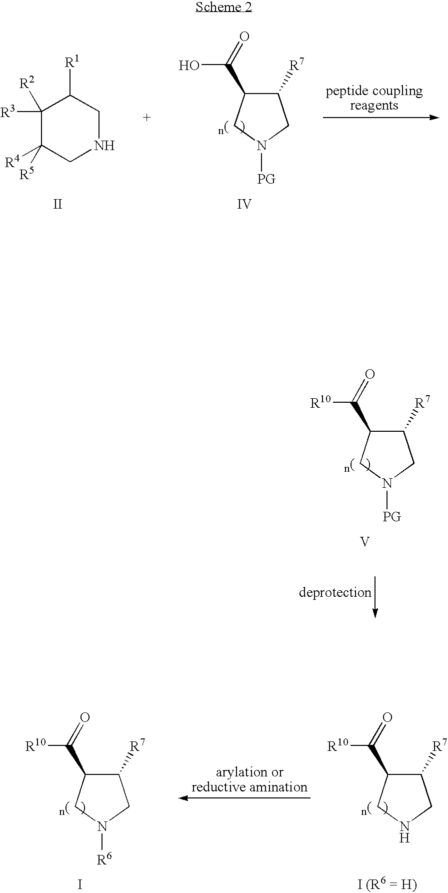

PG is a nitrogen-protecting group.

In scheme 2 the amine intermediates of general formula (II) and protected pyrrolidine acid intermediates of general formula (IV) are coupled using standard peptide coupling methods as previously described in scheme I to provide a coupled and protected intermediate of general formula (V) from which the nitrogen protecting group can be removed using standard de-protection strategies to furnish a compound of general formula (I) in which R⁶═H. Any suitable nitrogen protecting groups may be used (as described in “Protecting Groups in Organic Synthesis” 3^{rd} Edition T. W. Greene and P. G. Wuts, Wiley-Interscience, 1999). A common nitrogen protecting group (PG) suitable for use herein is tert-butoxycarbonyl, which is readily removed by treatment with an acid such as trifluoroacetic acid or hydrogen chloride in an organic solvent such as dichloromethane or 1,4-dioxane.

Alternative substituents such as alkyl and cycloalkyl groups may be introduced at R⁶ by using conventional alkylation techniques. Suitable methods for alkylation of secondary amines include:
(i) reaction with an aldehyde or ketone and a hydride reducing agent such as sodium triacetoxyborohydride, optionally in the presence of acetic acid, in an inert solvent such as dichloromethane or acetonitrile;
(ii) reaction with an alkyl halide or suitably activated alcohol derivative (e.g. as a sulfonate ester) in the presence of a base (such as triethylamine) in an inert solvent;
   Aryl and heteroaryl groups may be introduced by displacement of a suitable leaving group, for example from an aromatic or heteroaromatic precursor of formula R⁶-L where L is a suitable leaving group. Suitable leaving groups include halogens. In certain cases transition metal catalysis (e.g. palladium, copper), optionally in combination with a phosphine ligand such as 1,1′-binaphthalene-2,2′-diylbisdiphenylphosphine, may be required to achieve the required coupling products.

Alternatively, compounds of general formula (I) having particular R⁶ groups may be converted into other compounds of general formula (I) having different R⁶ groups. For example:
i) Compounds of formula (Ia), where R⁶ contains a suitable leaving group L, such as methoxy or chlorine, as shown in scheme 3, can be converted into compounds of formula (Ib), as shown in scheme 3, by hydrolysis under either acidic or basic conditions. Acidic conditions are preferred, and particularly preferred is treatment of compounds of formula (Ia) with acetic acid at reflux temperature. Alternatively, a compound of formula (Ia), where L is chloro, can be reacted with an alkoxide of formula Z-O⁻, where Z is a suitable oxygen protecting group, to give an intermediate of formula (Ia), where L is OZ. Subsequent deprotection then provides the compounds of formula (Ib). For example, when Z=benzyl, it can readily be removed by hydrogenation in the presence of a suitable catalyst

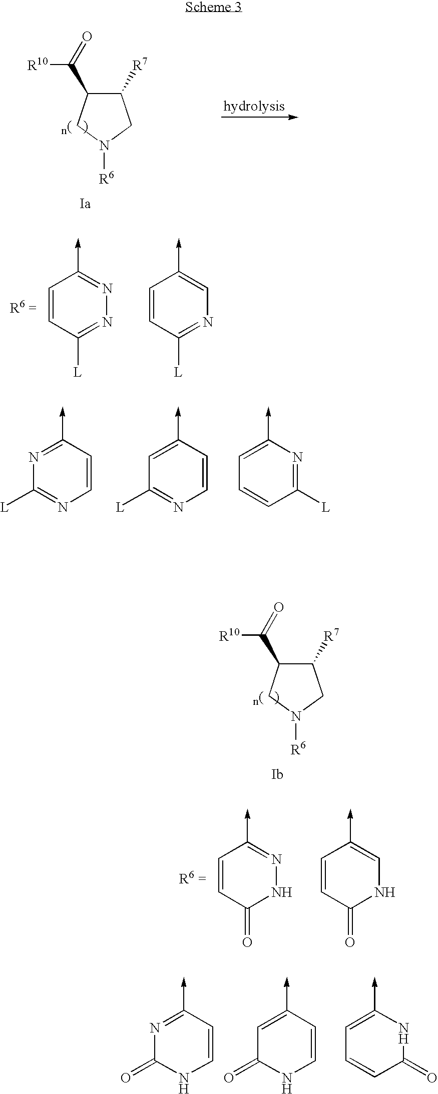

ii) Compounds of formula Ic, where R⁶ is as shown in scheme 4 and R¹¹=H, can be converted into compounds of formula Id, where R¹¹=methyl or ethyl, as shown in scheme 4, by treatment with a base and an alkylating agent in an appropriate solvent. Suitable bases include sodium hydride, lithium diisopropylamide and sodium hexamethyldisilazide, suitable alkylating agents include methyl iodide, methyl tosylate, dimethyl sulfate and ethyl iodide and suitable solvents include tetrahydrofuran, dimethylformamide and N-methyl-2-pyrrolidinone. An optional additive, such as a lithium salt, lithium bromide for example, may also be present in the reaction mixture.

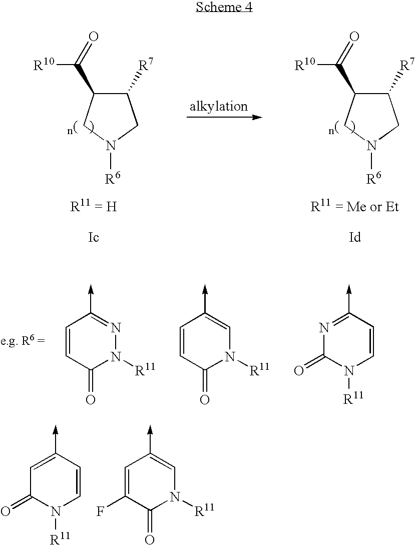

Amides and carbamate groups may be introduced at R⁶ by techniques that will be well-known to those skilled in the art by reference to literature precedents and the examples and preparations herein. Scheme 5 illustrates the route for preparation of the pyrrolidine acid intermediates of general formula (III) from unsaturated ester intermediates of general formula (VI).

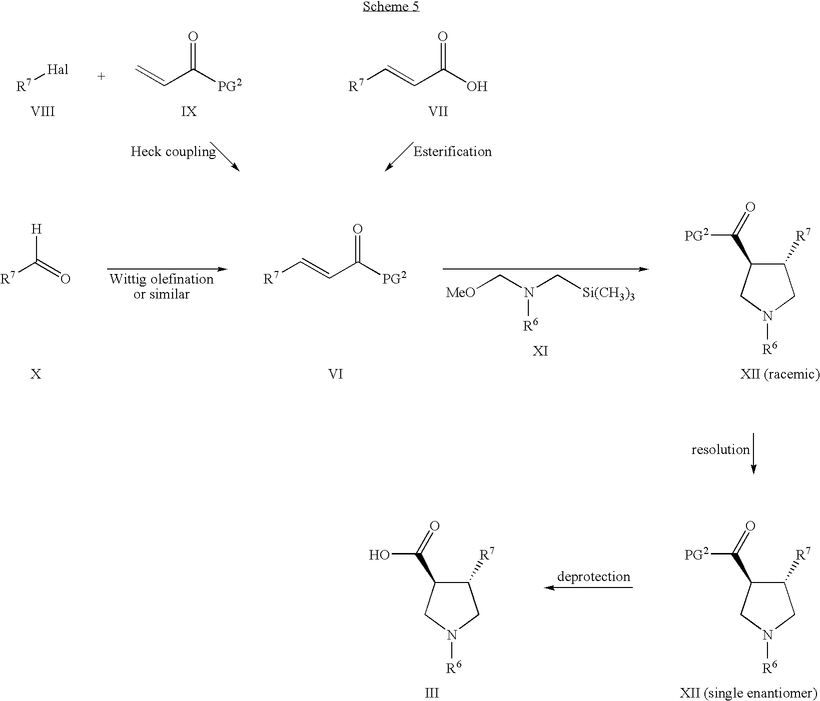

PG² is a suitable carboxylic acid protecting group. Compounds of formulae (VII), (VIII), (X) and (IX) are either commercially available or will be well-known to those skilled in the art with reference to literature precedents and/or the preparations herein.

Compounds of general formula (VI) can be made predominantly as the desired trans-isomer by Wittig or similar olefination of an aldehyde intermediate of general formula (X) with a suitable ylid e.g. methyl (triphenylphosphoranylidene)acetate, or a phosphonate anion e.g. derived from deprotonation of trimethylphosphonoacetate.

Many alternative methods exists in the literature for the production of unsaturated ester intermediates of general formula (VI), including esterification of a precursor cinnamic acid derivative (VII) using standard esterification methods, or Heck reaction of an aromatic halide (VIII) with a suitable acrylate derivative (IX), such as t-butyl acrylate, in the presence of a palladium catalyst and a suitable base, such as triethylamine.

The resulting E-olefin intermediate of general formula (VI) will undergo a [3+2]-azomethine ylid cycloaddition by reaction with an ylid precursor of general formula (XI), to provide a pyrrolidine with almost exclusively the trans-stereochemistry. This reaction requires an inert solvent such as dichloromethane or toluene or tetrahydrofuran and activation by one or more of: (1) an acid catalyst, such as TFA; (2) a desilylating agent such as silver fluoride; (3) heating.

The compound of general formula (XII) obtained from the cycloaddition reaction is a racemate and may require resolution into its constituent enantiomers, which can be achieved by preparative HPLC using a chiral stationary phase. Alternatively the acid intermediate of general formula (III) can be resolved by standard methods (e.g. formation of diastereomeric derivatives by reaction with an enantiomerically pure reagent, separation of the resulting diastereomers by physical methods and cleaving to acid (III).

Intermediate compounds of general formula (XII) can be converted into compounds of general formula (III) by deprotection of the protecting group. Many methods are available to achieve this transformation (see Advanced Organic Chemistry: Reactions, Mechanisms, and Structure, Fourth Edition. March, Jerry, 1992, pp 378-383 published by Wiley, New York, N.Y. USA). In particular, for base labile protecting groups, treatment of a compound of general formula (XII) with an aqueous alkali metal hydroxide solution, such as lithium hydroxide, sodium hydroxide or potassium hydroxide in a suitable solvent will provide the corresponding compounds of general formula (III). Preferably water-miscible organic co-solvents (such as 1,4-dioxane or tetrahydrofuran) are also utilised in such reactions. If required, the reaction may be heated to assist the hydrolysis. Deprotection of certain protecting groups may also be achieved using acid conditions e.g. by heating the protected derivative in an aqueous acid such as hydrochloric acid. Certain protecting groups are more conveniently hydrolysed in acidic conditions e.g. tert-butyl or benzhydryl esters. Such esters can be cleaved by treatment with anhydrous acids such as trifluoroacetic acid or hydrogen chloride in an inert organic solvent such as dichloromethane.

Scheme 6 illustrates an alternative route for the preparation of a single enantiomer of the pyrrolidine acid intermediate of general formula (III) from unsaturated intermediates of general formula (VI), using an oxazolidinone as a chiral auxiliary. The acid of formula (XIII) may be obtained by deprotection of (VI) and then coupled to an oxazolidinone (where R is preferably phenyl, tertiary butyl, or iso-propyl) to provide an intermediate of formula (XIV). Alternatively, the reaction of a compound of formula (VI) (when PG²=OCOt-Bu) with the lithium salt of an oxazolidinone, in a suitable solvent (e.g. THF), may also provide a compound of formula (XIII).

The compound of formula (XIV) will undergo an [3+2]-azomethine ylide cycloaddition by reaction with the compound of general formula (XI), to provide diastereomers (XV) and (XVI) which can be separated by chromatography or crystallisation and hydrolysed to give a pyrrolidine of formula (III).

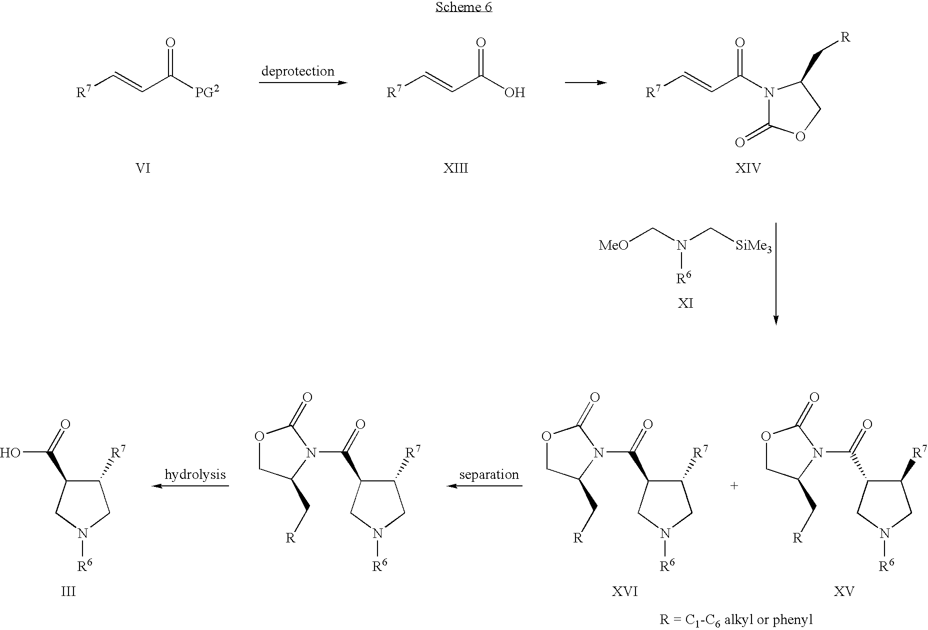

Scheme 7 illustrates that the synthesis of protected pyrrolidine acid intermediates of general formula (IV) can be achieved using a similar method to the process described hereinbefore for the intermediate of general formula (III) with the exception that the intermediate of general formula (XIIA) contains a nitrogen protecting group which may be removed subsequently in the synthetic scheme. Once the protecting group is removed, using any suitable conventional techniques, alternative R⁶ groups may be introduced by the methods described in scheme 2.

Pyrrolidines of general formula IV bearing a nitrogen protecting group may also be obtained enantioselectively by employment of an oxazolidinone chiral auxiliary, in a similar manner to that described in Scheme 6.

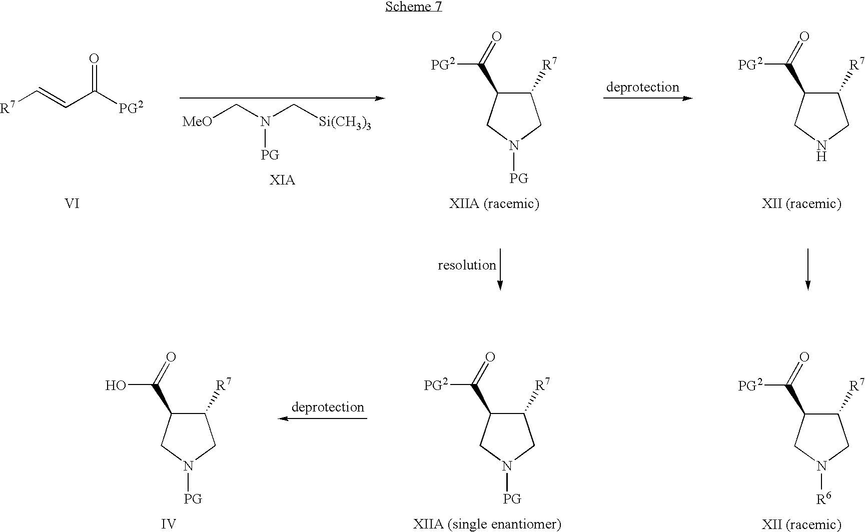

PG is selected from suitable nitrogen protecting groups. In formulae (VI), (XII) and (XIIA) PG² is selected from suitable carboxylic acid protecting groups.

Synthesis of azomethine ylid precursor compounds of general formula (XI) and (XIA) can be achieved as illustrated in scheme 8. Thus, a primary amine of general formula (XVII) may be alkylated by treatment with chloromethyltrimethylsilane, optionally neat or in an inert solvent, heating the reaction if required. The resulting intermediates (XVIII) can then be reacted with formaldehyde in methanol in the presence of a suitable base, such as potassium carbonate or tert-butylamine, to afford the intermediates (XI). To produce intermediates (XIA) containing a nitrogen protecting group a similar reaction sequence can be followed.

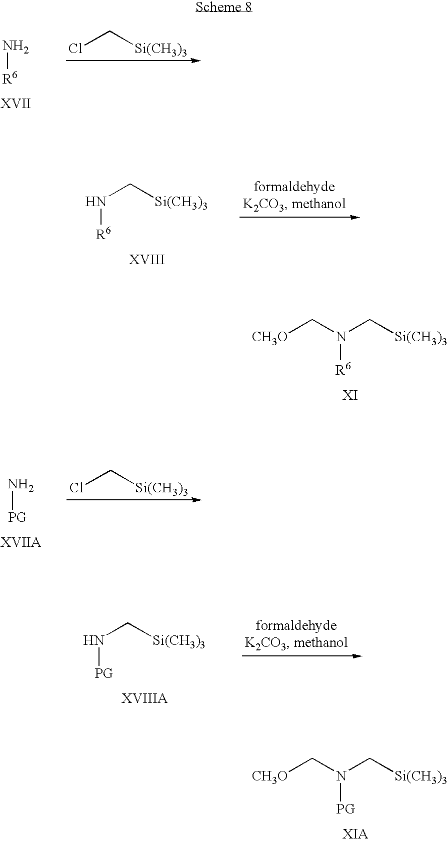

In formulae (XVIIA), (XVIIIA) and (XIA), PG is selected from suitable nitrogen protecting groups.

The piperidines of general formula (II) may be formed as mixtures of diastereomers and separation of these diastereoisomers may be achieved at an appropriate stage by conventional techniques, e.g. by fractional crystallisation, chromatography or H.P.L.C. In addition, certain of these diastereomers may be racemic and require resolution into their constituent enantiomers, which can be achieved by standard resolution techniques, such as by H.P.L.C. using a suitable chiral support or by fractional crystallisation of the diastereoisomeric salts formed by reaction of the racemate with a suitable optically active acid. Alternatively, racemic piperidines of formula (II) may be coupled to optically active acids of formula (III) or (IV) to form mixtures of diastereomers which can be separated by standard techniques e.g. by fractional crystallisation, chromatography or H.P.L.C.

As illustrated in Scheme 9, piperidine intermediates of general formula (II), where R²═OH, can be prepared by addition of organometallic nucleophiles to ketones of general formula (XIX) containing a suitable nitrogen protecting group to furnish intermediates of general formula (XX). Such nucleophilic addition is generally carried out at low temperature in an anhydrous ethereal or non-polar solvent, using Grignard, organolithium or other suitable organometallic reagent. These organometallic reagents can be made by halogen-metal exchange using a suitable halide precursor, Y—Br or Y−1 and n-butyl lithium or t-butyl lithium. Suitable protecting groups include Bn, which may be removed by hydrogenation or Boc, which may be removed by treatment with an acid such as TFA, or PMB which may be removed by treatment with DDQ, CAN or chloroethylchloroformate, to afford the desired piperidine intermediate of general formula (II). With certain protecting groups and under certain conditions the protecting group may be labile to treatment with the organometallic reagent, and so both transformations may be accomplished in one step. e.g. when PG=Boc the protecting group may sometimes be cleaved when intermediates of formula (XIX) are treated with an organometallic reagent.

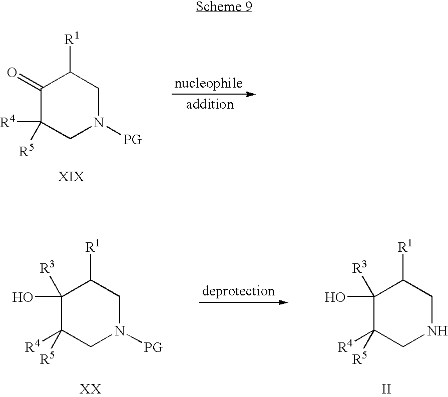

In formulae (XIX), (XX), PG is selected from suitable nitrogen protecting groups. Compounds of formula (XIX) will be well-known to those skilled in the art with reference to literature precedents and/or the preparations herein.

Scheme 10 illustrates that under forcing reduction conditions, such as hydrogenation at high pressure and or temperature, or strong acid plus triethylsilane, intermediate compounds of formula general formula (II), where R²═OH may be converted into further intermediate compounds of general formula (II) where R²═H. In certain cases protection of the piperidine nitrogen atom may be required to facilitate this transformation. Thus, intermediates of general formula (XX) may be converted into further intermediate compounds of general formula (XXI) where R═H, and then subsequently deprotected to provide compounds of general formula (II) where R²=H.

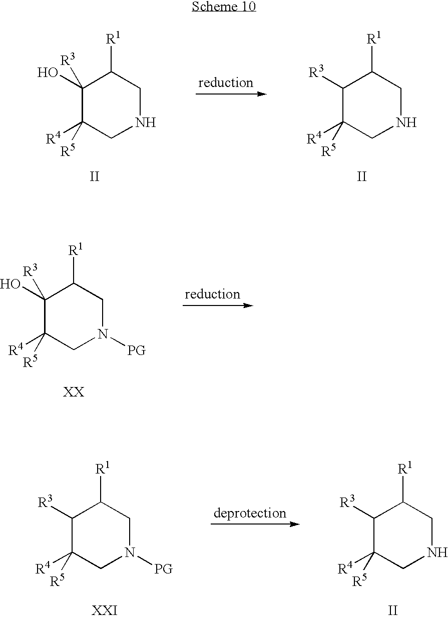

In formulae (XX) and (XXI), PG is selected from suitable nitrogen protecting groups.

As illustrated in Scheme 11, piperidine intermediates of general formula (II), where R²═NH₂, can be prepared by addition of organometallic nucleophiles to imines of general formula (XXII) containing suitable nitrogen protecting groups to furnish intermediates of general formula (XXIII). Such nucleophilic addition is generally carried out at low temperature in an anhydrous ethereal or non-polar solvent, using Grignard, organolithium or other suitable organometallic reagent. These organometallic reagents can be made by halogen-metal exchange using a suitable halide precursor, Y—Br or Y—I and n-butyl lithium or t-butyl lithium. Imines of formula (XXII) are available from ketones of formula (XIX) by reaction with the appropriate amine under suitable conditions, for example by carrying out the reaction in toluene at reflux with a Dean and Stark trap fitted to allow for azeotropic removal of water. Suitable protecting groups include Bn, which may be removed by hydrogenation, or Boc, which may be removed by treatment with an acid such as TFA, or PMB which may be removed by treatment with DDQ, CAN or chloroethylchloroformate, to afford the desired piperidine intermediate of general formula (II).

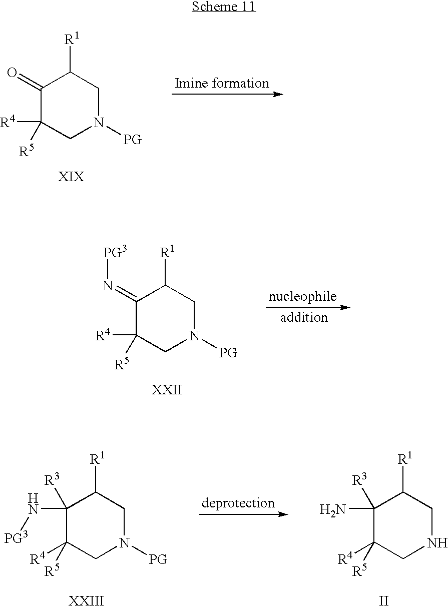

In formulae (XIX) (XXII) and (XXIII), PG and PG³ are selected from suitable nitrogen protecting groups.

As illustrated in Scheme 12, piperidine intermediates of general formula (II), where R¹═R²═OH, can be prepared by dihydroxylation of alkenes of general formula (XXIV) containing suitable nitrogen protecting groups to furnish intermediates of general formula (XXV). Many methods are available to carry out such a dihydroxylation reaction but particularly suitable is the asymmetric dihydroxylation reaction developed by Sharpless (Chemical Reviews 1994, 94, 2483) which generates a cis diol of known stereochemistry and usually in very high enantiomeric excess. Suitable protecting groups include Bn, which may be removed by hydrogenation, or Boc, which may be removed by treatment with an acid such as TFA, to afford the desired piperidine intermediate of general formula (II). Similarly, piperidine intermediates of general formula (II), where R⁴═R²═OH, can be prepared by dihydroxylation of alkenes of general formula (XXVI) to give intermediates of general formula (XXVII). Removal of the protecting group then gives the piperidine of formula (II).

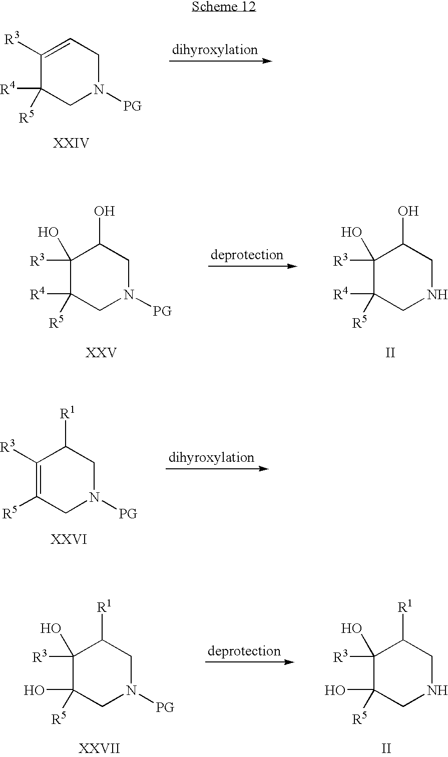

PG is selected from suitable nitrogen protecting groups. Compounds of formulae (XXIV) and (XXVI) will be well-known to those skilled in the art with reference to literature precedents and/or the preparations herein.

In addition, scheme 13 illustrates that intermediate compounds of general formula (XXV) may be converted into further intermediate compounds of general formula (XXVIII) or (XXIX) which on deprotection give piperidines of general formula (II), where R¹═OC₁-C₄alkyl, R²═OH and R¹═R²=OC₁-C₄alkyl respectively. Conversion of intermediate compounds of formula (XXV) to compounds of formula (XXIX) may be achieved by the standard Williamson ether synthesis. That is, the alcohol groups in compounds of general formula (XXV) may be deprotonated with a strong base such as sodium hydride, in an anhydrous solvent, such as tetrahydrofuran or dimethylformamide, and the resulting anion reacted with an alkyl halide, heating the reaction if necessary. Alternatively, intermediates of formula (XXV) can be converted to compounds of general formula (XXVIII) by selectively alkylating only the less hindered secondary alcohol. Suitable conditions include reacting a diol of formula (XXV) with an excess of alkyl halide in a mixture of aqueous sodium hydroxide and toluene in the presence of a phase transfer catalyst such as tetrabutylammonium hydrogen sulfate.

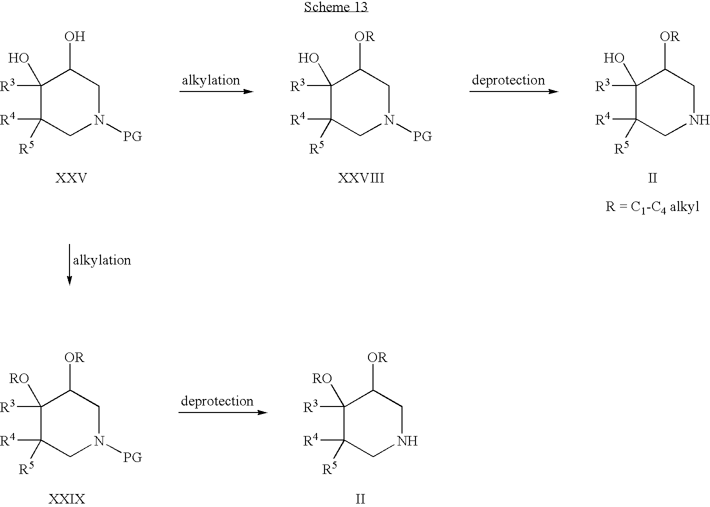

In formulae (XXV), (XXVIII) and (XXIX), PG is selected from suitable nitrogen protecting groups.

Scheme 14 illustrates the route for preparation of the piperidine acid intermediates of general formula (IV), where n=2, from dicarbonyl intermediates of general formula (XXX).

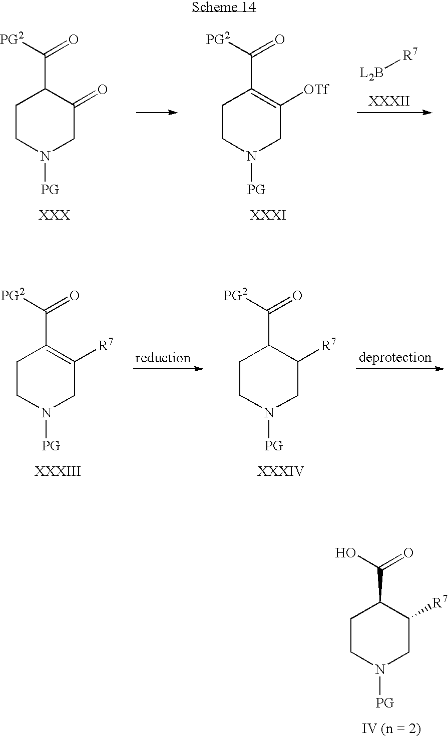

PG is selected from suitable nitrogen protecting groups and PG² is selected from suitable carboxylic acid protecting groups. L is a suitable ligand for boron, such as —OH. Compounds of formulae (XXX) and (XXXII) are either commercially available or will be well-known to those skilled in the art with reference to literature precedents and/or the preparations herein.

Compounds of general formula (XXXI) can be made by treatment of a ketone of general formula (XXX) with a suitable triflating agent, such as trifluoromethanesulfonic anhydride, in the presence of a suitable base, such as N,N-diisopropylethylamine in a suitable inert solvent, such as dichloromethane.

Compounds of general formula (XXXI) can be converted to compounds of general formula (XXXIII) by treatment with a boron derivative of formula (XXXII) under appropriate conditions in the presence of a suitable catalyst. Suitable boron derivatives include aryl or heteroaryl boronic acids, appropriate conditions include heating at 80° C. in a mixture of toluene and ethanol in the presence of sodium carbonate and appropriate catalysts include dichloro[1,1′-bis(diphenylphosphino)ferrocene]palladium.

Alkenes of general formula (XXXIII) can be converted to intermediates of general formula (XXXIV) by treatment with an appropriate reducing agent, such as magnesium in methanol or hydrogen in the presence of a suitable transition metal catalyst. Compounds of general formula (XXXIV) can be formed as mixtures of cis and trans isomers and a subsequent epimerisation step may be required to convert the undesired cis isomer to the desired trans isomer. Suitable epimerisation conditions include treatment with base in a suitable solvent, possibly at elevated temperature.

Intermediate compounds of general formula (XXXIV) can be converted into compounds of general formula (IV) by deprotection of the protecting group PG². Many methods are available to achieve this transformation (see Advanced Organic Chemistry: Reactions, Mechanisms, and Structure, Fourth Edition. March, Jerry, 1992, pp 378-383 published by Wiley, New York, N.Y. USA). In particular, base labile protecting groups, such as methyl or ethyl esters, can be deprotected by treatment with an aqueous alkali metal hydroxide solution, such as lithium hydroxide, sodium hydroxide or potassium hydroxide in a suitable solvent. Preferably water-miscible organic co-solvents (such as 1,4-dioxane or tetrahydrofuran) are also utilised in such reactions. If required, the reaction may be heated to assist the hydrolysis. Deprotection of certain protecting groups may also be achieved using acid conditions e.g. by heating the protected derivative in an aqueous acid such as hydrochloric acid. Certain protecting groups are more conveniently hydrolysed in acidic conditions e.g. tert-butyl or benzhydryl esters. Such esters can be cleaved by treatment with anhydrous acids such as trifluoroacetic acid or hydrogen chloride in an inert organic solvent such as dichloromethane.

The piperidine compound of general formula (IV), where n=2, obtained as described in scheme 14 above, is a racemate and may require resolution into its constituent enantiomers. This can be achieved by preparative HPLC using a chiral stationary phase. Alternatively the acid intermediate of general formula (IV) can be resolved by standard methods (e.g. formation of diastereomeric derivatives by reaction with an enantiomerically pure reagent, separation of the resulting diastereomers by physical methods and cleaving to acid (IV).

The skilled man will appreciate that, in addition to protecting nitrogen or acid groups, as discussed hereinbefore, at various times during the synthesis of the compounds of formula I, it may be necessary to protect further groups, such as for example, hydroxy groups with a suitable protecting group, then remove the protecting group. Methods for deprotection of any particular group will depend on the protecting group. For examples of protection/deprotection methodology see “Protective groups in Organic synthesis”, T W Greene and P G M Wutz. For example, where a hydroxy group is protected as a methyl ether, deprotection conditions could for example comprise refluxing in 48% aqueous HBr, or by stirring with borane tribromide in dichloromethane. Alternatively where a hydroxy group is protected as a benzyl ether, deprotection conditions could for example comprise hydrogenation with a palladium catalyst under a hydrogen atmosphere.

All of the above reactions and the preparations of novel starting materials used in the preceding methods are conventional and appropriate reaction conditions for their performance or preparation as well as procedures for isolating the desired products will be well-known to those skilled in the art with reference to literature precedents and the Examples and Preparations herein.

### MCR4Activity

The compounds of the present invention have utility as MCR4 agonists in the treatment of various disease states.

Preferably said MCR4 agonists exhibit a functional potency at the MC4 receptor expressed as an EC₅₀, lower than about 1000 nM, more preferably lower than 500 nM, yet more preferably lower than about 100 nM and more preferably still lower than about 50 nM wherein said EC₅₀ measurement of MCR4 functional potency can be carried out using Protocol E as described in International Patent Application publication number WO 2005/077935. Using this assay, compounds according to the present invention exhibit a functional potency at the MC4 receptor expressed as an EC₅₀ lower than 1000 nM.

The compound of example 8 has a functional potency at the MC4 receptor expressed as an EC₅₀ of 1.5 nM; the compound of example 15 has a functional potency at the MC4 receptor expressed as an EC₅₀ of 11.5 nM; and the compound of example 13 has a functional potency at the MC4 receptor expressed as an EC₅₀ of 44 nM.

Preferred compounds herein exhibit functional potency at the MCR4 receptor as defined herein before and are selective for MCR4 over MCR1. Preferably said MCR4 agonists have a selectivity for MCR4 over MCR1 wherein said MCR4 receptor agonists are at least about 10-times, preferably at least about 20-times, more preferably at least about 30-times, even more preferably at least about 100-times, more preferably still at least about 300-times, even more preferably still at least about 500-times and especially at least about 1000-times more functionally selective for a MCR4 receptor as compared with the MCR1 receptor wherein said relative selectivity assessments are based on the measurement of MCR1 and MCR4 functional potencies which can be carried out using the assays as described herein.

Preferably said MCR4 agonists have a selectivity for MCR4 over MCR3 wherein said MCR4 receptor agonists are at least about 10-times, preferably at least about 30-times, more preferably at least about 100-times, more preferably still at least about 300-times, even more preferably still at least about 500-times and especially at least about 1000-times more functionally selective for a MCR4 receptor as compared with the MCR3 receptor wherein said relative selectivity assessments are based on the measurement of MCR3 and MCR4 functional potencies which can be carried out using the assays as described herein.

Preferred compounds herein exhibit functional potency at the MCR4 receptor as defined herein before and are selective for MCR4 over MCR5. Preferably said MCR4 agonists have a selectivity for MCR4 over MCR5 wherein said MCR4 receptor agonists are at least about 10-times, preferably at least about 30-times, more preferably at least about 100-times, more preferably still at least about 300-times, even more preferably still at least about 500-times and especially about 1000-times more functionally selective for a MCR4 receptor as compared with the MCR5 receptor wherein said relative selectivity assessments are based on the measurement of MCR5 and MCR4 functional potencies which can be carried out using the assays as described herein.

Preferably said MCR4 agonists have a selectivity for MCR4 over MCR1 and MCR3 wherein said MCR4 receptors agonists are at least about 10-times, preferably at least about 30-times, more preferably at least about 100-times, more preferably still at least about 300-times, even more preferably still at least about 1000-times more functionally selective for a MCR4 receptor as compared with the MCR1 and MCR3 receptors.

Preferred compounds herein exhibit functional potency at the MCR4 receptor as defined herein before and are selective for MCR4 over MCR1 and MCR5. Preferably said MCR4 agonists have a selectivity for MCR4 over MCR1 and MCR5 wherein said MCR4 receptor agonists are at least about 10-times, preferably at least about 30-times, more preferably at least about 100-times, more preferably still at least about 300-times, even more preferably still at least about 500-times and especially at least about 1000-times more functionally selective for a MCR4 receptor as compared with the MCR1 and MCR5 receptors.

Preferably said MCR4 agonists have a selectivity for MCR4 over MCR3 and MCR5 wherein said MCR4 receptor agonists are at least about 10-times, preferably at least about 30-times, more preferably at least about 100-times, more preferably still at least about 300-times, most preferably at least about 1000-times more functionally selective for a MCR4 receptor as compared with the MCR3 and MCR5 receptors.

### Therapeutic Uses

In addition to their role in treating sexual dysfunction the compounds of the present invention are likely to be efficacious in a number of additional indications as described hereinafter. The terms “treating”, “treat”, or “treatment” as used herein are intended to embrace both prevention and control i.e., prophylactic, and palliative treatment of the indicated conditions.

The compounds of the invention likely to be useful in the treatment of diseases, disorders or conditions including, but not limited to, treating male and female sexual dysfunctions including hypoactive sexual desire disorder, sexual arousal disorder, orgasmic disorder and/or sexual pain disorder in females, male erectile dysfunction, obesity (by reducing appetite, increasing metabolic rate, reducing fat intake or reducing carbohydrate craving), diabetes mellitus (by enhancing glucose tolerance, decreasing insulin resistance), hypertension, hyperlipidemia, osteoarthritis, lower urinary tract dysfunction conditions, cancer, gall bladder disease, sleep apnea, depression, anxiety, compulsion, neuroses, insomnia/sleep disorder, substance abuse, pain, fever, inflammation, immune modulation, rheumatoid arthritis, skin tanning, acne and other skin disorders, neuroprotective and cognitive and memory enhancement including the treatment of Alzheimer's disease.

Accordingly the present invention provides for the use of a compound of formula (I) as a medicament.

Some compounds of formula (I) show highly specific activity toward the melanocortin-4 receptor making them especially useful in the treatment of male and female sexual dysfunctions, as well as obesity.

Compounds of present invention are likely to be useful in treating male and female sexual dysfunction, particularly male erectile dysfunction.

Female sexual dysfunction (FSD) includes female sexual arousal disorder (FSAD), desire disorders such as hypoactive sexual desire disorder (lack of interest in sex), and orgasmic disorders such as anorgasmia (unable to achieve orgasm).

Male sexual dysfunction includes male erectile dysfunction (MED) and ejaculatory disorders such as anorgasmia (unable to achieve orgasm) or desire disorders such as hypoactive sexual desire disorder (lack of interest in sex).

Compounds of the present invention are likely to be particularly useful in treating female sexual dysfunctions including hypoactive sexual desire disorder, sexual arousal disorder, orgasmic disorder, sexual pain disorder and male erectile dysfunction.

Compounds of the present invention are likely to be particularly suitable for treating female sexual dysfunctions, male erectile dysfunction, obesity and diabetes.

### Male Erectile Dysfunction (MED)

Male erectile dysfunction (MED), otherwise known as male erectile disorder, is defined as:
“the inability to achieve and/or maintain a penile erection for satisfactory sexual performance” (NIH Consensus Development Panel on Impotence, 1993)”

It has been estimated that the prevalence of erectile dysfunction (ED) of all degrees (minimal, moderate and complete impotence) is 52% in men 40 to 70 years old, with higher rates in those older than 70 (Melman et al 1999, J. Urology, 161, p5-11). The condition has a significant negative impact on the quality of life of the individual and their partner, often resulting in increased anxiety and tension which can lead to depression and low self-esteem. Whereas two decades ago MED was primarily considered to be a psychological disorder (Benet et al 1994 Comp. Ther., 20: 669-673), it is now known that for the majority of individuals there is an underlying organic cause. As a result, much progress has been made in identifying the mechanism of normal penile erection and the pathophysiologies of MED.

Penile erection is a haemodynamic event dependent upon the balance of contraction and relaxation of the corpus cavernosal smooth muscle and vasculature of the penis (Lerner et al 1993, J. Urology, 149, 1256-1255). Corpus cavernosal smooth muscle is also referred to herein as corporal smooth muscle or in the plural sense corpus cavernosa. Relaxation of the corpus cavernosal smooth muscle leads to an increased blood flow into the trabecular spaces of the corpus cavernosa, causing them to expand against the surrounding tunica and compress the draining veins. This produces a vast elevation in blood pressure, which results in an erection (Naylor, 1998, J. Urology, 81, 424-431).

The changes that occur during the erectile process are complex and require a high degree of coordinated control involving the peripheral and central nervous systems, and the endocrine system (Naylor, 1998, J. Urology, 81, 424-431). Corporal smooth muscle contraction is modulated by sympathetic noradrenergic innervation via activation of postsynaptic a, adrenoceptors. MED may be associated with an increase in the endogenous smooth muscle tone of the corpus cavernosum. However, the process of corporal smooth muscle relaxation is mediated partly by non-adrenergic, non-cholinergic (NANC) neurotransmission. There are a number of other NANC neurotransmitters found in the penis for example nitric oxide NO, calcitonin gene related peptide (CGRP) and vasoactive intestinal peptide (VIP). The main relaxing factor responsible for mediating this relaxation is NO, which is synthesised from L-arginine by nitric oxide synthase (NOS) (Taub et al 1993 Urology, 42, 698-704). It is thought that reducing corporal smooth muscle tone may aid NO to induce relaxation of the corpus cavernosum. During sexual arousal in the male, NO is released from neurones and the endothelium and binds to and activates soluble guanylate cyclase (sGC) located in the smooth muscle cells and endothelium, leading to an elevation in intracellular cyclic guanosine 3′,5′-monophosphate (cGMP) levels. This rise in cGMP leads to a relaxation of the corpus cavernosum due to a reduction in the intracellular calcium concentration ([Ca²⁺]ᵢ), via unknown mechanisms thought to involve protein kinase G activation (possibly due to activation of Ca²⁺ pumps and Ca²⁺-activated K⁺ channels).

Multiple potential sites have been identified within the central nervous system for the modulation of sexual behaviour. The key neurotransmitters are thought to be serotonin, norepinephrine, oxytocin, nitric oxide, dopamine and melanocortins e.g. alpha-melanocyte stimulating hormone. By mimicking the actions of one of these key neurotransmitters sexual function may be adjusted.

Melanocortins are peptides derived from pro-opiomelanocortins (POMC) that bind to and activate G-protein coupled receptors (GPCR's) of the melanocortin receptor family. Melanocortins regulate a diverse number of physiological processes including sexual function and sexual behaviour, food intake and metabolism.

There are five melanocortin receptors that have been cloned, MCR1, MCR2, MCR3, MCR4, MCR5, and are expressed in various tissues. MCR1 is specifically expressed in melanocytes and melanoma cells, MCR2 is the ACTH receptor and is expressed in adrenal tissue, MCR3 is predominately expressed in the brain and limbic system, MCR4 is widely expressed in the brain and spinal cord, and MCR5 is expressed in the brain and many peripheral tissues including skin, adipose tissue, skeletal muscle, and lymphoid tissue: MCR3 may be involved in the control of sexual function, food intake and thermogenesis. MCR4 activation has been shown to induce penile erection in rodents and MCR4 inactivation has been shown to cause obesity (reviewed in Hadley, 1999, Ann N Y Acad. Sci., 885:1-21, Wikberg et al 2000, Pharmacol Res., 42(5), 393-420).

Synthetic melanocortin receptor agonists have been found to initiate erections in men with psychogenic erectile dysfunction (Wessells et al, Int J Impot Res. 2000 October; 12 Suppl 4:S74-9.). Wessels et al describe the effects of Melanotan II (MT II), a non-selective melanocortin receptor agonist, in human subjects with erectile dysfunction (ED). MT II was administered to 20 men with psychogenic and organic ED using a double-blind placebo-controlled crossover design. Penile rigidity was monitored for 6 hours using RigiScan. Level of sexual desire and side effects were reported with a questionnaire. In the absence of sexual stimulation, Melanotan II led to penile erection in 17 of 20 men. Subjects experienced a mean of 41 minutes Rigiscan tip rigidity >80%. Increased sexual desire was reported after 13/19 (68%) doses of MT II vs. 4/21 (19%) of placebo (P<0.01). Nausea and yawning were frequently reported side effects due to MT II; at a dose of 0.025 mg/kg, 12.9% of subjects had severe nausea. Adverse reactions observed with MT-II may be the result of activation of MC-1R, MC-2R, MC-3R and/or MC 5R.

It is proposed herein that a selective MCR4 agonist can be administered orally (including buccal or sublingual administration) and will be effective in the treatment of female sexual dysfunction or male erectile dysfunction but will be devoid of significant adverse side effects such as those observed by Wessels et al i.e. a selective agent will be better tolerated.

Palatin's PT-141 is another synthetic peptide analogue of alpha-MSH. It is an agonist at melanocortin receptors including the MC3R and MC4R. Molinoff et al (Ann N.Y. Acad. Sci. (2003), 994, 96-102) describe how “administration of PT-141 to rats and nonhuman primates results in penile erections. Systemic administration of PT-141 to rats activates neurons in the hypothalamus as shown by an increase in c-Fos immunoreactivity. Neurons in the same region of the central nervous system take up pseudorabies virus injected into the corpus cavernosum of the rat penis. Administration of PT-141 (intranasally or subcutaneously) to normal men and to patients with erectile dysfunction resulted in a rapid dose-dependent increase in erectile activity.”

Use of PT-141 for sexual dysfunction is described in U.S. Pat. No. 5,576,290, U.S. Pat. No. 6,579,968 and U.S. 2002/0107,182A1. In addition, peptides such as MT-II or PT-141 are metabolised extensively in the gut and as such are most effectively administered parenterally, such as by subcutaneous, intravenous, intranasal or intramuscular route, since it is not absorbed into the systemic circulation when given by the oral route.

Thus it would be desirable to develop MCR4 agonist compounds for the treatment of male and female sexual dysfunctions suitable for oral delivery (including buccal or sublingual administration) and either reduce or overcome undesirable side effects such as nausea.

It is proposed herein that selective MCR4 agonists according to the present invention will display oral bioavailability and as such will be capable of additionally being administered orally (including buccal or sublingual administration).

There have been a number of reports illustrating that selective MCR4 agonists increase erectile activity in rats (Martin et al, 2002, Eur J. Pharmacol., 454(1), 71-79; Van Der Ploeg et al, 2002, Proc. Natl. Acad. Sci. USA., 99(17), 11381-11386). An example of a MCR4 agonist used in these studies is N-[(3R)-1,2,3,4-tetrahydroisoquinolinium-3-ylcarbonyl]-(1R)-1-(4-chlorobenzyl)-2-[4-cyclohexyl-4-(1H-1,2,4-triazol-1-ylmethyl)piperidin-1-yl]-2-oxoethylamine (1), which is a potent, selective, melanocortin subtype-4 receptor agonist (Sebhat et al, 2002, J. Med. Chem., 45(21), 4589-4593).

Cragnolini et al (Neuropeptides, 34(3-4), 211-5) have shown that alpha-MSH significantly increases lordosis sexual behaviour in female rats following injection into the ventromedial nucleus of the brain. Furthermore, they showed that HS014 (a putative MCR4 antagonist, Vergoni 1998, Eur. J. Pharmacol. 362(2-3), 95-101) dose dependently blocks the prosexual effect of alpha-MSH on lordosis in female rats. Methods of stimulating sexual response in females using various melanotropic peptides (similar to MT II) have been disclosed in U.S. Pat. No. 6,051,555.

In essence, MCR4 is an initiator of male and female sexual behaviour. Accordingly, the present invention provides for the use of a compound of formula (I) in the preparation of a medicament for the treatment of sexual dysfunction. In particular the present invention provides for the use of a compound of formula (I) in the preparation of a medicament for the treatment of male erectile dysfunction.

Patients with mild to severe MED should benefit from treatment with the compounds according to the present invention. However, early investigations suggest that the responder rate of patients with mild, moderate and severe MED may be greater with a selective MCR4 agonist/PDE5 inhibitor combination. Mild, moderate and severe MED will be terms known to the man skilled in the art, but guidance can be found in The Journal of Urology, vol. 151, 54-61 (January 1994).

Early investigations suggest the below mentioned MED patient groups should benefit from treatment with a selective MCR4 agonist and/or a PDE5i (or other combination set out hereinafter). These patient groups, which are described in more detail in Clinical Andrology vol. 23, no.4, p773-782 and chapter 3 of the book by 1. Eardley and K. Sethia “Erectile Dysfunction-Current Investigation and Management, published by Mosby-Wolfe, are as follows: psychogenic, organic, vascular, endocrinologic, neurogenic, arteriogenic, drug-induced sexual dysfunction (lactogenic) and sexual dysfunction related to cavernosal factors, particularly venogenic causes.

Accordingly the present invention provides for the use of a compound of formula (I) in the preparation of a medicament in combination with a PDE5 inhibitor for the treatment of male erectile dysfunction.

Suitable PDE5 inhibitors are described hereinafter.

### Female Sexual Dysfunction (FSD)

In accordance with the invention, FSD can be defined as the difficulty or inability of a woman to find satisfaction in sexual expression. FSD is a collective term for several diverse female sexual disorders (Leiblum, S. R. (1998)—Definition and classification of female sexual disorders. Int. J. Impotence Res., 10, S104-S106; Berman, J. R., Berman, L. & Goldstein, I. (1999)—Female sexual dysfunction: Incidence, pathophysiology, evaluations and treatment options. Urology, 54, 385-391.). The woman may have lack of desire, difficulty with arousal or orgasm, pain with intercourse or a combination of these problems. Several types of disease, medications, injuries or psychological problems can cause FSD. Treatments in development are targeted to treat specific subtypes of FSD, predominantly desire and arousal disorders.

The categories of FSD are best defined by contrasting them to the phases of normal female sexual response: desire, arousal and orgasm (Leiblum, S. R. (1998)—Definition and classification of female sexual disorders. Int. J. Impotence Res., 10, S104-S106). Desire or libido is the drive for sexual expression. Its manifestations often include sexual thoughts either when in the company of an interested partner or when exposed to other erotic stimuli. Arousal is the vascular response to sexual stimulation, an important component of which is genital engorgement and includes increased vaginal lubrication, elongation of the vagina and increased genital sensation/sensitivity. Orgasm is the release of sexual tension that has culminated during arousal.

Hence, FSD occurs when a woman has an inadequate or unsatisfactory response in any of these phases, usually desire, arousal or orgasm. FSD categories include hypoactive sexual desire disorder, sexual arousal disorder, orgasmic disorders and sexual pain disorders. Although the compounds of the invention will improve the genital response to sexual stimulation (as in female sexual arousal disorder), in doing so it may also improve the associated pain, distress and discomfort associated with intercourse and so treat other female sexual disorders.

Hypoactive sexual desire disorder is present if a woman has no or little desire to be sexual, and has no or few sexual thoughts or fantasies. This type of FSD can be caused by low testosterone levels due either to natural menopause or to surgical menopause. Other causes include illness, medications, fatigue, depression and anxiety.

Female sexual arousal disorder (FSAD) is characterised by inadequate genital response to sexual stimulation. The genitalia do not undergo the engorgement that characterises normal sexual arousal. The vaginal walls are poorly lubricated, so that intercourse is painful. Orgasms may be impeded. Arousal disorder can be caused by reduced oestrogen at menopause or after childbirth and during lactation, as well as by illnesses, with vascular components such as diabetes and atherosclerosis. Other causes result from treatment with diuretics, antihistamines, antidepressants e.g. selective serotonin reuptake inhibitors (SSRIs) or antihypertensive agents.

Sexual pain disorders (includes dyspareunia and vaginismus) is characterised by pain resulting from penetration and may be caused by medications which reduce lubrication, endometriosis, pelvic inflammatory disease, inflammatory bowel disease or urinary tract problems.

As previously discussed, MCR4 is thought to be an initiator of sexual behaviour. The clitoris is considered to be a homologue of the penis (Levin, R. J. (1991), Exp. Clin. Endocrinol., 98, 61-69); the same mechanism that provides an erectile response in the male produces an increase in genital blood flow in the female with an associated effect upon FSD. In addition there are changes in proceptivity and receptivity (lordosis).

Thus, in accordance with a preferred aspect of the invention, there is provided use of a compound of formula (I) in the preparation of a medicament for the treatment or prophylaxis of female sexual dysfunction, more particularly hypoactive sexual desire disorder, sexual arousal disorder, orgasmic disorder and sexual pain disorder.

Preferably the compounds of formula (I) are useful in the treatment or prophylaxis of sexual arousal disorder, orgasmic disorder, and hypoactive sexual desire disorder, and most preferably in the treatment or prophylaxis of sexual arousal disorder.

In a preferred embodiment the compounds of formula (I) are useful in the treatment of a subject with female sexual arousal disorder and concomitant hypoactive sexual desire disorder.

The Diagnostic and Statistical Manual (DSM) IV of the American Psychiatric Association defines Female

Sexual Arousal Disorder (FSAD) as being:

“ . . . a persistent or recurrent inability to attain or to maintain until completion of the sexual activity adequate lubrication-swelling response of sexual excitement The disturbance must cause marked distress or interpersonal difficulty.”

The arousal response consists of vasocongestion in the pelvis, vaginal lubrication and expansion and swelling of the external genitalia. The disturbance causes marked distress and/or interpersonal difficulty.

FSAD is a highly prevalent sexual disorder affecting pre-, per- and post-menopausal (+hormone replacement therapy (HRT)) women. It is associated with concomitant disorders such as depression, cardiovascular diseases, diabetes and urogenital (UG) disorders.

The primary consequences of FSAD are lack of engorgement/swelling, lack of lubrication and lack of pleasurable genital sensation. The secondary consequences of FSAD are reduced sexual desire, pain during intercourse and difficulty in achieving an orgasm.

It has recently been hypothesized that there is a vascular basis for at least a proportion of patients with symptoms of FSAD (Goldstein et al., Int. J. Impot. Res., 10, S84-S90, 1998) with animal data supporting this view (Park et al., Int. J. Impot. Res., 9, 27-37, 1997).

R. J. Levin teaches us that because “ . . . male and female genitalia develop embryologically from the common tissue anlagen, [that] male and female genital structures are argued to be homologues of one another. Thus the clitoris is the penile homologue and the labia homologues of the scrotal sac.” (Levin, R. J. (1991), Exp. Clin. Endocrinol., 98, 61-69).

Drug candidates for treating FSAD, which are under investigation for efficacy, are primarily erectile dysfunction therapies that promote circulation to male genitalia.

The compounds of the present invention are advantageous by providing a means for restoring a normal sexual arousal response—namely increased genital blood flow leading to vaginal, clitoral and labial engorgement. This will result in increased vaginal lubrication via plasma transudation, increased vaginal compliance and increased genital sensitivity. Hence, the present invention provides a means to restore, or potentiate, the normal sexual arousal response.

Thus, in accordance with a preferred aspect of the invention, there is provided use of a compound of formula (I) in the preparation of a medicament for the treatment of female sexual arousal disorder.

By female genitalia herein we mean: “The genital organs consist of an internal and external group. The internal organs are situated within the pelvis and consist of ovaries, the uterine tubes, uterus and the vagina. The external organs are superficial to the urogenital diaphragm and below the pelvic arch. They comprise the mons pubis, the labia majora and minora pudendi, the clitoris, the vestibule, the bulb of the vestibule, and the greater vestibular glands” (Gray's Anatomy, C. D. Clemente, 13^{th} American Edition).

The compounds of the invention find application in the following sub-populations of patients with FSD: the young, the elderly, pre-menopausal, peri-menopausal, post-menopausal women with or without hormone replacement therapy.

The compounds of the invention find application in patients with FSD arising from:—
i) Vasculogenic etiologies e.g. cardiovascular or atherosclerotic diseases, hypercholesterolemia, cigarette smoking, diabetes, hypertension, radiation and perineal trauma, traumatic injury to the iliohypogastric pudendal vascular system;
ii) Neurogenic etiologies such as spinal cord injuries or diseases of the central nervous system including multiple sclerosis, diabetes, Parkinsonism, cerebrovascular accidents, peripheral neuropathies, trauma or radical pelvic surgery;
iii) Hormonal/endocrine etiologies such as dysfunction of the hypothalamiclpituitary/gonadal axis, or dysfunction of the ovaries, dysfunction of the pancreas, surgical or medical castration, androgen deficiency, high circulating levels of prolactin e.g. hyperprolactinemia, natural menopause, premature ovarian failure, hyper and hypothyroidism;
iv) Psychogenic etiologies such as depression, obsessive compulsive disorder, anxiety disorder, postnatal depression/“Baby Blues”, emotional and relational issues, performance anxiety, marital discord, dysfunctional attitudes, sexual phobias, religious inhibition or a traumatic past experiences; and/or
v) Drug-induced sexual dysfunction resulting from therapy with selective serotonin reuptake inhibitors (SSR is) and other antidepressant therapies (tricyclics and major tranquilizers), anti-hypertensive therapies, sympatholytic drugs, chronic oral contraceptive pill therapy.

### Obesity

MC4-R is a G-protein-coupled seven-transmembrane receptor primarily expressed in the hypothalamus, hippocampus, and thalamus (Gantz et al. 1993 J Biol Chem 268:15174-15179). The receptor is implicated in the central regulation of body weight: MC4-R is activated by α-melanocyte-stimulating hormone (MSH), which is derived from pro-opiomelanocortin and is antagonized by agouti-related protein. α-MSH induces weight loss, whereas the ectopic expression of agouti protein results in obesity in the agouti mice (Fan et al. 1993 Nature 385:165-168; Lu et al. 1994 Nature 371:799-802). Additional evidence for the role of MC4-R in weight regulation stems from both a knockout model in mice (Huszar et al. 1997 Cell 88:131-141) and haploinsufficiency mutations in humans (Vaisse et al. 1998 Nat Genet. 20:113-114; Yeo et al. 1998 Nat Genet. 20:111-112; Hinney et al. 1999 J Clin Endocrinol Metab 84:1483-1486). In MC4-R-knockout mice, an increased body weight was discernible by age 5 wk. By age 15 wk, homozygous mutant females were, on average, twice as heavy as their wild-type littermates, whereas homozygous mutant males were ˜50% heavier than wild-type controls. Mice heterozygous for the MC4-R knockout showed a weight gain intermediate to that seen in wild-type and homozygous mutant littermates, thus demonstrating a gene dosage effect of MC4-R ablation on body-weight regulation. The food intake of homozygous mutants was increased by ˜50% in comparison to that in wild-type sibs (Huszar et al. 1997 Cell 88:131-141). [From Am. J. Hum. Genet, 65:1501-1507, 1999]. Hence it is thought that the compounds of the present invention may be useful pharmaceutical agents for the treatment of obesity as well as diseases, conditions and/or disorders related to obesity.

Accordingly the present invention provides for the use of a compound of formula (I) in the preparation of a medicament for the treatment of obesity. Furthermore, the present invention additionally provides for the use of a compound of formula (I) in the preparation of a medicament for the treatment of a disease, condition and/or disorder related to obesity.

The compounds of this invention may also be useful in conjunction with other pharmaceutical agents for the treatment of diseases, conditions and/or disorders related to obesity. Therefore, compositions (or medicaments) for use in treating obesity that include compounds of the present invention in combination with anti-obesity agents are also provided. Suitable anti-obesity agents include cannabinoid 1 (CB-1) receptor antagonists (such as rimonabant), apolipoprotein-B secretion/microsomal triglyceride transfer protein (apo-B/MTP) inhibitors (in particular, gut-selective MTP inhibitors, such as edipatapide or dirlotapide), 11β-hydroxy steroid dehydrogenase-1 (11β-HSD type 1) inhibitors, peptide YY₃₋₃₆ and analogs thereof, cholecystokinin-A (CCK-A) agonists, monoamine reuptake inhibitors (such as sibutramine), sympathomimetic agents, β₃ adrenergic receptor agonists, dopamine receptor agonists (such as bromocriptine), melanocyte-stimulating hormone receptor analogs, 5HT2c receptor agonists, melanin concentrating hormone antagonists, leptin (the OB protein), leptin analogs, leptin receptor agonists, galanin antagonists, lipase inhibitors (such as tetrahydrolipstatin, i.e. orlistat), anorectic agents (such as a bombesin agonist), Neuropeptide-Y receptor antagonists (in particular, NPY-5 receptor antagonists), thyromimetic agents, dehydroepiandrosterone or an analog thereof, glucocorticoid receptor agonists or antagonists, orexin receptor antagonists, glucagon-like peptide-1 receptor agonists, ciliary neurotrophic factors (such as Axokine™ available from Regeneron Pharmaceuticals, Inc., Tarrytown, N.Y. and Procter & Gamble Company, Cincinnati, Ohio), human agouti-related protein (AGRP) inhibitors, ghrelin receptor antagonists, histamine 3 receptor antagonists or inverse agonists, neuromedin U receptor agonists and the like. Other anti-obesity agents, including the preferred agents set forth hereinbelow, are well known, or will be readily apparent in light of the instant disclosure, to one of ordinary skill in the art. The compounds of the present invention may also be administered in combination with a naturally occurring compound that acts to lower plasma cholesterol levels. Such naturally occurring compounds are commonly called nutraceuticals and include, for example, garlic extract, Hoodia plant extracts, and niacin.

Especially preferred are anti-obesity agents selected from the group consisting of CB-1 antagonists, gut-selective MTP inhibitors, orlistat, sibutramine, bromocriptine, ephedrine, leptin, peptide YY3-36 and analogs thereof, and pseudoephedrine. Preferably, compounds of the present invention and combination therapies for the treatment of obesity and related conditions are administered in conjunction with exercise and a sensible diet.

Preferred CB-1 antagonists include Rimonabant (SR141716A also known under the tradename Acomplia™ available from Sanofi-Synthelabo) described in U.S. Pat. No. 5,624,941; and compounds described in U.S. Pat. Nos. 5,747,524, 6,432,984 and 6,518,264; U.S. Patent Publication Nos. US2004/0092520, US2004/0157839, US2004/0214855, and US2004/0214838; U.S. patent application Ser. No. 10/971,599 filed on Oct. 22, 2004; and PCT Patent Publication Nos. WO 02/076949, WO 03/075660, WO04/048317, WO04/013120, and WO 04/012671.

Preferred gut-selective MTP inhibitors include dirlotapide described in U.S. Pat. No. 6,720,351; 4-(4-(4-(4-((2-((4-methyl-4H-1,2,4-triazol-3-ylthio)methyl)-2-(4-chlorophenyl)-1,3-dioxolan-4-yl)methoxy)phenyl)piperazin-1-yl)phenyl)-2-sec-butyl-2H-1,2,4-triazol-3(4H)-one (R103757) described in U.S. Pat. Nos. 5,521,186 and 5,929,075; and implitapide (BAY 13-9952) described in U.S. Pat. No. 6,265,431.

Other representative anti-obesity agents for use in the combinations, pharmaceutical compositions, and methods of the invention can be prepared using methods known to one of ordinary skill in the art, for example; sibutramine can be prepared as described in U.S. Pat. No. 4,929,629; bromocriptine can be prepared as described in U.S. Pat. Nos. 3,752,814 and 3,752,888; orlistat can be prepared as described in U.S. Pat. Nos. 5,274,143; 5,420,305; 5,540,917; and 5,643,874; and PYY₃-36 (including analogs) can be prepared as described in US Publication No. 2002/0141985 and WO 03/027637.

### Combination Therapy

The compounds of the present invention may be delivered in combination with an auxiliary active agent for the treatment of sexual dysfunction, obesity or diabetes. Suitable auxiliary active agents for use in the combinations of the present invention include:
1) Compounds which modulate the action of naturetic factors in particular atrial naturetic factor (also known as atrial naturetic peptide), B type and C type naturetic factors such as inhibitors or neutral endopeptidase and in particular the compounds described and claimed in WO 02/02513, WO 02/03995, WO 02/079143 and EP-A-1258474, and especially the compound of Example 22 of WO 02/079143 (2S)-2{[1-{3-4(-chlorophenyl)propyl]amino}carbonyl)-cyclopentyl]methyl}-4-methoxybutanoic acid;
2) Compounds which inhibit angiotensin-converting enzyme such as enapril, and combined inhibitors of angiotensin-converting enzyme and neutral endopeptidase such as omapatrilat;
3) Substrates for NO-synthase, such as L-arginine;
4) Cholesterol lowering agents such as statins (e.g. atorvastatin/Lipitor-trade mark) and fibrates;
5) Estrogen receptor modulators and/or estrogen agonists and/or estrogen antagonists, preferably raloxifene or lasofoxifene, (−)-cis-6-phenyl-5-[4-(2-pyrrolidin-1-yl-ethoxy)-phenyl]-5,6,7,8-tetrahydronaphthalene-2-ol and pharmaceutically acceptable salts thereof the preparation of which is detailed in WO 96/21656;
6) A PDE inhibitor, more particularly a PDE 2, 3, 4, 5, 7 or 8 inhibitor, preferably PDE2 or PDE5 inhibitor and most preferably a PDE5 inhibitor (see hereinafter), said inhibitors preferably having an IC50 against the respective enzyme of less than 100 nM (with the proviso that PDE 3 and 4 inhibitors are only administered topically or by injection to the penis);
7) Vasoactive intestinal protein (VIP), VIP mimetic, VIP analogue, more particularly mediated by one or more of the VIP receptor subtypes VPAC1, VPAC or PACAP (pituitory adenylate cyclase activating peptide), one or more of a VIP receptor agonist or a VIP analogue (e.g. Ro-125-1553) or a VIP fragment, one or more of a α-adrenoceptor antagonist with VIP combination (e.g. Invicorp, Aviptadil);
8) A serotonin receptor agonist, antagonist or modulator, more particularly agonists, antagonists or modulators for 5HT1A (including VML 670 [WO02/074288] and flibanserin [US2003/0104980]), 5HT2A, 5HT2C, 5HT3 and/or SHT6 receptors, including those described in WO-09902159, WO-00002550 and/or WO-00028993;
9) A testosterone replacement agent (including dehydroandrostendione), testosterone (e.g. Tostrelle, LibiGel), dihydrotestosterone or a testosterone implant;
10) Selective androgen receptor modulators e.g. LGD-2226;
11) Estrogen, estrogen and medroxyprogesterone or medroxyprogesterone acetate (MPA) (i.e. as a combination), or estrogen and methyl testosterone hormone replacement therapy agent (e.g. HRT especially Premarin, Cenestin, Oestrofeminal, Equin, Estrace, Estrofem, Elleste Solo, Estring, Eastraderm TTS, Eastraderm Matrix, Dermestril, Premphase, Preempro, Prempak, Premique, Estratest, Estratest HS, Tibolone);
12) A modulator of transporters for noradrenaline, dopamine and/or serotonin, such as bupropion, GW-320659;
13) An agonist or modulator for oxytocin/vasopressin receptors, preferably a selective oxytocin agonist or modulator; and
14) An agonist or modulator for dopamine receptors, preferably a D3 or D4 selective agonist or modulator e.g. apomorphine.

Preferred herein are combinations of the compounds of the present invention and one or more additional therapeutic agents selected from: PDE5 inhibitors; NEP inhibitors; D3 or D4 selective agonists or modulators; estrogen receptor modulators and/or estrogen agonists and/or estrogen antagonists; testosterone replacement agents, testosterone or a testosterone implant; estrogen, estrogen and medroxyprogesterone or medroxyprogesterone acetate (MPA), or estrogen and methyl testosterone hormone replacement therapy agent.

Preferred combinations for the treatment of MED are combinations of the compounds of the present invention and one or more PDE5 inhibitors and/or NEP inhibitors.

Preferred combinations for the treatment of FSD are combinations of the compounds of the present invention and PDE5 inhibitors, and/or NEP inhibitors, and/or D3 or Dr4 selective agonists or modulators, and/or estrogen receptor modulators, estrogen agonists, estrogen antagonists, and/or testosterone replacement agents, testosterone, testosterone implant, and/or estrogen, estrogen and medroxyprogesterone or medroxyprogesterone acetate (MPA), estrogen and methyl testosterone hormone replacement therapy agent.

Particularly preferred PDE5 inhibitors for such combined products for the treatment of MED or FSD are 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil, particularly sildenafil citrate); (6R,12aR)-2,3,6,7,12,12α-hexahydro-2-methyl-6-(3,4-methylenedioxyphenyl)-pyrazino[2′, 1′:6, 1]pyrido[3,4-b]indole-1,4-dione (IC-351 or tadalafil); 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil); 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one; 5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one; 5-[2-ethoxy-5-(4-ethyl-piperazin-1-ylsulphonyl)pyridin-3-yl]-3-ethyl-2-[2-methoxyethyl]-2,6-dihydro-7H-pyrazolo[4,3-d]pyrimidin-7-one; 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide (TA-1790); 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide (DA 8159) and pharmaceutically acceptable salts thereof.

Particularly preferred NEP inhibitors for such combined products for the treatment of MED or FSD are the compounds exemplified in WO 02/079143.

Non selective melanocortin agonists are known to cause nausea and emesis and so it could be advantageous to administer compounds of the present invention alongside a suitable anti-emetic agent, for example a 5-HT₃ antagonist or a neurokinin-1 (NK-1) antagonist.

Suitable 5-HT₃ antagonists include, but are not limited to, granisetron, ondensetron, tropisetron, ramosetron, palonosetron, indisetron, dolasetron, alosetron and assertion.

Suitable NK-1 antagonists include, but are not limited to, aprepitant, casopitant, ezlopitant, cilapitant, netupitant, vestipitant, vofopitant and 2-(R)-(1-(R)-3,5-bis(trifluoromethyl)phenyl)ethoxy-4-(5-(dimethylamino)methyl-1,2,3-triazol-4-yl)methyl-3-(S)-(4-fluorophenyl)morpholine. See for example International Patent Application publication number WO2006/049933.

By cross reference herein to compounds contained in patents and patent applications which can be used in accordance with invention, we mean the therapeutically active compounds as defined in the claims (in particular of claim 1) and the specific examples (all of which is incorporated herein by reference).

If a combination of active agents is administered, then they may be administered simultaneously, separately or sequentially.

### Biological Assays

Melanocortin receptor agonist activity; selectivity
Measurement of in vitro agonist potency (EC₅₀) of compounds against melanocortin receptors type 1 and 3 (MC1 and MC3).

Activation of melanocortin (MC) receptors by agonists results in activation of intracellular adenylate cyclase enzymes that synthesise the second messenger signalling molecule, adenosine 3′,5′-cyclic monophosphate (cAMP). Changes in cAMP levels following treatment of recombinant MC1 and MC3 cell lines with test compound were measured and an MC1 and MC3 potency estimate (EC₅₀) calculated as follows:

Human embryonic kidney (HEK) or Chinese hamster ovary cell lines stably transfected with full length cDNA encoding human MC1 or MC3 receptors, respectively, were established using standard molecular biology methods. Test compounds were dissolved in dimethyl sulfoxide (DMSO) at 4 mM. 11 point half log unit increment dilution series of test compound, typically starting at 50 uM were prepared in a buffer comprised of phosphate buffered saline (PBS), 2.5% DMSO and 0.05% pluronic F-127 surfactant. Freshly cultured cells at 80-90% confluence were harvested and re-suspended in Dulbecco's Modified Eagle's Medium (DMEM). Cells (10,000 for MC3, 20,000 for MC1) were added to the test compound dilution series in a 384 well assay plate and incubated for 1 hour at 37° C. The relative cAMP concentration in each well was then measured using a β-galactosidase enzyme fragment complementation method purchased in kit form as the Discoverx cAMP II kit from GE Healthcare/Amersham Biosciences UK. In the case of MC1, 3-Isobutyl-1-methylxanthine (IBMX) at a concentration of 750 μM was included in DMEM as the cells were re-suspended for assay. The fluorescence readings taken from each assay well were converted into percent effect relative to maximum control wells corresponding to a concentration of alpha melanocyte stimulating hormone demonstrated to give a maximal effect. Sigmoidal curves were fitted to plots of log₁₀ inhibitor concentration vs percent effect using a custom made software application called SIGHTS and EC₅₀ estimates determined by the software as the concentration of test compound giving an effect half way between the bottom and top assymptopes of the sigmoidal dose response curve. Each experiment included an EC₅₀ determination for alpha melanocyte stimulating hormone, which was used as a standard to track assay consistency and allow fair comparison between EC₅₀ estimates obtained in different experiments.

MC5 and MC4 activity was determined as described by assay protocols D and E, respectively, in US2005/0176772 (pages 28-30).

### AGRP Inhibition

Agouti related protein (AGRP) is a high affinity endogenous antagonist for the MC4 receptor (Lu et al., 1994, Nature 371: 799-802; Oilman et al., 1997, Science 278: 135-138). AGRP levels are upregulated by fasting (Mizuno & Mobbs 1999, Endocrinology. 140: 4551-4557) and therefore it is important to assess the ability of anti-obesity agents acting through the MC4 receptor to inhibit the binding of AGRP. It has been ascertained that this C-terminal fragment of AGRP contains the MC4R binding determinants (Yang et al., 1999, Mol Endocrinol 13: 148-155), therefore, compounds can be evaluated for their ability to inhibit AGRP binding to membranes from cells expressing the MC4R using a competition binding assay versus [¹²⁵I]AGRP(87-132). To this end cells expressing the MC4R were subject to homogenisation and the membrane fragment isolated by differential centrifugation. CHO—CRE MC4R cell membranes (12 μg protein) were incubated with 0.3 nM [¹²⁵I]AGRP(87-132) and 11 half-log concentrations of competitor ligand, in duplicate, in a total volume of 100 μl buffer (25 mM HEPES, 1 mM MgCl₂, 2.5 mM CaCl₂, 0.5% BSA pH 7.0). Non-specific binding was determined by the inclusion of 1 μM SHU9119. The reaction was initiated by the addition of membranes and plates were incubated at room temperature for 2 hours. The reaction was terminated by rapid filtration onto GF/C filters (presoaked in 1% PEI) using a vacuum harvester followed by five 200 μl washes of ice cold wash buffer (Binding buffer containing 500 mM NaCl). The filters were soaked in 5011 scintillation fluid and the amount of radioactivity present was determined by liquid scintillation counting. Ki values were determined by data analysis using appropriate software.

Preferably the compounds of the present invention exhibit a binding constant at the MC4 receptor expressed as an Ki value against AGRP of lower than about 100 nM, more preferably lower than 500 nM, yet more preferably lower than about 100 nM and more preferably still lower than about 50 nM, wherein said Ki value is determined using the assay described above. Using this assay, compounds according to the present invention exhibit a binding constant at the MC4 receptor expressed as an Ki value against AGRP lower than 1000 nM.

Food Intake Study: To assess the efficacy of an MC4 agonist on food intake and body weight over a 24 hour period in the male rat

Rats will be acclimatized to single housing and reverse lighting conditions (9.30 am-9.30 pm) for approximately two weeks before the start of the study. Rats will be acclimatized to the Technical Scientific Equipment (TSE) cages approximately 24 hours prior to the study day. Rats will be randomly assigned to a treatment group on the morning of the study based on its weight (n=5/treatment). Each rat will either receive the MC4 agonist or vehicle orally just before lights go out. Following dosing the rat will be immediately placed back in to the TSE cage and food intake and water consumption will be monitored throughout the course of the study (24 hours). Locomotor activity will also be monitored in the form of light beam breaks.

At the end of the study rats will be killed by exsanguination under terminal anaesthesia by Isoflurane. Blood will be removed from the rat by cardiac puncture and analysed for drug concentration levels and biomarkers.

The data are expressed as mean ±SEM and comparisons between the control and the treatment is analysed by ANOVA. Statistical significance is accepted at a level of p<0.05.

### In Vitro Metabolism Rate Determination (HLM Assay)

Many drugs are metabolised by the cytochrome P450 mono-oxygenase system. This enzyme is found in high concentrations in the liver and is bound to the endoplasmic reticulum of the hepatocyte. The enzyme system can be obtained in semi-purified state by the preparation of the hepatic microsomal fractions. Determining a compound's in vitro half-life in such a system provides a useful indicator of metabolic stability.

### Materials And Reagents

All reagents are ANALAR grade.

1. 200 mM Phosphate buffer (Sigma)-100 ml 1M Phosphate buffer pH7.4 dissolved with 400 ml MilliQ water. If necessary, pH should be adjusted with concentrated orthophosphoric acid to pH 7.4, made up monthly and stored refrigerated (2-8° C.).

2. 0.1M MgCl₂.6H₂O (BDH)-2.032 g dissolved in 100 ml MilliQ water, and stored refrigerated (2-8° C.).

3. 0.02M NADP (Sigma)-15.3 mg dissolved in 1000 μl MilliQ water—and then stored refrigerated (2-8° C.) for further use.

4. 0.1M D-L Isocitric acid (Sigma)-129 mg dissolved in 5 ml MilliQ water—and then stored refrigerated (2-8° C.) for further use.

5. Isocitric dehydrogenase, Type IV (Sigma)-stored refrigerated (2-8° C.).

6. Stock solution of substrate (approximately 1 mg/ml) in miscible organic solvents such as methanol, ethanol or water, stored refrigerated (2-8° C.).

7. 50 mM p-Nitroanisole (PNA) (Aldrich)-7.65 mg dissolved in 1 ml methanol, and stored refrigerated (2-8° C.) until ready for use.

8. 50 μM p-Nitrophenol (PNP) (Sigma)-0.69 mg dissolved in 100 ml water and stored refrigerated (2-8° C.).

9. 20% Trichloroacetic acid (TCA) (BDH)-20 g dissolved in 100 ml MilliQ water, made up in amber glassware and stored at room temperature.

10. 10M Sodium hydroxide (BDH)-40 g dissolved in 100 ml MilliQ water (care should be exercised when preparing this solution as this reaction is exothermic), made up in “safebreak” glassware and stored at room temperature.

11. Hepatic or Supermix microsomes stored at −80° C. should be defrosted immediately prior to use, kept on ice and dispensed.

12. MilliQ water.

13. Thermostatically controlled shaking water bath set to give a temperature in the incubation of approx 37° C.

14. Reagent for termination of incubation (typically organic solvent, acid or base).

### Methodology for In Vitro Rate Determination Using Hepatic & Supermix Microsomes

The method outlined below is for a total incubation volume of 1.5 ml.

1. The following mixture is prepared in a test tube:

| | | | Volume added |
|---|---|---|---|
| | Stock | Concentration | (for 1.5 ml |
| Reagent | concentration | in incubation | incubation) |
| Phosphate | 200 mM | 50 mM | 375 μl |
| buffer pH 7.4 | | | |
| MgCl₂ | 0.1 M | 5 mM | 75 μl |
| Isocitric acid | 0.1 M | 5 mM | 75 μl |
| Isocitric | on bottle | 1 unit per ml* | see below* |
| dehydrogenase | | | |

*This volume is calculated for each new batch of isocitric dehydrogenase
e.g.
Protein concentration = 18 mg/ml
Enzyme activity = 3.3 units/mg
therefore Specific activity = 3.3 × 18 units/ml = 59 units/ml
For a 1.5 ml incubation 1.5 units of enzyme activity are required =
$\frac{1.5}{59} \times 1000 = 25.4 μ l .$

2. Defrost microsomes at room temperature and add sufficient microsomes to give a final concentration of 0.5 nmol cytochrome P450/ml of incubation e.g. for a 1.5 ml incubation, the volume of microsomes to be added is:

$$\frac{ \begin{matrix}P 450 concentration required in incubation \times \\ incubation volume\end{matrix}}{cytochrome P 450 concentration in microsomal prep .}$$

3. Add sufficient MilliQ water to give a total incubation volume of 1.425 ml.
4. Remove 237.5 μl of incubation mix and place in test tube for PNA positive control. Add 2.5 μl of PNA solution, whirlimix, and put tube into a rack in the thermostatically controlled shaking water bath

5. Remove 100 μl for no substrate control and dispense in test tube. Place test tube in a rack in the thermostatically controlled shaking water bath.

6. Add substrate to the incubation. The substrate should be at an initial concentration of 1 μM. The volume of substrate required in the remaining 1.162.5 ml incubation is calculated as follows:

$$\frac{RMN \times incubation vol . \times initial conc . in incubation}{1000 \times stock substrate solution conc .}$$

N.B. The volume of organic solvent added should not exceed 0.1% of the total incubation volume.

7. Remove 100 μl of incubation mix into test tube for no cofactor control. Whirlimix and put into a rack in the thermostatically controlled shaking water bath.

8. Pre-incubate the tube containing the incubation mix, also positive control and no cofactor tubes in the thermostatically controlled shaking water bath set at 37° C. for approx 5 min.

9. Add NADP to initiate reaction (75 μl to each 1.162.5 ml incubation mix, 12.5 μl to positive control tube and 5 μl to no substrate tube) and take first time point immediately. The PNA positive control, no cofactor control and no substrate tubes are incubated for the total incubation time.

10. Remove 100 μl aliquots up to 9 different sampling points from 0 to 60 min (usually 0, 3, 5, 10, 15, 20, 30, 45 & 60 min) and terminate reaction. Longer incubation times can be used, but, after 120 min the microsomes deteriorate. The reaction may be terminated by addition of organic solvent, acid or base. At the end of the incubation process the no cofactor and no substrate controls in a similar manner i.e. terminate with the same reagent.

11. PNA positive control procedure:
After the final sample has been taken, remove the positive control and add 1 ml 20% TCA to this tube. Also prepare a tube containing 250 μl of a PNP standard at 50 μM, and add 1 ml 20% TCA. Whirlimix both tubes and leave for approx 5 min to allow the protein to precipitate.
Centrifuge both tubes for approx 5 min in an instrument set at 3500 rpm. Remove 1 ml of supernatant and place into clean test tubes, discard the remainder.
Add 1 ml 10M NaOH to the supernatant, whirlimix, and leave to stand for approx 5 min. Blank spectrophotometer with distilled water at 400 nm then measure absorbance of the PNP standard against distilled water. The microsomal 4-nitroanisole O-demethylase activity is calculated as follows:

### Calculation of Results

$$\frac{\begin{matrix}Absorbance sample \times \\ nmoles P N P in standard ( ie 12.5 nmoles )\end{matrix}}{Absorbance P N P std \times 60 \times 0.125} = nmoles \text{/} min \text{/} nmol P 450$$

The activity value from the incubation MUST be equal to or greater than 85% of the mean value of the batch used for the incubation to be valid.
If this criteria is not met, then the incubation must be repeated.
   11. Analyse samples (including no cofactor and no substrate control) by a specific assay for the substrate to determine the disappearance kinetics.

### Analysis Of Data

Data obtained using the procedure described above can be quantified in terms of the substrates in vitro intrinsic clearance (Clint). Providing that the substrate concentration is below Km, the metabolism should be 1st order giving a log-linear plot of substrate disappearance with time.

The in vitro half-life of the substrate can be determined by plotting the natural logarithm (In) of a measure of relative substrate concentration (e.g. drug/internal standard ratio) against time and fitting the line of best fit to this data. The gradient of this line is the first order rate constant (k) for the substrate disappearance and is determined by regression analysis. This rate constant can be converted to the half-life according to the following equation:—

$$in vitro half \text{-} life ( {t}_{1 / 2} ) = - \frac{Ln 2}{k}$$

Alternatively the rate constant can be converted to an intrinsic clearance (Clint) according to the following equation:—

Clint(μl/min/mg)=(k/protein concentration in incubation(mg/ml))*1000

Preferably the compounds of the present invention exhibit a clearance, as determined by the above assay, expressed as a value of lower than about 200 μL/min/mg, more preferably lower than 100 μL/min/mg, yet more preferably lower than about 50 μL/min/mg and more preferably still lower than about 20 μL/min/mg. Using this assay, compounds according to the present invention which have been tested exhibit a clearance lower than 200 μL/min/mg.

Compounds of the invention intended for pharmaceutical use may be administered as crystalline or amorphous products. They may be obtained, for example, as solid plugs, powders, or films by methods such as precipitation, crystallization, freeze-drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

Accordingly the present invention provides for a pharmaceutical composition comprising a compound of formula (I) and a pharmaceutically acceptable diluent or carrier.

Any suitable route of administration may be employed for providing a mammal, especially a human with an effective dosage of a compound of the present invention. For example, oral (including buccal and sublingual administration), rectal, topical, parental, ocular, pulmonary, nasal, and the like may be employed. Dosage forms include tablets, troches, dispersions, suspensions, solutions, capsules, creams, ointments, aerosols, and the like. Preferably compounds of formula (I) are administered orally or intranasally.

The effective dosage of active ingredient employed may vary depending on the particular compound employed, the mode of administration, the condition being treated and the severity of the condition being treated. Such dosage may be ascertained readily by a person skilled in the art.

For the treatment of sexual dysfunction compounds of the present invention are given in a dose range of from about 0.001 milligram (mg) to about 1000 mg, preferably from about 0.001 mg to about 500 mg, more preferably from about 0.001 mg to about 100 mg, even more preferably from about 0.001 mg to about 50 mg and especially from about 0.002 mg to about 25 mg per kilogram of body weight, preferably as a single dose orally or as a nasal spray. For example, oral administration may require a total daily dose of from about 0.1 mg up to about 1000 mg, while an intravenous dose may only require from about 0.001 mg up to about 100 mg. The total daily dose may be administered in single or divided doses and may, at the physician's discretion, fall outside of the typical range given herein.

When treating obesity, in conjunction with diabetes and/or hyperglycemia, or alone, generally satisfactory results are obtained when the compounds of the present invention are administered at a daily dosage of from about 0.0001 mg to about 1000 mg, preferably about 0.001 mg to about 500 mg, more preferably about 0.005 mg to about 100 mg and especially about 0.005 mg to about 50 mg per kilogram of animal body weight, preferably given in a single dose or in divided doses two to six times a day, or in sustained release form. In the case of a 70 kg adult human, the total daily dose will generally be from about 0.7 mg up to about 3500 mg. This dosage regimen may be adjusted to provide the optimal therapeutic response.

When treating diabetes mellitus and/or hyperglycemia, as well as other diseases or disorders for which compounds of formula I are useful, generally satisfactory results are obtained when the compounds of the present invention are administered at a daily dosage of from about 0.001 mg up to about 100 mg per kilogram of animal body weight, preferably given in a single dose or in divided doses two to six times a day, or in sustained release form. In the case of a 70 kg adult human, the total daily dose will generally be from about 0.07 mg up to about 350 mg. This dosage regimen may be adjusted to provide the optimal therapeutic response.

These dosages are based on an average human subject having a weight of about 65 kg to 70 kg. The physician will readily be able to determine doses for subjects whose weight falls outside this range, such as infants, the elderly and the obese.

The compounds of the invention may be administered orally. Oral administration may involve swallowing, so that the compound enters the gastrointestinal tract, and/or buccal, lingual or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet. may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 wt % to 5 wt % of the tablet, and glidants may comprise from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 wt % to 10 wt %, preferably from 0.5 wt % to 3 wt % of the tablet.

Other possible ingredients include anti-oxidants, colourants, flavouring agents, preservatives and taste-masking agents.

Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula I, a film-forming polymer, a binder, a solvent, a humectant, a plasticiser, a stabiliser or emulsifier, a viscosity-modifying agent and a solvent. Some components of the formulation may perform more than one function.

The compound of formula I may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula I may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14 by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a suspension or as a solid, semi-solid, or thixotropic liquid for administration as an implanted depot providing modified release of the active compound. Examples of such formulations include drug-coated stents and semi-solids and suspensions comprising drug-loaded poly(d/-lactic-coglycolic)acid (PGLA) microspheres.

The compounds of the invention may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958 by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose, blisters and cartridges for use in an inhaler or insufflator may be formulated to contain a powder mix of the compound of the invention, a suitable powder base such as lactose or starch and a performance modifier such as /-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 0.001 mg to 10 mg of the compound of formula (I). The overall daily dose will typically be in the range 0.001 mg to 40 mg which may be administered in a single dose or, more usually, as divided doses throughout the day.

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline. Other formulations suitable for ocular and aural administration include ointments, gels, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For the avoidance of doubt, references herein to “treatment” include references to curative, palliative and prophylactic treatment.

### Experimental

The invention is illustrated by the following non-limiting examples in which the following abbreviations and definitions are used:

APCI atmospheric pressure chemical ionisation mass spectrum
[α]_{D} specific rotation at 589 nm.
Arbocel® filter agent
br broad
δ chemical shift

### d Doublet

dd double doublet
EI electrospray ionisation

### Ex Example

GC-MS gas chromatography mass spectrometry
HPLC high performance liquid chromatography
HRMS high resolution mass spectrum
LC-MS liquid chromatography mass spectrometry
LRMS low resolution mass spectrum

### m Multiplet

m/z mass spectrum peak
NMR nuclear magnetic resonance

### Prec Precursor

### Prep Preparation

psi pounds per square inch

### q Quartet

### Singlet

### t Triplet

tic thin layer chromatography

For synthetic convenience whilst in many instances compounds have been initially isolated in their free-base form, these have often been converted to their corresponding hydrochloride salts for analytical identification purposes. For the avoidance of doubt, both the free-base and HCl salt forms are considered provided herein.

### EXAMPLES

### Example I

### (3S,4R)-1-{[(3S,4R)-1-tert-butyl-4-(2,4-difluorophenyl)pyrrolidin-3-yl]carbonyl}-3-methyl-4-phenylpiperidin-4-ol hydrochloride

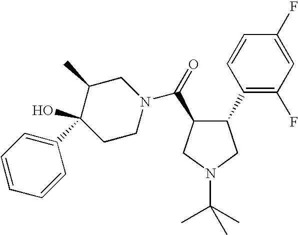

1-Propylphosphonic acid cyclic anhydride (50% in ethyl acetate, 0.37 mL, 0.62 mmol) was added to a mixture of (3S,4R)-3-methyl-4-phenylpiperidin-4-ol (synthesised according to J. Med. Chem. 1991, 34, 194) (100 mg, 0.52 mmol), triethylamine (0.22 mL, 1.56 mmol) and the acid of preparation 5 (200 mg, 0.62 mmol) in dichloromethane (5 mL) and the mixture was stirred at room temperature for 16 hours. Saturated aqueous sodium hydrogen carbonate solution (20 mL) was added to the reaction mixture and this was then extracted with dichloromethane (2×20 mL). The combined organic extracts were dried (MgSO₄), filtered and evaporated. The residue was purified by column chromatography (silica) eluting with dichloromethane/methanol (100% dichloromethane increasing polarity to 10% methanol in dichloromethane) to give the title compound as a white foam (203 mg, 86%). This was taken up in dichloromethane (3 mL) and converted to the hydrochloride salt by the addition of 2M ethereal HCl (2 mL). The solvent was removed in vacuo, the residue was taken up in dichloromethane and the salt was precipitated by the addition of pentane. The supernatant was removed and the solid was dried in vacuo to give the title compound (202 mg). ¹H NMR (CDCl₃, 400 MHz) δ 0.55-0.59 (m, 3H), 0.71 (dt 1H), 1.25-2.01 (m, 13H), 2.55-3.00 (m, 1H), 3.35-3.77 (m, 5H) 4.00-4.55 (m, 3H) 6.79-6.88 (m, 1H), 6.93-7.00 (m, 1H), 7.10-7.11 (d, 1H), 7.22-7.37 (m, 4H), 7.93-8.07 (m, 1H), 12.75 (br, s, 1H); LRMS (APCI⁺) 457 [MH⁺]; [α]_{D}25=−80.8 (c=0.25, MeOH).

### Examples 2-17

The following compounds of formula Ii, i.e. compounds of general formula I where n=1 and R⁷=2,4-difluorophenyl, were prepared by the method described for Example 1 starting from the appropriate amine and acid precursors, as indicated. In some cases alternative coupling conditions were used, wherein a solution of the amine and the acid was treated with 1-(3-dimethylaminopropyl)-3-ethyl-carbodiimide hydrochloride (EDCI), triethylamine or N-methylmorpholine and 1-hydroxybenzotriazole hydrate (HOBt) in dimethylformamide (DMF), tetrahydrofuran (THF), dichloromethane (DCM) or ethyl acetate at room temperature. In some cases the desired product was isolated and characterised as the free base rather than the hydrochloride salt.

(li)

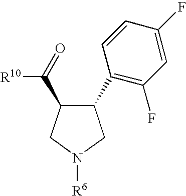

| | | | Prec. | Prec. | |
|---|---|---|---|---|---|
| Ex. | R⁶ | R¹⁰ | acid | amine | Data |
| 2 | 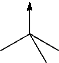 | 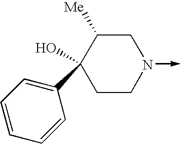 | Prep. 5 | Ref. a | 1H NMR (CDCl3, 400 MHz) δ 0.38 and 0.63(2xd, 3H) 0.75-0.85 (m, 1H), 1.50-2.03 (m,12H), 2.64-3.12 (m, 2H), 3.43-3.78 (m, 5H),4.08-4.54 (m, 3H), 6.80-6.90 (m, 1H), 6.95-7.03 (m, 1H) 7.12 (d, 1H), 7.22-7.38 (m,4H), 7.93-7.99 and 8.13-8.18 (2xm, 1H),12.85 (br, s, 1H); LRMS (APCl+) 457 [MH+];[α]_{D}²⁵ = −32.6 (c = 0.24, MeOH) |
| 3 | 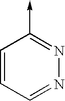 | 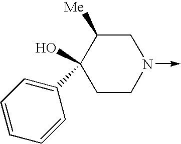 | Prep.11 | Ref. b | 1H NMR (CD3OD, 400 MHz) δ 0.57 and0.62 (2xd, 3H), 0.88-0.97 (m, 1H), 1.45-1.48and 1.64-1.71 and 1.92-2.09 (3xm, 3H),2.77-2.83 and 2.97-3.06 and 3.47-3.54(3xm, 2H), 3.74-4.50 (m, 8H), 7.03-7.24 (m,4H), 7.30-7.36 (m, 2H), 7.44-7.46 and 7.50-7.56 and 7.61-7.67 (3xm, 2H), 7.76-7.82 (m,1H), 7.88-7.92 (m, 1H), 8.56 (d, 1H); LRMS(APCl+) 479 [MH+]; [α]_{D}²⁵ = −57.6 (c = 0.25,MeOH) |
| 4 | 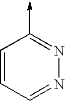 | 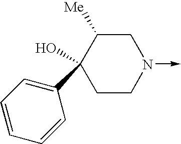 | Prep.11 | Ref. a | ¹H NMR (CD₃OD, 400 MHz) δ 0.45 and 0.62(2xd, 3H), 0.81-0.91 (m, 1H), 1.61-1.74 and2.00-2.06 (2xm, 2H), 2.74-2.80 and 3.02-3.28 (2xm, 3H), 3.68-4.50 (m, 8H), 7.01-7.65 (m, 8H), 7.70-7.73 (m, 1H), 7.84-7.88(m, 1H), 8.55 (d, 1H); LRMS (APCl⁺) 479[MH⁺]; [α]_{D}²⁵ = −13.9 (c = 0.26, MeOH) |
| 5 | 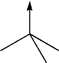 | 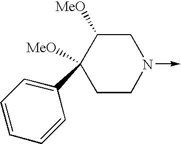 | Prep. 5 | Prep.18 | ¹H NMR (CD₃OD, 400 MHZ) δ 1.49 (s, 9H),1.84-1.92, 2.02-2.14 and 2.30-2.37 (3 × m,3H), 2.76-4.32 (m, 16H), 7.01-7.13 (m, 2H),7.27-7.44 (m, 5H), 7.60 (m, 1H); LRMS(APCl⁺) 487 [MH⁺]; [a]_{D}²⁵ = −29.4 (c = 0.27,MeOH) |
| 6 | 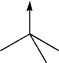 | 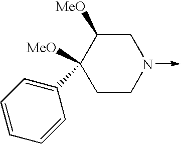 | Prep. 5 | Prep.21 | ¹H NMR (CD₃OD, 400 MHz) δ 1.48 (s, 9H),1.85 (m, 1H), 2.08 (m, 1H), 2.86-4.50 (m,17H), 7.05-7.44 (m, 7H), 7.65 (m, 1H);LRMS (APCl⁺) 487 [MH⁺]; [α]_{D}²⁵ = −29.5 (c =0.32, MeOH) |
| 7 | 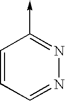 | 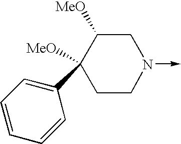 | Prep.11 | Prep.18 | ¹H NMR (CD₃OD, 400 MHz) δ 1.73-1.83,1.99-2.16 and 2.43-2.48 (3 × m, 3H), 2.94-3.43 (m, 8H), 3.69-3.95 (m, 3H), 4.09-4.35(m, 5H), 6.99-7.10 (m, 2H), 7.27-7.59 (m,6H), 7.78 (m, 1H), 7.91 (dd, 1H), 8.56 (dd,1H); LRMS (APCl⁺) 509 [MH⁺]; [α]_{D}²⁵ = −21.1(c = 0.27, MeOH) |
| 8 | 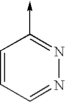 | 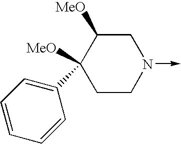 | Prep.11 | Prep.21 | ¹H NMR (CD₃OD, 400 MHz) δ 1.94 (m, 1H),212.12 (m, 1H), 2.94-3.43 (m, 9H), 3.71-3.98(m, 3H), 4.09-4.29 (m, 4H), 4.47 (m, 1H),7.02-7.14 (m, 2H), 7.22-7.63 (m, 6H), 7.76(m, 1H), 7.90 (m, 1H), 8.56 (m, 1H); LRMS(APCl⁺) 509 [MH⁺]; [α]_{D}²⁵ = −18.2 (c = 0.35,MeOH) |
| 9 | 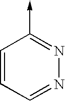 | 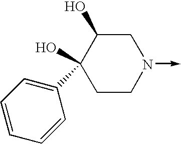 | Prep.11 | Prep.22 | ¹H NMR (CD₃OD, 400 MHz) δ 0.88, 1.52,1.76 and 1.93 (4 × m, 2H), 2.88-3.17 and3.40-3.58 (2 × m, 3H), 3.76-3.98 (m, 3H),4.10-4.25 (m, 4H), 4.35 and 4.52 (2 × m,1H), 7.00-7.14 (m, 2H), 7.20-7.25 (m, 3H),7.30-7.36 (m, 2H), 7.52 (m, 1H), 7.59 (m,1H), 7.75 (m, 1H), 7.88 (dd, 1H); LRMS(APCl⁺) 481 [MH⁺]; [α]_{D}²⁵ = −64.3 (c = 0.38,MeOH) |
| 10 | 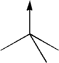 | 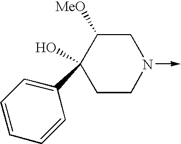 | Prep. 5 | Prep.24 | ¹H NMR (CD₃OD, 400 MHz) δ 1.51 (s, 9H),1.59-1.68 (m, 1H), 1.90-1.97 and 2.45-2.53(2x br m, 1H), 2.83-4.10 (m, 13H), 4.35-4.38and 4.59-4.62 (2xm, 1H), 7.04-7.25 and7.31-7.35 and 7.47-7.49 (3xm, 7H), 7.56-7.60 and 7.68-7.74 (2xm, 1H); LRMS(APCl⁺) 473 [MH⁺]; [α]_{D}²⁵ = −25.0 (c = 0.22,MeOH) |
| 11 | 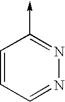 | 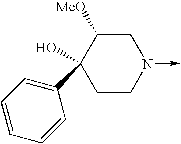 | Prep.11 | Prep.24 | ¹H NMR (CD₃OD, 400 MHz) δ 1.54-1.75 and1.94-2.01 and 2.60-2.64 (3xm, 3H), 2.89-3.06 (m, 4H), 3.20-4.24 (m, 9H), 4.36-4.41and 4.61-4.56 (2xm, 1H), 7.01-7.08 and7.12-7.26 and 7.31-7.37 and 7.49-7.52 and7.65-7.75 (4xm, 8H), 7.72-7.75 (m, 1H),7.86-7.90 (m, 1H) 8.57 (dd, 1H); LRMS(APCl⁺) 495 [MH⁺]; [α]_{D}²⁵ = −8.1 (c = 0.25,MeOH) |
| 12 | 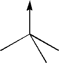 | 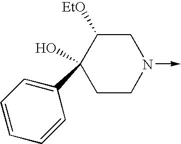 | Prep. 5 | Prep.26 | ¹H NMR (CD₃OD, 400 MHz) δ 0.77 and 0.87(2xt, 3H), 1.50 (s, 9H), 1.60-1.71 and 1.95-2.02 and 2.55-4.05 (3xm, 13H), 4.37-4.40and 4.54-4.57 (2xm, 1H), 7.03-7.34 (m, 6H),7.48 (d, 1H), 7.55-7.61 and 7.68-7.73 (2xm,1H); LRMS (APCl⁺) 487 [MH⁺]; [α]_{D}²⁵ = −27.1(c = 0.28, MeOH) |
| 13 | 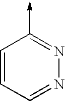 | 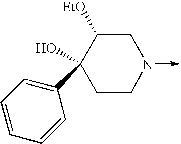 | Prep.11 | Prep.26 | ¹H NMR (CO₃OD, 400 MHz) δ 0.82 and 0.88(2xt, 3H), 1.57-1.77 and 1.97-2.05 (2xm,2H), 2.65-2.69 and 2.76-2.83 and 2.90-3.14(3xm, 3H), 3.20-3.48 and 3.59-3.63 and3.78-4.23 (3xm, 9H), 4.38-4.43 and 4.55-4.65 (2xm, 1H), 7.01-7.26 (m, 4H), 7.31-7.36 (m, 2H), 7.47-7.52 and 7.61-7.67 (2xm,2H), 7.74-7.78 (m, 1H), 7.89 (dd, 1H), 8.56(d, 1H); LRMS (APCl⁺) 509 [MH⁺];[α]_{D}²⁵ = −9.1 (c = 0.31, MeOH) |
| 14 | 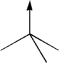 | 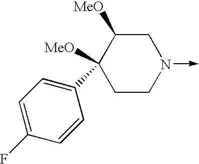 | Prep. 5 | Prep.29 | ¹H NMR (CD₃OD, 400 MHz) δ 1.49 (s, 9H),1.82-2.16 (m, 2H), 2.80-3.14 (m, 8H), 3.45-4.08 (m, 8H), 4.32 and 4.50 (2 × m, 1H),7.06-7.28 (m, 5H), 7.41-7.46 and 7.56-7.68(2 × m, 2H); LRMS (APCl⁺) 505 [MH⁺];[α]_{D}²⁵ = −31.9 (c = 0.25, MeOH) |
| 15 | 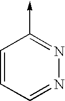 | 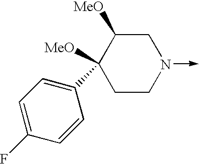 | Prep.11 | Prep.29 | ¹H NMR (CD₃OD, 400 MHZ) δ 1.00-1.11and 1.91-2.18 (2 × m, 2H), 2.94-3.42 (m,9H), 3.71-4.32 and 4.48-4.54 (2 × m, 8H),7.02-7.14, 7.23-7.29 and 7.44-7.62 (3 × m,7H), 7.76 (m, 1H), 7.91 (m, 1H), 8.56 (m,1H); LRMS (APCl⁺) 527 [MH⁺]; [α]_{D}²⁵ = −17.6 (c = 0.23, MeOH) |
| 16 | 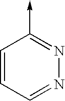 | 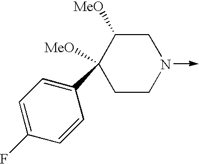 | Prep.11 | Prep.32 | ¹H NMR (CD₃OD, 400 MHz) δ 1.76, 2.01-2.17, 2.39 (3 × m, 2H), 2.91-3.45 (m, 9H),3.66-400 (m, 3H), 4.04-4.26 (m, 4H), 4.32(m, 1H), 6.97-7.16 (m, 4H), 7.32 (m, 1H),7.42-7.61 (m, 2H), 7.72-7.84 (m, 1H), 7.91(m, 1H), 8.57 (m, 1H); LRMS (APCl⁺) 527[MH⁺]; [α]_{D}²⁵ = −18.9 (c = 0.20, MeOH) |
| 17 | 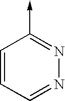 | 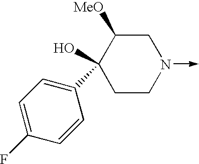 | Prep.11 | Prep.34 | ¹H NMR (CD₃OD, 400 MHz) δ1.04, 1.54, 1.70, 1.90 (4 × m, 2H), 2.82-3.25(m, 5H), 3.38-3.93 (m, 4H), 3.96-4.24 (m,4H), 4.35, 4.70 (2 × m, 1H), 6.93-7.12 (m,5H), 7.23-7.33 (m, 1H), 7.37-7.62 (m, 3H),8.43-8.50 (m, 1H); LRMS (ES⁺) 513 [MH⁺] |
| 18 | 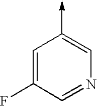 | 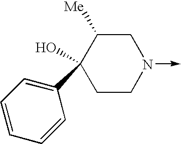 | Prep.55 | Ref. a | LRMS (APCl+) 496 [MH+] |
| 19 | 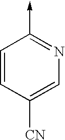 | 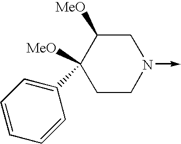 | Prep.57 | Prep21 | LRMS (APCl+) 533 [MH+] |
| 20 | 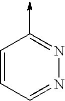 | 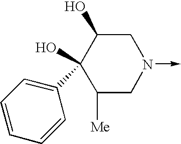 | Prep.11 | Ref. c | LRMS (APCl⁺) 495 [MH⁺] |
| 21 | 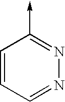 | 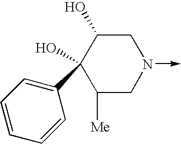 | Prep.11 | Ref. c | LRMS (APCl⁺) 495 [MH⁺] |
| 22 | 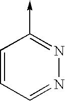 | 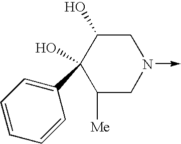 | Prep.11 | Ref. c | LRMS (El⁺) 495 [MH⁺] |
| 23 | 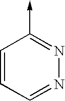 | 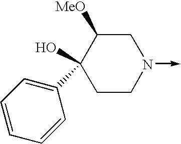 | Prep.11 | Prep.41 | LRMS (APCl⁺) 495 [MH⁺] |
| 24 | 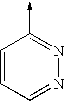 | 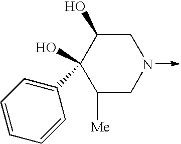 | Prep.11 | Ref. c | LRMS (APCl⁺) 495 [MH⁺] |
| 25 | 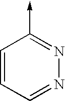 | 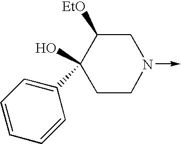 | Prep.11 | Prep.42 | LRMS (APCl⁺) 509 [MH⁺] |
| 26 | 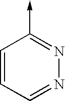 | 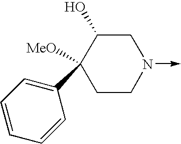 | Prep.11 | Prep.39 | LRMS (APCl⁺) 495 [MH⁺] |
| 27 | 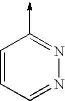 | 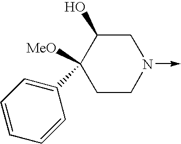 | Prep.11 | Prep.38 | LRMS (APCl⁺) 495 [MH⁺] |
| 28 | 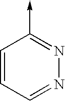 | 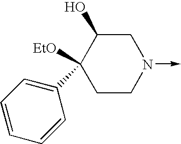 | Prep.11 | Prep.40 | LRMS (APCl⁺) 509 [MH⁺] |
| 29 | 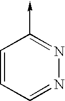 | 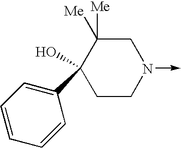 | Prep.11 | Prep.45b | LRMS (APCl⁺) 493 [MH⁺]HRMS 493.2409 [MH+] |
| 30 | 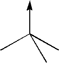 | 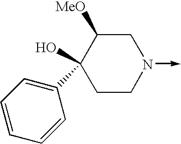 | Prep. 5 | Prep.41 | LRMS (APCl⁺) 473 [MH⁺] |
| 31 | 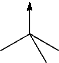 | 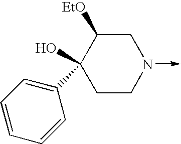 | Prep. 5 | Prep.42 | LRMS (APCl⁺) 487 [MH⁺] |
| 32 | 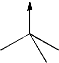 | 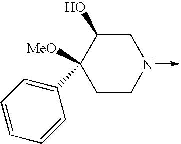 | Prep. 5 | Prep.38 | LRMS (APCl⁺) 473 [MH⁺] |
| 33 | 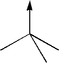 | 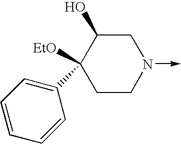 | Prep. 5 | Prep.40 | LRMS (APCl⁺) 487 [MH⁺] |
| 34 | 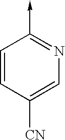 | 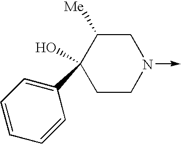 | Prep.57 | Ref. a | LRMS (APCl⁺) 503 [MH⁺] |
| 35 | 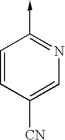 | 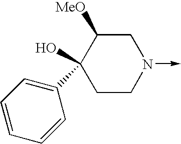 | Prep.57 | Prep.41 | LRMS (APCl⁺) 520 [MH⁺] |
| 36 | 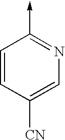 | 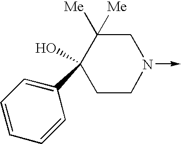 | Prep.57 | Prep.45b | LRMS (APCl⁺) 517 [MH⁺] |
| 37 | 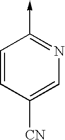 | 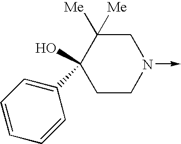 | Prep.57 | Prep.45a | LRMS (APCl⁺) 517 [MH⁺] |

Ref. a—(3R,4S)-3-methyl-4-phenylpiperidin-4-ol was synthesised according to J. Med. Chem. 1991, 34, 194
Ref. b—(3S,4R)-3-methyl-4-phenylpiperidin-4-ol was synthesised according to J. Med. Chem. 1991, 34, 194
Ref. c—The 4 diastereoisomers of 3,4-dihydroxy-5-methyl-4-phenylpiperidine were prepared by dihydroxylation of the alkene of preparation 59 according to the methods of preparations 16 and 19 followed by deprotection according to the method of preparation 18. The compounds of examples 20, 21, 22, 24 were separated by a combination of column chromatography and chiral phase HPLC.

### Example 19

### Alternative Method

### 5-[(3R,4S)-3-(2,4-D-difluorophenyl)-4-{[(3S4S)-3,4-dimethoxy-4-phenylpiperidin-1-yl]carbonyl}pyrrolidin-1-yl]pyridine-2-carbonitrile

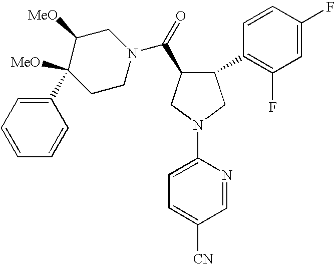

To a solution of the product from preparation 15 (50 mg, 0.1 mmol) in degassed toluene (5 mL) was added sodium tert-butoxide (16 mg, 0.22 mmol), 2-bromo-5-cyanopyridine (68 mg, 0.37 mmol), 2,2′-bis(diphenylphosphino)-1,1′-binaphthyl (13 mg, 0.02 mmol), palladium dibenzylideneacetone (5.5 mg, 0.01 mmol) and the reaction mixture was heated to 80° C. for 6 h. Further palladium dibenzylideneacetone (5.5 mg) and 2,2′-bis(diphenylphosphino)-1,1′-binaphthyl (13 mg) were added and the mixture was heated at reflux overnight. The reaction mixture was filtered, washed with toluene and the solvent was removed. The residual oil was purified by column chromatography (silica) eluting with dichloromethane:methanol (1:0 to 95:5) to afford the title compound as a yellow foam (35 mg, 61%). ¹H NMR (CD₃OD, 400 MHz) δ 1.2 (m, 1H), 2.0 (m, 2H), 2.95 (m, 1H), 3.1 (m, 6H), 3.4 (m, 2H), 3.5-4.4 (m, 6H), 4.47 (d, 1H), 7.0 (m, 3H), 7.2-7.9 (m, 7H), 8.05 (br, 1H); LRMS (APCI⁺) 533 [MH⁺].

### Example 38

### (3S,4S)-1-{[(3S,4R)-4-(2,4-Difluorophenyl)-1-propionylpyrrolidin-3-yl]carbonyl}-3,4-dimethoxy-4-phenylpiperidine

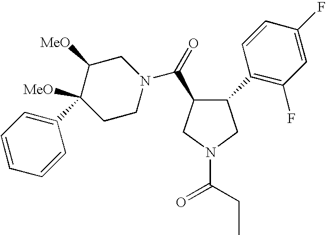

The hydrochloride salt of the amine of preparation 15 (100 mg, 0.21 mmol) was suspended in dichloromethane (2 mL) and triethylamine (90 μL, 0.64 mmol) was added to give a clear solution. Propionyl chloride (27 μl, 0.32 mmol) was then added and the reaction mixture was stirred at room temperature for 16 hours. The reaction was quenched by the addition of saturated aqueous sodium hydrogen carbonate solution (10 mL) and the mixture was extracted with ethyl acetate (10 mL). The organic layer was washed with brine, dried (MgSO₄) and evaporated. The residue was purified by column chromatography (silica) eluting with dichloromethane/methanol/ammonia (99:1:0.1 increasing polarity to 98:2:0.2) to give the title compound as a white foam (76 g, 74%). ¹H NMR (CDCl₃, 400 MHz) δ1.00-1.50 (m, 4H), 1.83-2.19 (m, 1H), 2.26-2.38 (m, 2H), 2.82-3.19 (m, 8H), 3.26-4.20 (m, 8H), 4.40-4.61 (m, 1H), 6.81-6.96 (m, 2H), 7.19-7.42 (m, 6H); LRMS (APCI⁺) 487 [MH⁺]; [α]_{D}²⁵=−25.4 (c=0.18, MeOH).

### Example 39

### Methyl (3R,4S)-3-(2,4-difluorophenyl)-4-{[(3S,4S)-3,4-dimethoxy-4-phenylpiperidin-1-yl]carbonyl}pyrrolidine-1-carboxylate

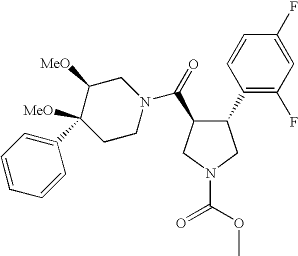

The title compound was prepared from the hydrochloride salt of the amine of preparation 15 according to the method of Example 38 using methyl chloroformate instead of propionyl chloride. ¹H NMR (CDCl₃, 400 MHz) δ0.82-1.39 (m, 1H), 1.91-2.19 (m, 2H), 2.81-3.28 (m, 7H), 3.28-4.05 (m, 1H), 4.40-4.53 (m, 1H), 6.78-6.93 (m, 2H), 7.18-7.43 (m, 6H); LRMS (APCI⁺) 489 [MH⁺]; [α]_{D}²⁵=−18.6 (c=0.16, MeOH).

### Example 40

### Ethyl (3R,4S)-3-(2,4-difluorophenyl)-4-{[(3S,4S)-3,4-dimethoxy-4-phenylpiperidin-1-yl]carbonyl}pyrrolidine-1-carboxylate

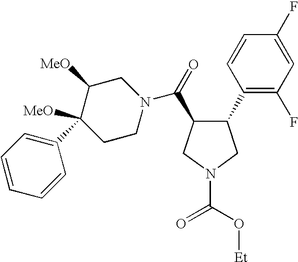

The title compound was prepared from the hydrochloride salt of the amine of preparation 15 according to the method of Example 38 using ethyl chloroformate instead of propionyl chloride. ¹H NMR (CDCl₃, 400 MHz) δ1.20-1.36 (m, 3H), 1.92-2.19 (m, 2H), 2.82-2.96 (m, 1H), 2.98-3.18 (m, 7H), 3.27-4.22 (m, 10H), 4.41-4.62 (m, 1H), 6.75-6.93 (m, 2H), 7.19-7.42 (m, 6H); LRMS (APCI⁺) 503 [MH⁺]; [α]_{D}²⁵=−25.4 (c=0.2, MeOH).

### Example 41

### (3S,4S)-1-{[(3S,4R)-4-(2,4-Difluorophenyl)-1-(tetrahydro-2H-pyran-4-yl)pyrrolidin-3-yl]carbonyl}-3,4-dimethoxy-4-phenylpiperidine hydrochloride

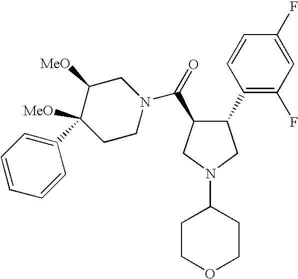

The hydrochloride salt of the amine of preparation 15 (100 mg, 0.21 mmol) was dissolved in ethanol (2 mL) with triethylamine (60 μL, 0.42 mmol) and stirred for 5 minutes. Tetrahydro-4H-pyran-4-one (30 μl, 0.32 mmol) was then added and the reaction mixture was stirred for a further 10 minutes before the addition of sodium triacetoxyborohydride (68 mg, 0.32 mmol). The reaction was stirred at room temperature for 16 hours and the solvent was then removed in vacuo. The residue was partitioned between water (15 mL) and ethyl acetate (20 mL) and the organic layer was washed with water (15 mL) and brine, dried (MgSO₄) and evaporated. The residue was purified by column chromatography (silica) eluting with dichloromethane/methanol/ammonia (99:1:0.1 increasing polarity to 97:3:0.3) to give the title compound as a colourless oil. This was dissolved in dichloromethane (2 mL) and 4M HCl in dioxane was added to form the hydrochloride salt. The solvent was removed in vacuo and the residue was azeotroped with toluene (10 mL) and then dichloromethane (2 mL) to give the title compound as a white foam (95 mg, 82%). ¹H NMR (CDCl₃, 400 MHz) δ1.52-1.70 (m, 2H), 1.73-2.16 (m, 4H), 2.35-2.43 (m, 1H), 2.63-3.70 (m, 17H), 3.92-4.03 (m, 3H), 4.44-4.68 (m, 1H), 6.62-6.90 (m, 2H), 7.22-7.53 (m, 6H); LRMS (APCI⁺) 515 [MH⁺]; [α]_{D}²⁵=−23.6 (c=0.21, MeOH).

### Example 42

### 3-Chloro-6-{(3R,4S)-3-(2,4-difluorophenyl)-4-{[(3S,4S)-3,4-dimethoxy-4-phenylpiperidin-1-yl]carbonyl}pyrrolidin-1-yl]pyridazine

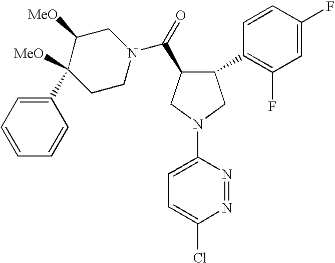

To a solution of product from preparation 15 (175 mg, 0.40 mmol) in tetrahydrofuran (2 mL) was added 2,5-dichloropyridazine (121 mg, 0.80 mmol), N,N-diisopropylethylamine (0.21 mL, 1.20 mmol) and the reaction mixture was heated at reflux overnight. The solvent was removed and the residue was partitioned between water and dichloromethane. The organic layer was separated, dried, concentrated in vacuo. The residual oil was purified by column chromatography eluting with pentane: ethyl acetate to afford the title compound as a white foam (250 mg). ¹H NMR (CD₃Cl, 400 MHz) δ 1.31-1.39 and 1.97-2.06 and 2.12-2.17 (3×m, 2H), 2.89-2.99 and 3.08-3.15 (2×m, 7.5H), 3.35-3.43 (m, 1H), 3.61-3.88 (m, 4H), 3.98-4.14 (m, 3H), 4.18-4.24 (q, 0.5H), 4.45 (br d, 0.5H), 4.58-4.62 (dd, 0.5H), 6.64-6.68 and 6.81-6.93 2×m, 3H), 7.20-7.42 (m, 7H); LRMS (APCI⁺) 543 [MH⁺], (ESI⁺) 543 [MH⁺].

### Example 43

### 6-(3R,4S)-3-(2,4-Difluorophenyl)-4-{[(3S,4S)-3,4-dimethoxy-4-phenylpiperidin-1-yl]carbonyl}pyrrolidin-1-yl]pyridazin-3(2H)-one

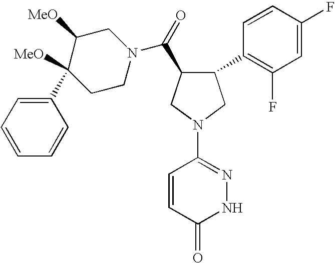

Degassed acetic acid was added to the product from example 42 at room temperature and the mixture was degassed for 5 min before being heated at reflux for 20 h. The reaction mixture purified by column chromatography (silica) eluting with dichloromethane:methanol:ammonia (1:0:0 to 95:5:0.5), followed by reverse phase HPLC to afford the title compound as a yellow solid (64 mg, 42%). ¹H NMR (CD₃OD, 400 MHz) δ 1.20-1.28 and 1.92-1.97 and 2.07-2.15 (3×m, 2H), 2.94-3.01 (m, 1H), 3.06-3.12 (m, 6H), 3.25-3.40 and 3.49-3.67 and 3.77-3.81 (3×m, 4H), 3.87-3.99 and 4.03-4.13 and 4.22-4.28 and 4.44-4.49 (4×m, 6H), 6.87-6.92 (m, 1H), 6.96-7.07 (m, 2H), 7.25-7.40 and 7.44-7.56 (2×m, 7H); LRMS (APCI⁺) 525 [MH⁺], (ESI⁺) 525 [MH⁺].

### Example 44

### 6-(3R,4S-3-(2,4-Difluorophenyl)-4-{[(3S,4S)-3,4-dimethoxy-4-phenylpiperidin-1-yl]carbonyl}pyrrolidin-1yl-3-methylpyridazin-3(2H)-one

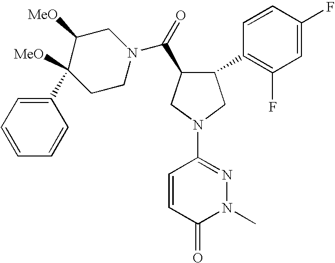

To a solution of the product from example 43 (45 mg, 0.09 mmol) in dimethylformamide (1 mL) was added potassium bis(trimethylsilyl)amide (19 mg, 0.10 mmol), lithium bromide (9 mg, 0.10 mmol) and methyl iodide (0.006 mL, 0.10 mmol) and the reaction mixture was stirred at room temperature overnight. Further methyl iodide (0.002 mL) was added and the mixture was stirred for another 2 h before being partitioned between water and ethyl acetate. The organic layer was dried, concentrated and the residue was purified by column chromatography (silica) eluting with dichloromethane:methanol:ammonia (1:0:0 to 95:5:0.5) to afford the title compound as a yellow oil (17 mg, 37%). ¹H NMR (CD₃OD, 400 MHz) δ 1.21-1.29 and 1.93-1.98 and 2.04-2.14 and 2.85-3.04 (4×m, 4H), 3.06-3.12 (m, 6H), 3.24-3.41 and 3.49-3.67 and 3.74-3.80 and 3.88-3.99 and 4.03-4.12 and 4.22-4.27 and 4.45-4.49 (7×m, 11H), 6.86-6.91 (m, 1H), 6.96-7.07 (m, 2H), 7.25-7.32 and 7.34-7.40 and 7.44-7.47 and 7.49-7.56 (4×m, 7H); LRMS (APCI⁺) 539 [MH⁺], (ESI⁺) 539 [MH⁺].

### Example 45

### 3-[(3R,4S)-3-(2,4-Difluorophenyl)-4-{[(3S,4S)-3,4-dimethoxy-4-phenylpiperidin-1-yl]carbonyl}pyrrolidin-1yl]-6-methoxypyridazine

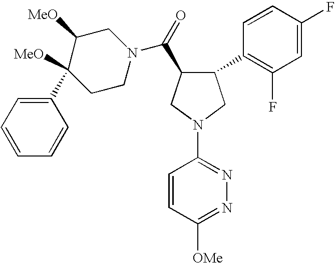

To a solution of the product from example 42 (52 mg, 0.1 mmol) in methanol (3 mL) was added sodium (25 mg), caesium fluoride (30 mg, 0.2 mmol) and the mixture was heated at 120° C. in a microwave for 5 h. The solvent was removed and the residue was partitioned between water and dichloromethane. The organic layer was dried, concentrated and the residue purified by reverse phase HPLC to afford the title compound. ¹H NMR (CD₃OD, 400 MHz) δ 1.21-1.30 and 1.94-1.99 and 2.04-2.16 and 2.93-3.00 (4×m, 3H), 3.05-3.13 (m, 6H), 3.34-3.41 and 3.55-3.60 and 3.64-3.75 and 3.80-3.85 (4×m, 4H), 3.94-3.97 and 3.99-4.04 and 4.07-4.19 and 4.23-4.29 and 4.47-4.51 (5×m, 8H), 6.96-7.10 and 7.25-7.57 (2×m, 10H); LRMS (APCI⁺) 539 [MH⁺]

### Example 46

### 6-[(3R,4S)-3-(2,4-Difluorophenyl)-4-{[(3S,4S)-3,4-dimethoxy-4-phenylpiperidin-1-yl]carbonyl}pyrrolidin-1-yl]pyridazin-3-carbonitrile

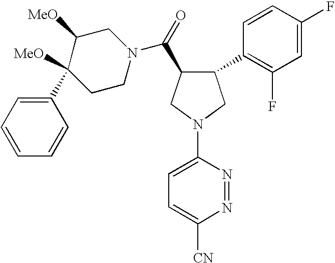

To a solution of the product from preparation 15 (87.0 mg, 0.19 mmol) in tetrahydrofuran (2 mL) was added 2-chloro-5-cyanopyridazine (52.0 mg, 0.37 mmol), N,N-diisopropylethylamine (0.1 mL, 0.56 mmol) and the reaction mixture was heated at reflux for 1.5 h. The solvent was removed and the residue was purified by column chromatography (silica) eluting with pentane:ethyl acetate to afford the title compound (84 mg, 84%). ¹H NMR (CD₃OD, 400 MHz) δ 1.15-1.22 and 1.28 and 1.73-1.82 (m, s, m, 1H), 1.95 and 2.04-2.13 and 2.48-2.51 and 2.60-2.62 (d, 3×m, 3H), 2.95-3.12 (m, 2×s, 7H), 3.33-3.45 (m, 1H), 3.70-3.93 and 4.03-4.33 and 4.47-4.51 (4×m, 7H), 6.98-7.11 (m, 3H), 7.24-7.32 (m, 2H), 7.35-7.40 (m, 2H), 7.45-7.59 (m, 2H), 7.68-7.72 (m, 1H); LRMS (APCI⁺) 534 [MH⁺], (ESI⁺) 534 [MH⁺].

### Examples 47-59

The following compounds of formula I, where n=1, were prepared by methods analogous to those used for examples 1-46. The desired products were isolated and characterised as either the free base or an appropriate salt, for example the hydrochloride salt.

(li)

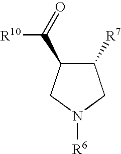

| Ex. | R⁶ | R⁷ | R¹⁰ | Data |
|---|---|---|---|---|
| 47 | 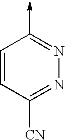 | 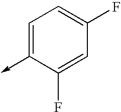 | 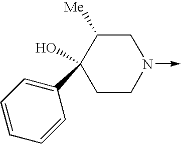 | LRMS (APCl⁺) 504 [MH⁺] |
| 48 | 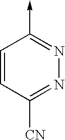 | 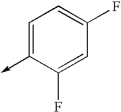 | 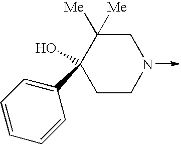 | LRMS (APCl⁺) 518 [MH⁺]HRMS 518.2359 [MH+] |
| 49 | 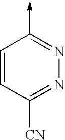 | 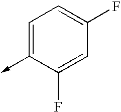 | 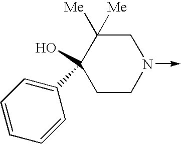 | LRMS (APCl⁺) 518 [MH⁺]HRMS 518.2363 [MH+] |
| 50 | 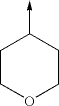 | 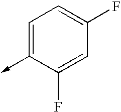 | 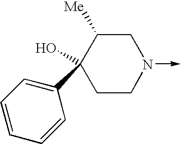 | LRMS (APCl⁺) 485 [MH⁺] |
| 51 | 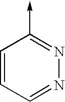 | 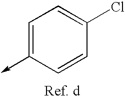 | 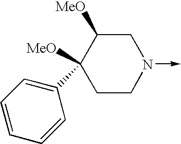 | LRMS (APCl+) 507 [MH+] |
| 52 | 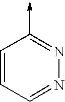 | 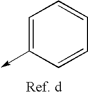 | 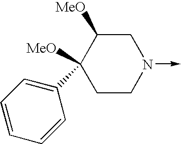 | LRMS (APCl+) 473 [MH+] |
| 53 | 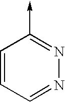 | 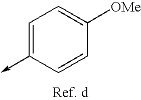 | 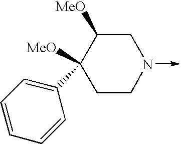 | LRMS (APCl+) 503 [MH+]HRMS 503.2642 [MH+] |
| 54 | 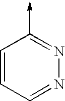 | 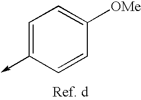 | 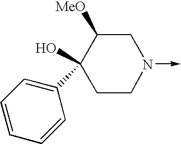 | LRMS (APCl+) 489 [MH+]HRMS 489.2489 [MH+] |
| 55 | 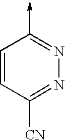 | 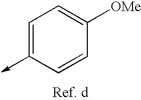 | 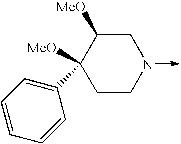 | LRMS (APCl+) 489 [MH+]HRMS 528.2603 [MH+] |
| 56 | 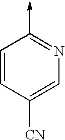 | 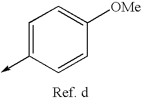 | 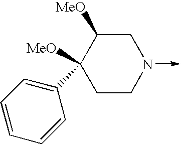 | LRMS (APCl+) 527 [MH+]HRMS 527.2635 [MH+] |
| 57 | 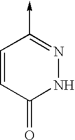 | 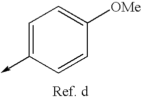 | 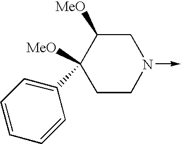 | LRMS (APCl+) 519 [MH+]HRMS 519.2584 [MH+] |
| 58 | 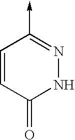 | 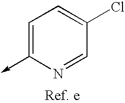 | 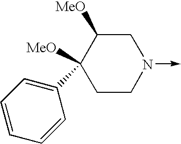 | LRMS (APCl+) 524 [MH+] |
| 59 | 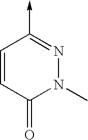 | 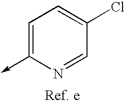 | 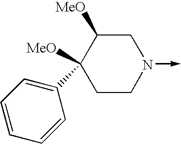 | LRMS (APCl+) 538 [MH+] |

Ref. d—Substituted pyrrolidines where R⁷ = substituted aryl were synthesised by methods analogous to those described in preparations 3 to 7, starting from the appropriate substituted cinnamic acids.
Ref. e—Compounds where R⁷ = 5-chloropyridin-2-yl were synthesised starting from the acid of preparation 67.

### Examples 60-64

The following compounds of formula Iii, i.e. compounds of general formula I where n=2 and R⁷=2,4-difluorophenyl, were prepared by the methods described above for examples 1-47 but starting from the piperidine of preparation 53. The desired products were isolated and characterised as either the free base or an appropriate salt, for example the hydrochloride salt.

(lii)

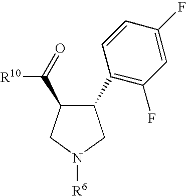

| Ex. | R⁶ | R¹⁰ | Data |
|---|---|---|---|
| 60 | H | 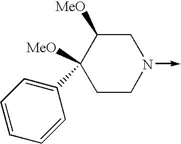 | LRMS (APCl+)445 [MH+] |
| 61 | 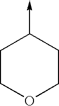 | 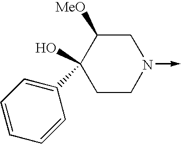 | LRMS (APCl+)515 [MH+] |
| 62 | 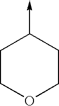 | 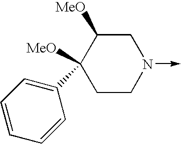 | LRMS (APCl+)530 [MH+] |
| 63 | 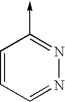 | 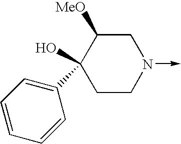 | LRMS (APCl+)509 [MH+] |
| 64 | 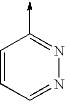 | 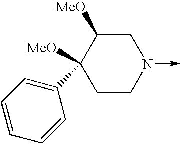 | LRMS (APCl+)523 [MH+] |

### Preparations

### Preparation 1

### 2-Methyl-N—[(trimethylsilyl)methyl]propan-2-amine

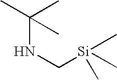

A procedure is given in J. Org. Chem. 53(1), 194, 1988 for the preparation of this intermediate. Alternative procedures are given below:

A solution of (chloromethyl)trimethylsilane (50 g, 408 mmol) and tert-butylamine (130 mL) under dry nitrogen was heated at 200° C. in a sealed tube for 18 hours before being quenched by the addition of 2M sodium hydroxide solution (700 mL). The resulting mixture was extracted with diethyl ether (3×100 mL) and the combined organic layers were distilled under dry nitrogen at 1 atmosphere to afford the title compound as a clear oil (62 g, 96%). ¹H NMR (CDCl₃, 400 MHz) δ 0.05 (s, 9H), 1.05 (s, 9H), 1.95 (s, 2H).

### Alternative Preparation:

(Chloromethyl)trimethylsilane (100 mL, 730 mmol) and tert-butylamine (250 mL, 2400 mmol) were placed in a sealed bomb and heated with vigorous stirring for 18 hours. On cooling to room temperature, the slurry of the hydrochloride salts produced and residual excess tert-butylamine were poured into 4M sodium hydroxide solution (500 mL) and stirred vigorously for 1 hour. The aqueous layer was separated and the organic layer was stirred vigorously with water (3×500 mL) (the excess tert-butylamine is very water soluble, the product is only sparingly soluble). The residual organic layer was dried over sodium sulfate to give essentially pure 2-methyl-N—[(trimethylsilyl)methyl]propan-2-amine (105.4 g), which was used without further purification.

### Preparation 2

### N-(Methoxymethyl)-2-methyl-N—[(trimethylsilyl)methyl]propan-2-amine

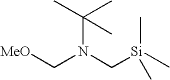

2-Methyl-N—[(trimethylsilyl)methyl]propan-2-amine (from preparation 1) (4.31 g, 27 mmol) was added to an ice-cooled mixture of methanol (1.29 mL, 31.8 mmol) and aqueous formaldehyde (37% w/v 2.49 mL, 33 mmol) over 45 minutes. The heterogeneous mixture was stirred at 0° C. for 2 hours and then solid potassium carbonate (325 mesh) (1.08 g, 13 mmol) was added and the mixture was stirred for 30 minutes at 0° C. The layers were separated and the aqueous phase was extracted with ethyl acetate (3×20 mL). The combined organic layers were dried over sodium sulfate, filtered, and evaporated under reduced pressure to give an 80:20 mixture of the title compound and unreacted tert-butyl[(trimethylsilyl)methyl]amine as a colourless oil (5.09 g). The mixture was used directly without further purification. ¹H NMR (CD₃OD, 400 MHz) δ 0.04 (s, 9H), 1.11 (s, 9H), 2.27 (s, 2H), 3.34 (s, 3H), 4.17 (s, 2H).

### Preparation 3

### (4S)-4-Benzyl-3-[(2E)-3-(2,4-difluorophenyl)prop-2-enoyl]-1,3-oxazolidin-2-one

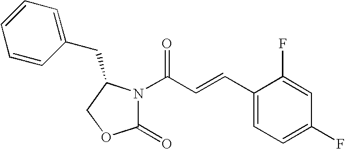

Oxalyl chloride (19 mL, 216 mmol) in dichloromethane (50 mL) was added dropwise to an ice-cooled stirred suspension of 2,4-difluorocinnamic acid (20.0 g, 108 mmol) in dichloromethane (400 mL) and N,N-dimethylformamide (0.4 mL) over 0.5 hours (waste gases from the reaction were scrubbed with a solution of concentrated sodium hydroxide). Once addition was complete, the reaction mixture was allowed to warm up to room temperature and was stirred at room temperature under nitrogen for 18 hours. The reaction mixture was then concentrated and azeotroped with dichloromethane (2×50 mL). The resulting acid chloride was dissolved in dichloromethane (50 mL) and this solution was added dropwise under nitrogen to a vigorously stirred suspension of lithium chloride (23.0 g, 540 mmol), triethylamine (76 mL, 540 mol) and (S)-(−)-4-benzyl-2-oxazolidinone (18.3 g, 103 mmol) in dichloromethane (400 mL) over 30 minutes. Once addition was complete, the reaction mixture was stirred at room temperature under nitrogen for 2.5 hours. The reaction mixture was diluted with dichloromethane (200 mL) and treated with a solution of 5% citric acid solution (500 mL). The organic layer was then separated and dried over magnesium sulfate. Filtration and evaporation of the dichloromethane gave the crude product as an orange oil. The crude material was dissolved in dichloromethane (100 mL) and the resulting solution was passed through a plug of silica, eluting with dichloromethane. The filtrate (1 L) was finally concentrated to afford 30.8 g of the product as a white solid. ¹H NMR (CDCl₃, 400 MHz) δ 2.85 (dd, 1H), 3.36 (dd, 1H), 4.22 (m, 2H), 4.80 (m, 1H), 6.90 (m, 2H), 7.68 (m, 5H), 7.68 (dd, 1H), 7.91 (d, 1H), 8.01 (dd, 1H); LRMS (APCI⁺) 344 [MH⁺].

### Preparation 4a

### (4S)-4-Benzyl-3-{[(3R,4S)-1-tert-butyl-4-(2,4-difluorophenyl)pyrrolidin-3-yl]carbonyl}-1,3-oxazolidin-2-one and

### Preparation 4b

### (4S)-4-Benzyl-3-{[(3S,4R)-1-tert-butyl-4-(2,4-difluorophenyl)pyrrolidin-3-yl]carbonyl}-1,3-oxazolidin-2-one

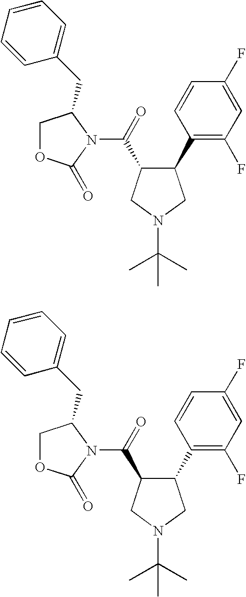

A stirred solution of (S)-4-benzyl-3-[3-(2,4-difluoro-phenyl)-acryloyl]-oxazolidin-2-one (from preparation 3) (1.70 g, 4.95 mmol) and N-(methoxymethyl)-2-methyl-N—[(trimethylsilyl)methyl]propan-2-amine (from preparation 2) (1.60 g, 5.94 mmol) in dichloromethane (15 mL) was treated with trifluoroacetic acid (0.075 mL, 1 mmol). The resulting mixture was stirred at room temperature under nitrogen for 4.5 hours. The reaction mixture was diluted with dichloromethane (50 mL) and treated with saturated aqueous sodium hydrogen carbonate solution (50 mL). The organic layer was separated and the aqueous layer was extracted with dichloromethane (50 mL). The organic fractions were combined and dried over magnesium sulfate. Filtration and evaporation of the dichloromethane gave the crude mixture of diastereoisomers.

Separation by column chromatography on silica gel with pentane:ethyl acetate 80/20 to 10/90 v/v, gradient elution, afforded firstly 0.74 g (1.67 mmol) of (4S)-4-benzyl-3-{[(3R,4S)-1-tert-butyl-4-(2,4-difluorophenyl)pyrrolidin-3-yl]carbonyl}-1,3-oxazolidin-2-one as a colourless oil, and then 0.82 g (1.85 mmol) of (4S)-4-benzyl-3-{[(3S,4R)-1-tert-butyl-4-(2,4-difluorophenyl)pyrrolidin-3-yl]carbonyl}-1,3-oxazolidin-2-one as a white solid.

(4S)-4-benzyl-3-{[(3R,4S)-1-tert-butyl-4-(2,4-difluorophenyl)pyrrolidin-3-yl]carbonyl}-1,3-oxazolidin-2-one-¹H NMR (CDCl₃, 400 MHz) δ 1.12 (s, 9H), 2.77 (dd, 1H), 2.85 (m, 1H), 3.25 (dd, 1H), 3.17-3.47 (m, 1H), 4.15 (m, 3H), 4.65 (m, 1H), 6.74 (t, 1H), 6.82 (t, 1H), 7.17-7.42 (m, 6H); LRMS (APCI⁺) 443 [MH⁺].

(4S)-4-benzyl-3-{[(3S,4R)-1-tert-butyl-4-(2,4-difluorophenyl)pyrrolidin-3-yl]carbonyl}-1,3-oxazolidin-2-one-¹H NMR (CDCl₃, 400 MHz) 1.12 (s, 9H), 2.72 (dd, 1H), 2.83 (m, 2H), 3.20 (m, 2H), 3.36 (t, 1H), 4.14 (m, 3H), 4.29 (m, 1H), 4.67 (m, 1H), 6.77 (t, 1H), 6.85 (t, 1H), 7.08 (m, 2H), 7.24 (m, 3H), 7.43 (m, 1H); LRMS (APCI⁺) 443 [MH⁺].

The full relative and absolute stereochemistry of (4S)-4-benzyl-3-{[(3S,4R)-1-tert-butyl-4-(2,4-difluorophenyl)pyrrolidin-3-yl]carbonyl}-1,3-oxazolidin-2-one was determined by X-ray analysis of crystals obtained from ethyl acetate/pentane.

### Preparation 5

### (3S54R)-1-tert-Butyl-4-(2,4-difluorophenyl)pyrrolidine-3-carboxylic acid hydrochloride

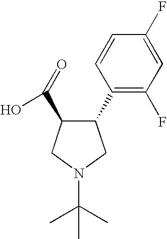

A solution of lithium hydroxide (0.93 g, 39 mmol) in water (15 mL) was added dropwise to a stirred suspension of (4S)-4-benzyl-3-{[(3S,4R)-1-tert-butyl-4-(2,4-difluorophenyl)pyrrolidin-3-yl]carbonyl}-1,3-oxazolidin-2-one (from preparation 4b) (8.63 g, 19.5 mmol) in tetrahydrofuran (50 mL). The resulting reaction mixture was then stirred at room temperature for 1.5 hours, diluted with water (50 mL) and extracted with ethyl acetate (4×150 mL). The aqueous layer was separated, treated with 2M aqueous hydrogen chloride solution (19.5 mL), concentrated to dryness and azeotroped with toluene (5×50 mL). The residual white solid was triturated with dichloromethane (40 mL) and insoluble lithium chloride was removed by filtration. The filtrate was then evaporated to afford the product as a white foam (5.05 g, 92%). ¹H NMR (CD₃OD, 400 MHz) δ 1.44 (s, 9H), 3.36 (m, 2H), 3.64 (t, 1H), 3.25 (dd, 1H), 3.88 (m, 3H), 6.98 (t, 2H), 7.55 (q, 1H); LRMS (APCI⁺) 284 [MH⁺].

### Preparation 6

### (4S)-4-Benzyl-3-{[(3S,4R)-1-benzyl-4-(2,4-difluorophenyl)pyrrolidin-3-yl]carbonyl}-1,3-oxazolidin-2-one

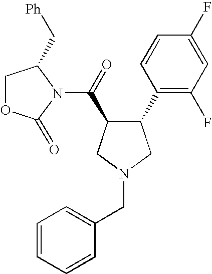

To a stirred solution of (4S)-4-benzyl-3-[(2E)-3-(2,4-difluorophenyl)prop-2-enoyl]-1,3-oxazolidin-2-one (from preparation 3) (46.83 g, 140 mmol) in dichloromethane (300 mL) was added N-methoxymethyl-N-(trimethylsilylmethyl)benzylamine (50.2 mL, 210 mmol) at room temperature. The solution was cooled to −12° C. and a solution of trifluoroacetic acid (1.05 mL) in dichloromethane (10 mL) was added dropwise. The reaction mixture was warmed to room temperature, stirred for 24 hours and saturated sodium hydrogen carbonate solution (180 mL) was added. The phases were separated and the aqueous phase was extracted with dichloromethane (180 mL). The organic extracts were combined, dried over magnesium sulfate, filtered and concentrated in vacuo. Purification of the residue by column chromatography using toluene:methyl tert-butyl ether (12:1) followed by dichloromethane: methyl tert-butyl ether (19:1) as the eluent afforded the title compound (which is the second eluting diastereomer), (63.0 g, 49%). ¹H NMR (CDCl₃, 400 MHz) δ 2.75 (m, 3H), 3.12 (t, 1H), 3.24 (m, 2H), 3.70 (q, 2H) 4.13 (m, 2H), 4.27 (q, 1H), 4.33 (m, 1H), 4.67 (m, 1H), 6.57 (m, 1H), 6.84 (t, 1H), 7.13 (m, 2H), 7.16 (m, 1H), 7.24-7.41 (m, 8H).

### Preparation 7

### Methyl (3S,4R)-1-benzil-4-(2,4-difluorophenyl)pyrrolidine-3-carboxylate

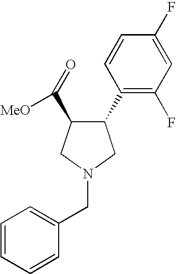

Samarium triflate (6.32 g, 10 mmol) was added to a stirred solution of (4R)-4-benzyl-3-{[(3S,4R)-1-benzyl-4-(2,4-difluorophenyl)pyrrolidin-3-yl]carbonyl}-1,3-oxazolidin-2-one (from preparation 6) (63 g, 130 mmol) in methanol (350 mL) at room temperature. The reaction mixture was stirred for 24 hours and the solvent was removed in vacuo. Dichloromethane (290 mL) was added followed by saturated sodium hydrogen carbonate solution (140 mL) and the mixture was stirred for 15 minutes. The resulting precipitate was filtered and washed with dichloromethane (250 mL) and water (25 mL). The phases were separated and the aqueous layer was extracted with dichloromethane (2×40 mL). The organic extracts were combined, dried over magnesium sulfate, filtered and concentrated in vacuo. The residue was suspended in warm cyclohexane (300 mL) and shaken till formation of a solid occurred. The mixture was allowed to stand at room temperature for 24 hours and the solid was filtered and washed with cold cyclohexane (150 mL). The filtrate was concentrated in vacuo to afford the desired compound, (38 g, 87%). ¹H NMR (CDCl₃, 400 MHz,) δ 2.67 (t, 1H), 2.86 (m, 1H), 2.93 (t, 1H), 3.04 (m, 2H), 3.64 (s, 3H), 3.65 (t, 1H), 3.84 (m, 1H), 6.72 (m, 1H), 6.80 (t, 1H), 7.23 (m, 2H), 7.29-7.38 (m, 5H); [α]²⁵_{D}=−38 (c=0.5, MeOH).

### Preparation 8

### Methyl (3S,4R)-4-(2,4-difluorophenyl)pyrrolidine-3-carboxylate

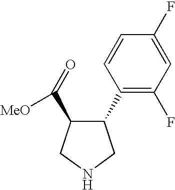

Palladium hydroxide (20% on carbon, 1 g) was added to a solution of methyl (3S,4R)-1-benzyl-4-(2,4-difluorophenyl)pyrrolidine-3-carboxylate (from preparation 7) (10 g, 30 mmol) in ethanol (50 mL) at room temperature. The reaction mixture was hydrogenated at 50 psi for 24 hours and then filtered through Arbocel®, washing with ethanol (50 mL). The solvent was removed in vacuo to give the desired compound as a colourless oil, (7.19 g, 98%). ¹H NMR (CD₃OD, 400 MHz) δ 2.60 (s, 1H), 2.91 (t, 1H), 3.08 (q, 1H), 3.31-3:44 (m, 1H), 3.50 (t, 1H), 3.63 (m, 1H), 3.66 (s, 3H), 6.76 (m, 1H), 6.84 (m, 1H), 7.20 (m, 1H); LRMS (EI⁺) 242 [MH⁺].

### Preparation 9

### Methyl (3S,4R)-1-(6-chloropyridazin-3-yl)-4-(2,4-difluorophenyl)pyrrolidine-3-carboxylate

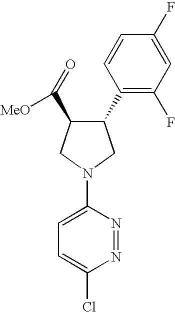

A mixture of methyl (3S,4R)-4-(2,4-difluorophenyl)pyrrolidine-3-carboxylate (from preparation 8) (10.4 g, 43.1 mmol) diisopropylethylamine (75 mL, 430 mmol) and 3,6-dichloropyridazine (22.5 g, 151 mmol) in tetrahydrofuran (90 mL) was heated at reflux for 16 hours. Analysis by tic indicated unreacted amine remaining so a further portion of 3,6-dichloropyridazine (12.0 g, 80.5 mmol) was added and heating was continued for a further 48 hours. After cooling to room temperature the solvent was removed in vacuo and the residue was partitioned between ethyl acetate (400 mL) and water (300 mL). The organic phase was washed with brine (200 mL), dried over magnesium sulfate and concentrated in vacuo. The residue was purified by column chromatography (silica) eluting with ethyl acetate/pentane (1:9 increasing polarity to 4:6) to give the title compound as a yellow oil (11.97 g, 78%). ¹H NMR (CDCl₃, 400 MHz) δ 3.45 (q, 1H), 3.64 (m, 1H), 3.69 (s, 3H), 3.85 (dd, 1H), 3.99-4.10 (m, 3H), 6.66 (d, 1H), 6.81-6.89 (m, 2H), 7.20-7.27 (m, 2H); LRMS (APCI⁺) 354 [MH⁺].

### Preparation 10

### Lithium (3S,4R)-1-(6-chloropyridazin-3-yl)-4-(2,4-difluorophenyl)pyrrolidine-3-carboxylate

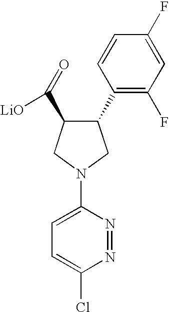

A solution of lithium hydroxide (1.58 g, 65.8 mmol) in water (45 mL) was added dropwise to a solution of methyl (3S,4R)-1-(6-chloropyridazin-3-yl)-4-(2,4-difluorophenyl)pyrrolidine-3-carboxylate (from preparation 9) (21.22 g, 60.0 mmol) in tetrahydrofuran (210 mL) and the mixture was stirred at room temperature for 16 hours. The solvent was removed in vacuo and the residue was azeotroped with toluene (3×80 mL) to give a white solid. This was dissolved in boiling methanol (200 mL) and the solution was allowed to cool to room temperature. Diethyl ether (˜150 mL) was then added gradually to give a white precipitate which was collected by filtration and washed with diethyl ether. Drying in vacuo gave the title compound (11.91 g, 57%). ¹H NMR (CD₃OD, 400 MHz) δ 3.34 (m, 1H), 3.46 (m, 1H), 3.71 (dd, 1H), 3.93-4.10 (m, 3H), 6.88-6.94 (m, 2H), 7.01 (d, 1H), 7.39 (d, 1H), 7.45 (m, 1H); LRMS (APCI⁻) 338 [M-H⁺].

Concentration of the filtrate in vacuo gave a yellow solid which was triturated with boiling ethanol (250 mL). After cooling the ethanol to room temperature diethyl ether (300 mL) was added to precipitate further solid which was collected by filtration and combined with the trituration residue. Drying in vacuo gave 6.81 g (33%) of the title compound.

### Preparation 11

### (3S,4R)-4-(2,4-difluorophenyl)-1-pyridazin-3-ylpyrrolidine-3-carboxylic acid hydrochloride

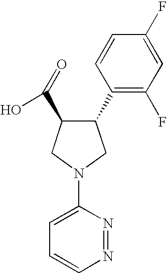

Lithium (3S,4R)-1-(6-chloropyridazin-3-yl)-4-(2,4-difluorophenyl)pyrrolidine-3-carboxylate (from preparation 10) (11.9 g, 34.4 mmol) was suspended in ethanol (110 mL) and 10% palladium on carbon (1.7 g) and 1-methyl-1,4-cyclohexadiene (25 mL, 222 mmol) were added. The mixture was heated at reflux for 2 hours and then a further portion of 1-methyl-1,4-cyclohexadiene (6 mL, 53 mmol) was added. After heating at reflux for a further 2 hours the mixture was cooled and filtered through Arbocel®, washing with ethanol. The filtrate was concentrated in vacuo and azeotroped with toluene (2×50 mL). The residue was triturated with dichloromethane (100 mL) then filtered and dried in vacuo. The yellow solid was taken up in acetone (175 mL) and water (175 mL) with slight heating and then treated with 2M ethereal HCl (50 mL) before being concentrated in vacuo. The residue was taken up in boiling isopropyl alcohol (650 mL), the mixture was filtered, diluted with diisopropyl ether (200 mL) and allowed to cool slowly to room temperature. The resulting precipitate was collected by filtration and washed with diethyl ether. The resulting white solid was boiled in toluene (80 mL) for 15 minutes, the suspension was allowed to cool to room temperature and then concentrated in vacuo. This was then repeated three times to give the title compound as a white solid (6.53 g, 62%). ¹H NMR (CD₃OD, 400 MHz) δ 3.61-3.77 (m, 2H), 3.96 (dd, 1H), 4.08-4.22 (m, 3H), 6.98-7.04 (m, 2H), 7.52 (m, 1H), 7.74 (dd, 1H), 7.89 (dd, 1H), 8.55 (dd, 1H); LRMS (APCI⁺) 306 [MH⁺].

### Preparation 12

### 1-tert-Butyl 3-methyl (3S,4R)-4-(2,4-difluorophenyl)pyrrolidine-1,3-dicarboxylate

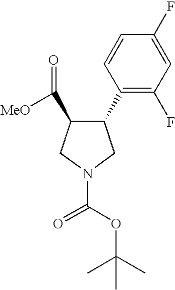

To a solution of methyl (3S,4R)-1-benzyl-4-(2,4-difluorophenyl)pyrrolidine-3-carboxylate, (from preparation 7) (1.0 g, 3.01 mmol), 1-methylcyclohexa-1,4-diene (1.25 mL, 11.12 mmol) and di-tert-butyl dicarbonate (0.72 g, 3.31 mmol) in ethanol (10 mL) was added palladium hydroxide on carbon (0.1 g) at room temperature. The resulting mixture was heated under reflux for 4 hours, cooled to room temperature and filtered through Arbocel®. The filtrate was concentrated in vacuo to give a residue which was partitioned between ethyl acetate (80 mL) and 10% citric acid solution (5 mL). The phases were separated and the organic layer was washed with brine (60 mL), dried over magnesium sulfate, filtered and concentrated in vacuo to give the desired product as a colourless oil (940 mg, 92%). ¹H NMR (CDCl₃, 400 MHz) δ 1.40 (s, 9H), 3.14-3.25 (m, 1H), 3.25-3.40 (m, 1H), 3.48-3.59 (m, 4H), 3.68-3.89 (m, 3H), 6.71-6.82 (m, 2H), 7.15 (m, 1H); LRMS (APCI) 242 [MH⁺—BOC]

### Preparation 13

### (3S,4R)-1-(tert-Butoxycarbonyl)-4-(2,4-difluorophenyl)pyrrolidine-3-carboxylic acid

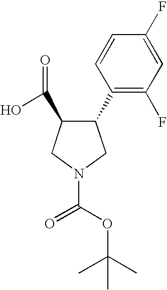

Lithium hydroxide (130 mg, 23.5 mmol) was added dropwise to a stirred solution of 1-tert-butyl 3-methyl (3S,4R)-4-(2,4-difluorophenyl)pyrrolidine-1,3-dicarboxylate (from preparation 12) (930 mg, 2.72 mmol) in tetrahydrofuran (10 mL) at room temperature. The reaction mixture was stirred for 48 hours, concentrated in vacuo and diluted with water (15 mL). The phases were separated and the aqueous phase was extracted with ethyl acetate (25 mL). The aqueous layer was acidified with 2M hydrochloric acid solution (2.7 mL) and further extracted with ethyl acetate (2×40 mL). The combined organic extracts were dried over magnesium sulfate, filtered, concentrated in vacuo and azeotroped with dichloromethane to give the desired product (775 mg, 87%). ¹H NMR (CDCl₃, 400 MHz) δ 1.45 (s, 9H), 3.23-3.46 (m, 2H), 3.56-3.65 (m, 1H), 3.74-3.93 (m, 3H), 6.75-6.87 (m, 2H), 7.20 (m, 1H); LRMS (APCI) 228 [MH⁺—BOC]; LRMS (APCI-)=326 [M−1].

### Preparation 14

### tert-Butyl (3R,4S)-3-(2,4-difluorophenyl)-4-{[3S,4S-3,4-dimethoxy-4-phenylpiperidin-1-yl]carbonyl}pyrrolidine-1-carboxylate

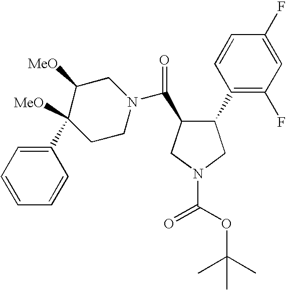

1-Propylphosphonic acid cyclic anhydride (50% in ethyl acetate, 1.6 mL, 2.66 mmol) was added to a mixture of (3S,4S)-3,4-dimethoxy-4-phenylpiperidine (from preparation 21) (589 mg, 2.66 mmol), triethylamine (0.74 mL, 5.32 mmol) and (3S,4R)-1-(tert-butoxycarbonyl)-4-(2,4-difluorophenyl)pyrrolidine-3-carboxylic acid (from preparation 13) (870 mg, 2.66 mmol) in dichloromethane (5 mL) and the mixture was stirred at room temperature for 16 hours. The reaction mixture was diluted with dichloromethane (20 mL) and washed with 10% aqueous potassium carbonate (20 mL) and brine (20 mL), then dried (MgSO₄) and evaporated. The residue was purified by column chromatography (silica) eluting with dichloromethane/methanol/ammonia (99:1:0.1 increasing polarity to 98:2:0.2) to give the title compound as a colourless oil (1.14 g, 81%). ¹H NMR (CDCl₃, 400 MHz) δ 1.42-1.50 (m, 9H), 1.91-2.16 (m, 2H), 2.84-3.18 (m, 7H), 3.29-4.10 (m, 9H), 4.40-4.62 (m, 1H), 6.78-6.91 (m, 2H), 7.21-7.42 (m, 6H); LRMS (APCI⁺) 531 [MH⁺].

### Preparation 15

### (3S,4S)-1-{[(3S,4R)-4-(2,4-Difluorophenyl)pyrrolidin-3-yl]carbonyl}-3,4-dimethoxy-4-phenylpiperidine hydrochloride

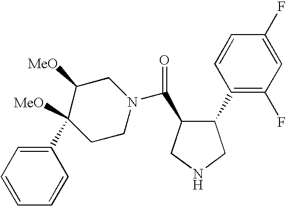

4M HCl in dioxane (10.75 mL) was added to a solution of tert-butyl (3R,4S)-3-(2,4-difluorophenyl)-4-{[(3S,4S)-3,4-dimethoxy-4-phenylpiperidin-1-yl]carbonyl}pyrrolidine-1-carboxylate (from preparation 14) (1.14 g, 2.15 mmol) in dichloromethane (11 mL) and the mixture was stirred at room temperature for 16 hours. The solvent was removed in vacuo and the residue was azeotroped with dichloromethane (30 mL) to give the title compound (859 mg, 86%) which was used without further purification. ¹H NMR (CD₃OD, 400 MHz) δ 1.01-2.42 (m, 2H), 3.00-3.16 (m, 7H), 3.27-3.32 (m, 2H), 3.48-3.98 (m, 7H), 4.22-4.50 (dd, 1H), 7.05-7.18 (m, 2H), 7.22-7.43 (m, 5H), 7.50-7.61 (m, 1H); LRMS (APCI⁺) 431 [MH⁺].

### Preparation 16

### tert-Butyl (3R,4R)-3,4-dihydroxy-4-phenylpiperidine-1-carboxylate

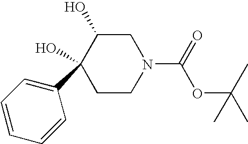

AD-mix β (21.58 g) and methanesulfonamide (1.47 g, 15.4 mmol) were added to water (80 mL) and tert-butanol (80 mL) and the mixture was stirred for 5 minutes at room temperature before being cooled to 0° C. tert-Butyl 4-phenyl-3,6-dihydropyridine-1(2H)-carboxylate (prepared according to Org. Lett. 2001, 3, 2317-2320) (4.0 g, 15.4 mmol) was then added in one portion and the reaction was stirred at 0° C. for 18 hours. Sodium sulfite (13.2 g, 105 mmol) was added and the mixture was stirred at room temperature for 30 minutes before being extracted with ethyl acetate (3×60 mL). The combined organic extracts were washed with 1M NaOH (40 mL), dried (MgSO₄) and evaporated. The residue was purified by column chromatography (silica) eluting with pentane/ethyl acetate (100% pentane increasing polarity to 50% EtOAc in pentane) to give the title compound as an off-white solid (4.18 g, 92%). ¹H NMR (CD₃OD, 400 MHz) δ 1.49 (s, 9H), 1.70 (dt, 1H), 1.90 (td, 1H), 3.00-3.20 (br m, 2H), 3.86-3.91 (m, 2H), 4.02-4.06 (m, 1H), 7.21 (tt, 1H), 7.33 (t, 2H), 7.50 (dd, 2H); LRMS (APCI⁺) 294 [MH⁺]; [α]_{D}^{25=+19.8} (c=0.31, MeOH).

### Preparation 17

### tert-Butyl (3R,4R)-3,4-dimethoxy-4-phenylpiperidine-1-carboxylate

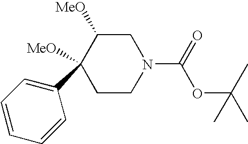

Sodium hydride (87 mg, 2.18 mmol) was added to a solution of tert-butyl (3R,4R)-3,4-dihydroxy-4-phenylpiperidine-1-carboxylate (from preparation 16) (200 mg, 0.68 mmol) in tetrahydrofuran (2 mL) and the mixture was stirred at room temperature for 1 hour. Methyl iodide (144 μL, 2.3 mmol) was then added dropwise over 5 minutes and the mixture was stirred for a further 4 hours. The reaction was cooled to 0° C. and quenched by the addition of water (20 mL). The reaction mixture was extracted with ethyl acetate (2×20 mL) and the combined extracts were washed with brine, dried (MgSO₄) and evaporated to give the title compound as a colourless oil (236 mg) which was used without further purification. ¹H NMR (CDCl₃, 400 MHz) δ1.49 (s, 9H), 1.98-2.12 (m, 2H), 3.11 (s, 3H), 3.16 (s, 3H), 3.12-3.22 (m, 2H), 3.94 (br, 1H), 4.13 (br, 2H), 7.28-7.32 (m, 1H), 7.35-7.39 (m, 2H), 7.42-7.45 (m, 2H); LRMS (APCI⁺) 322 [MH⁺].

### Preparation 18

### (3R,4R)-3,4-Dimethoxy-4-phenylpiperidine hydrochloride

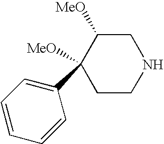

4M HCl in dioxane (4.4 mL) was added to a solution of tert-butyl (3R,4R)-3,4-dimethoxy-4-phenylpiperidine-1-carboxylate (from preparation 17) (230 mg) in dichloromethane (4 mL) and the mixture was stirred at room temperature for 16 hours. The solvent was removed in vacuo and the residue was azeotroped with diethyl ether (3×20 mL) to give the title compound as a white foam (207 mg) which was used without further purification. ¹H NMR (CD₃OD, 400 MHz) δ 2.37 (m, 2H), 3.11 (s, 3H), 3.19 (s, 3H), 3.23 (dd, 1H), 3.25 (dd, 1H), 3.29 (m, 2H), 3.66 (dd, 1H), 7.34-7.38 (m, 1H), 7.41-7.50 (m, 4H); LRMS (APCI⁺) 222 [MH⁺].

### Preparation 19

### tert-Butyl (3S,4S)-3,4-dihydroxy-4-phenylpiperidine-1-carboxylate

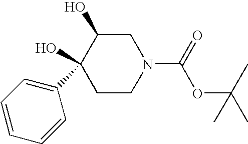

According to the method of preparation 16, but using AD-mix α instead of AD-mix β, tert-butyl 4-phenyl-3,6-dihydropyridine-1(2H)-carboxylate was converted to the title compound. ¹H NMR (CD₃OD, 400 MHz) δ1.49 (s, 9H), 1.70 (dt, 1H), 1.90 (td, 1H), 3.00-3.20 (br m, 2H), 3.86-3.91 (m, 2H), 4.02-4.06 (m, 1H), 7.21 (tt, 1H), 7.33 (t, 2H), 7.50 (dd, 2H); LRMS (APCI⁺) 294 [MH⁺]; [α]_{D}²⁵=−19.4 (c=0.31, MeOH).

### Preparation 20

### tert-Butyl (3S,4S)-3,4-dimethoxy-4-phenylpiperidine-1-carboxylate

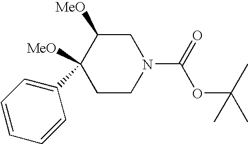

The title compound was formed from the diol of preparation 19 according to the method of preparation 17. ¹H NMR (CDCl₃, 400 MHz) δ 1.49 (s, 9H), 1.98-2.12 (m, 2H), 3.11 (s, 3H), 3.16 (s, 3H), 3.12-3.22 (m, 2H), 3.94 (br, 1H), 4.13 (br, 2H), 7.28-7.32 (m, 1H), 7.35-7.39 (m, 2H), 7.42-7.45 (m, 2H); LRMS (APCI⁺) 322 [MH⁺].

### Preparation 21

### (3S,4S)-3,4-Dimethoxy-4-phenylpiperidine hydrochloride

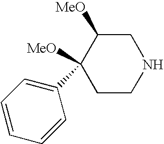

The title compound was formed from the protected piperidine of preparation 20 according to the method of preparation 18. ¹H NMR (CD₃OD, 400 MHz) δ 2.37 (m, 2H), 3.11 (s, 3H), 3.19 (s, 3H), 3.23 (dd, 1H), 3.25 (dd, 1H), 3.29 (m, 2H), 3.66 (dd, 1H), 7.34-7.38 (m, 1H), 7.41-7.50 (m, 4H); LRMS (APCI⁺) 222 [MH⁺].

### Preparation 22

### (3S,4S)-3,4-Dihydroxy-4-phenylpiperidine hydrochloride

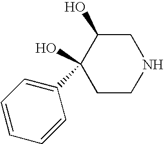

The title compound was formed from the protected piperidine of preparation 19 according to the method of preparation 18. ¹H NMR (CD₃OD, 400 MHz) δ 1.95 (dt, 1H), 2.22 (m, 1H), 3.19-3.38 (m, 4H), 4.21 (dd, 1H), 7.28 (m, 1H), 7.36-7.40 (m, 2H), 7.52-7.56 (m, 2H); LRMS (APCI⁺) 194 [MH⁺].

### Preparation 23

### tert-Butyl (3R,4R)-4-hydroxy-3-methoxy-4-phenylpiperidine-1-carboxylate

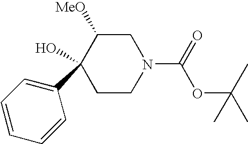

A solution of sodium hydroxide (544 mg, 13.6 mmol) in water (3.4 mL) was added to a solution of tert-butyl (3R,4R)-3,4-dihydroxy-4-phenylpiperidine-1-carboxylate (from preparation 16) (200 mg, 0.68 mmol) in toluene (3.4 mL) followed by methyl iodide (0.85 mL, 13.6 mmol) and tetrabutylammonium hydrogen sulfate (231 mg, 0.68 mmol). The mixture was stirred vigorously at room temperature for 18 hours then diluted with water (20 mL) and extracted with dichloromethane (3×20 mL). The combined organic layers were dried (MgSO₄) and evaporated. The residue was purified by column chromatography (silica) eluting with pentane/ethyl acetate (100% pentane increasing polarity to 30% EtOAc in pentane) to give the title compound as a colourless oil (200 mg, 96%). ¹H NMR (CD₃OD, 400 MHz) δ1.50 (s, 9H), 1.68 (dt, 1H), 1.93 (td 1H), 3.03 (br, 1H), 3.11 (s, 3H), 3.17 (br, 1H), 3.57 (dd, 1H), 3.85-3.90 (m, 1H), 4.19 (br, 1H), 7.23 (tt, 1H), 7.34 (t, 2H), 7.51 (dd, 2H); LRMS (APCI⁺) 208 [MH⁺-Boc].

### Preparation 24

### (3R,4R)-3-methoxy-4-phenylpiperidin-4-ol hydrochloride

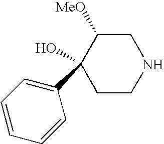

The title compound was formed from the protected piperidine of preparation 23 according to the method of preparation 18. ¹H NMR (CD₃OD, 400 MHz) δ1.93 (dt, 1H), 2.19-2.27 (m, 1H), 3.12 s, 3H, 3.16-3.33 (m, 2H), 3.47 (dd, 1H), 3.88 (dd, 1H), 4.62 (br s, 1H), 7.30 (tt, 1H), 7.40 (t, 2H), 7.54 (d, 2H); LRMS (APCI⁺) 208 [MH⁺].

### Preparation 25

### tert-butyl (3R,4R)-3-ethoxy-4-hydroxy-4-phenylpiperidine-1-carboxylate

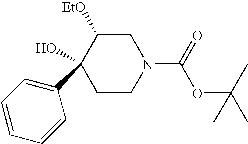

The title compound was formed from the diol of preparation 16 according to the method of preparation 23 using ethyl iodide instead of methyl iodide. ¹H NMR (CD₃OD, 400 MHz) 10.89 (t, 3H), 1.50 (s, 9H), 1.68 (dt, 1H), 1.97 (td, 1H), 3.04-3.22 (m, 3H), 3.36-3.43 (m, 1H), 3.60 (dd, 1H), 3.83-3.92 (m, 1H), 4.09-4.16 (br, 1H), 7.23 (tt, 1H), 7.33 (t, 2H), 7.51 (d, 2H); LRMS (APCI⁺) 222 [MH⁺-Boc].

### Preparation 26

### (3R,4R)-3-ethoxy-4-phenylpiperidin-4-ol hydrochloride

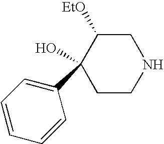

The title compound was formed from the protected piperidine of preparation 25 according to the method of preparation 18. ¹H NMR (CD₃OD, 400 MHz) δ 0.89 (t, 3H), 1.26-1.36 (m, 1H), 1.93 (dt, 1H), 2.23-2.31 (m, 1H), 3.30-3.11 (m, 1H), 3.17-3.45 (m, 4H), 3.92 (dd, 1H), 7.30 (t, 1H), 7.39 (t, 2H), 7.54 (d, 2H); LRMS (APCI⁺) 222 [MH⁺].

### Preparation 27

### tert-Butyl (3S,4S)-4-(4-fluorophenyl)-3,4-dihydroxypiperidine-1-carboxylate

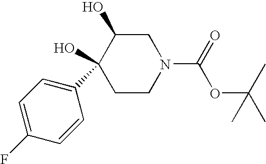

According to the method of preparation 16, but using AD-mix a instead of AD-mix P, tert-butyl 4-(4-fluorophenyl)-3,6-dihydropyridine-1(2H)-carboxylate (prepared according to Synthesis 1991 (11), 993-995) was converted to the title compound. ¹H NMR (CDCl₃, 400 MHz) δ 1.45 (s, 9H), 1.60-1.95 (br, 3H), 2.70 (br, 1H), 2.97 (br, 1H), 3.13 (br, 1H), 3.95 (br, 1H), 4.03 (br, 1H), 4.17 (br, 1H), 7.05 (m, 2H), 7.43 (m, 2H); LRMS (APCI⁺) 312 [MH⁺]; [α]_{D}²⁵=−19.6 (c=0.24, MeOH).

### Preparation 28

### tert-Butyl (3S,4S)-4-(4-fluorophenyl)-3,4-dimethoxypiperidine-1-carboxylate

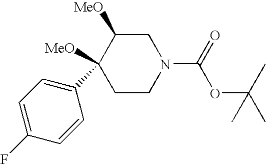

The title compound was formed from the diol of preparation 27 according to the method of preparation 17. ¹H NMR (CDCl₃, 400 MHz) δ 1.46 (s, 9H), 1.93-2.12 (br m, 2H), 2.97-3.22 (br m, 3H), 3.10 (s, 3H), 3.12 (s, 3H), 3.95 (br, 1H), 4.20 (br, 1H), 7.03 (m, 2H), 7.42 (m, 2H); LRMS (APCI⁺) 340 [MH⁺].

### Preparation 29

### (3S,4S)-4-(4-Fluorophenyl)-3,4-dimethoxypiperidine

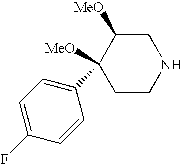

The title compound was formed from the protected piperidine of preparation 28 according to the method of preparation 18. ¹H NMR (CD₃OD, 400 MHz) δ 2.37 (m, 2H), 3.10 (s, 3H), 3.20 (s, 3H), 3.20-3.38 (m, 4H), 3.62 (m, 1H), 7.18 (m, 2H), 7.50 (m, 2H); LRMS (APCI⁺) 240 [MH⁺]; [α]_{D}²⁵+24.5 (c=0.21, MeOH).

### Preparation 30

### tert-Butyl (3R,4R)-4-(4-fluorophenyl)-3,4-dihydroxypiperidine-1-carboxylate

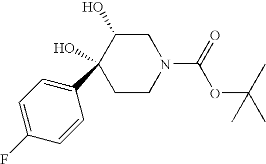

According to the method of preparation 16, tert-butyl 4-(4-fluorophenyl)-3,6-dihydropyridine-1(2H)-carboxylate (prepared according to Synthesis 1991 (11), 993-995) was converted to the title compound. ¹H NMR (CDCl₃, 400 MHz). 1.45 (s, 9H), 1.65 (br, 1H), 1.82 (br, 2H), 2.68 (br, 1H), 2.97 (br, 1H), 3.13 (br, 1H), 3.95 (br, 1H), 4.03 (br, 1H), 4.17 (br, 1H), 7.05 (m, 2H), 7.43 (m, 2H); LRMS (APCI⁺) 312 [MH⁺]; [α]_{D}^{25=+25.7} (c=0.23, MeOH).

### Preparation 31

### tert-Butyl (3R,4R)-4-(4-fluorophenyl)-3,4-dimethoxypiperidine-1-carboxylate

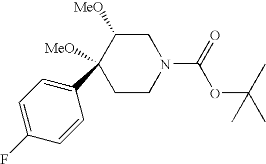

The title compound was formed from the diol of preparation 30 according to the method of preparation 17. ¹H NMR (CDCl₃, 400 MHz) δ 1.46 (s, 9H), 1.93-2.12 (br m, 2H), 2.97-3.22 (br m, 3H), 3.10 (s, 3H), 3.12 (s, 3H), 3.95 (br, 1H), 4.20 (br, 1H), 7.03 (m, 2H), 7.42 (m, 2H); LRMS (APCI⁺) 340 [MH⁺].

### Preparation 32

### (3R,4R)-4-(4-Fluorophenyl)-3,4-dimethoxypiperidine

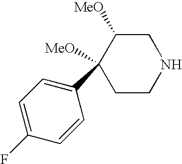

The title compound was formed from the protected piperidine of preparation 31 according to the method of preparation 18. ¹H NMR (CD₃OD, 400 MHz) δ 2.37 (m, 2H), 3.10 (s, 3H), 3.20 (s, 3H), 3.20-3.38 (m, 4H), 3.62 (m, 1H), 7.18 (m, 2H), 7.50 (m, 2H); LRMS (APCI⁺) 240 [MH⁺]; [α]D25=−20.7 (c=0.19, MeOH).

### Preparation 33

### tert-Butyl (3S,4S)-4-(4-fluorophenyl)-4-hydroxy-3-methoxypiperidine-1-carboxylate

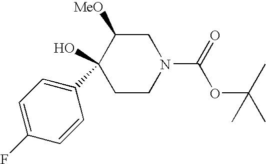

The title compound was formed from the diol of preparation 27 according to the method of preparation 23. ¹H NMR (CD₃OD, 400 MHz) δ 1.45 (s, 9H), 1.65 (m, 1H), 1.90 (m, 1H), 3.00 (br, 1H), 3.10 (s, 3H), 3.08-3.22 (br m, 1H), 3.53 (m, 1H), 3.87 (m, 1H), 4.20 (br, 1H), 7.03 (m, 2H), 7.52 (m, 2H); LRMS (APCI⁺) 326 [MH⁺].

### Preparation 34

### (3R,4R)-4-(4-Fluorophenyl)-3,4-dimethoxypiperidine

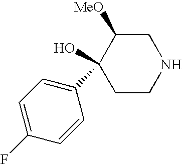

The title compound was formed from the protected piperidine of preparation 33 according to the method of preparation 18. ¹H NMR (CD₃OD, 400 MHz) δ 1.95 (m, 1H), 2.20 (m, 1H), 3.13 (s, 3H), 3.15-3.40 (m, 3H), 3.48 (m, 1H), 3.81 (m, 1H), 7.10 (m, 2H), 7.55 (m, 2H); LRMS (APCI⁺) 226 [MH⁺].

### Preparation 35

### tert-Butyl (3S,4S)-3-(benzyloxy)-4-hydroxy-4-phenylpiperidine-1-carboxylate

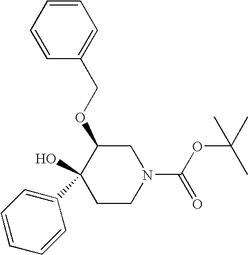

A solution of sodium hydroxide (816 mg, 20.4 mmol) in water (10.2 mL) was added to a solution of tert-butyl (3S,4S)-3,4-dihydroxy-4-phenylpiperidine-1-carboxylate (from preparation 19) (600 mg, 2.04 mmol) in toluene (10.2 mL). Tetrabutylammonium sulfate (346 mg, 1.02 mmol) and benzyl bromide (1.05 g, 6.12 mmol) were added and the reaction mixture was stirred vigorously at room temperature overnight. The resulting mixture was diluted with water (30 mL) and extracted with dichloromethane (3×100 mL). The combined organic solution was dried over magnesium sulfate, concentrated in vacuo and purified by column chromatography (silica) eluting with pentane:ethyl acetate (1:0 to 7:3) to afford the title compound as a colourless oil (760 g, 97%). ¹H NMR (CD₃OD, 400 MHz) δ 1.47 (s, 9H), 1.69 (dt, 1H), 1.96 (td, 1H), 3.05-3.25 (m, br, 2H), 3.65-3.75 (m, br, 1H), 3.86-3.91 (m, 1H), 4.00-4.30 (m, br, 3H), 6.97 (d, br, 2H), 7.19 (s, br, 3H), 7.26 (t, 1H), 7.35 (t, 2H), 7.48 (d, 2H); LRMS (APCI⁺) 401 [MNa⁺], 384 [MH⁺].

### Preparation 36

### tert-Butyl (3S,4S)-3-(benzyloxy)-4-methoxy-4-phenylpiperidine-1-carboxylate

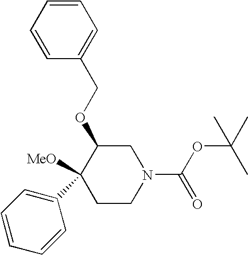

To a stirred solution of the product of preparation 35 (369 mg, 0.96 mmol) and potassium hydroxide (215 mg, 3.84 mmol) in dimethylsulfoxide (5 mL) was added methyl iodide (0.12 mL, 1.92 mmol) and the reaction mixture was stirred at room temperature overnight. The precipitate formed was dissolved with dimethylsulfoxide (3 mL) and the reaction mixture was stirred for another 2 days before being poured into brine (30 mL). The aqueous layer was extracted with diethyl ether (3×50 mL), the combined organic solution was dried over magnesium sulfate and concentrated in vacuo. The residue was purified by column chromatography (silica) eluting with pentane:ethyl acetate (1:0 to 8:2) to afford the title compound as a white solid (374 mg, 98%). ¹H NMR (CD₃OD, 400 MHz) δ 1.45 (s, 9H), 2.07-2.15 (m, 2H), 3.00-3.27 (m, 5H), 3.37-3.48 (m, 1H), 3.86 (dt, 1H), 4.10-4.20 (m, 3H), 7.01-7.04 (m, 2H), 7.20-7.22 (m, 3H), 7.33 (t, 1H), 7.39 (t, 2H), 7.45 (d, 2H); LRMS (APCI⁺) 398 [MH⁺].

### Preparation 37

### tert-Butyl (3S,4S)-3-hydroxy-4-methoxy-4-phenylpiperidine-1-carboxylate

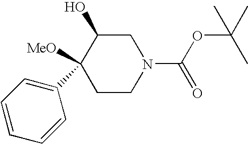

To a solution of tert-butyl (3S,4S)-3-(benzyloxy)-4-methoxy-4-phenylpiperidine-1-carboxylate (from preparation 36) (335 mg, 0.84 mmol) in ethanol (5 mL) was added 20% palladium on carbon (50 mg, catalytic amount) and 1-methyl-1,4-cyclohexadiene (0.28 mL, 2.52 mmol). The reaction mixture was heated at reflux for 1.5 h, filtered through Arbocel®) and washed with ethanol (100 mL). The filtrate was concentrated in vacuo and the residue was purified by column chromatography (silica) eluting with pentane:ethyl acetate (1:0 to 7:3) to afford the title compound as a white foam (258 mg, 100%). ¹H NMR (CD₃OD, 400 MHz) δ 1.48 (s, 9H), 2.11-2.19 (m, 2H), 3.00-3.25 (m, 5H), 3.49 (dd, 1H), 3.89-3.93 (m, 2H), 7.27 (t, 1H), 7.37 (t, 2H), 7.46 (d, 2H); LRMS (APCI⁺) 325 [MNH₄⁺], 308 [MH⁺].

### Preparation 38

### (3S,4S)-4-Methoxy-4-phenylpiperidin-3-ol

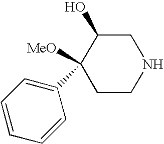

4M HCl in dioxane (2.0 mL) was added to a solution of tert-butyl (3S,4S)-3-hydroxy-4-methoxy-4-phenylpiperidine-1-carboxylate (from preparation 37) (248 mg) in dichloromethane (2 mL). The product crystallised on standing for 1 h, the solid was washed with diethyl ether (20 mL) and pentane (5 mL) and dried under vacuum to give the title compound as a white solid (184 mg, 93%). ¹H NMR (CD₃OD, 400 MHz) δ 2.38-2.48 (m, 2H), 3.12 (s, 3H), 3.15 (dd, 1H), 3.22-3.28 (m, 2H), 3.29-3.33 (m, 1H), 3.94 (dd, 1H), 7.35 (t, 1H), 7.43 (t, 2H), 7.49 (d, 2H); LRMS (APCI⁺) 208 [MH⁺].

### Preparation 39

### (3R,4R)-4-Methoxy-4-phenylpiperidin-3-ol

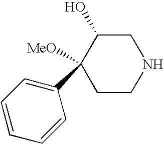

The title compound was formed from the product of preparation 16 according to the methods of preparations 35-38. LRMS (APCI⁺) 207 [MH⁺].

### Preparation 40

### (3S,4S)-4-Ethoxy-4-phenylpiperidin-3-ol

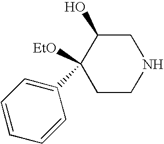

The title compound was formed from the product of preparation 19 using ethyl iodide instead of methyl iodide according to the methods of preparations 35-38. LRMS (APCI⁺) 221 [MH⁺].

### Preparation 41

### (3S,4S)-3-Methoxy-4-phenylpiperidin-4-ol

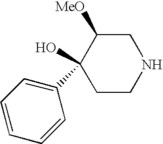

The title compound was formed from the diol of preparation 19 according to the method of preparation 23 followed by subsequent deprotection as described in preparation 18. LRMS (APCI⁺) 207 [MH⁺].

### Preparation 42

### (3S,4S)-3-Ethoxy-4-Phenylpiperidin-4-ol

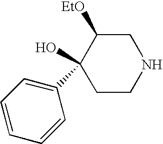

The title compound was formed from the diol of preparation 19 according to the method of preparation 23 using ethyl iodide instead of methyl iodide, followed by subsequent deprotection as described in preparation 18. LRMS (APCI⁺) 221 [MH⁺].

### Preparation 43

### 1-Benzyl-3,3-dimethylpiperidin-4-one

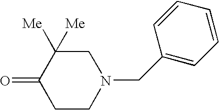

To a solution of formaldehyde (16.9 mL, 0.23 mmol) in ethanol (50 mL) cooled to 10° C. was added benzylamine (10.9 mL, 0.10 mmol) dropwise over 1 h. The resulting solution was added over 2 h to a refluxing solution of 3-methylbutane-2-one in ethanol (50 mL) followed by concentrated hydrochloric acid (9.2 mL). The reaction mixture was heated at reflux overnight, cooled to room temperature before N,N-diisopropylethylamine (19.2 mL, 0.11 mmol) and formaldehyde (2.3 mL, 0.03 mmol) were added. The mixture was heated again at reflux for 6 h, cooled to room temperature and treated with a solution of potassium hydroxide (6.2 g) in water (20 mL). The precipitate formed was dissolved in water (50 mL) and the resulting solution was extracted with ethyl acetate (3×100 mL). The combined organic phase was washed with brine, concentrated in vacuo and the residue was purified by column chromatography (Silica) eluting with dichloromethane:pentane (1:1 to 1:0) to afford the title compound as an oil (9.63 g, 44%). ¹H NMR (CDCl₃, 400 MHz) δ 1.11 (s, 6H), 2.38 (s, 2H), 2.49 (t, 2H), 2.70 (t, 2H), 3.54 (s, 2H), 7.21-7.37 (m, 5H); LRMS 218 [MH⁺].

### Preparation 44

### 1-Benzyl-3,3-dimethyl-4-phenylpiperidin-4-ol

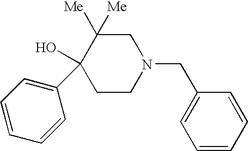

To a solution of anhydrous diethyl ether (50 mL) cooled at −70° C. was added a 2M phenyl lithium solution in diethyl ether (5.6 mL, 8.6 mmol) and the reaction mixture was stirred at −70° C. while a solution of the product from preparation 43 (2.20 g, 10.1 mmol) in diethyl ether (20 mL) was added over 30 min. After addition was complete the reaction mixture was allowed to warm up to 10° C. before being treated with a saturated ammonium chloride solution and diluted with water. The organic layer was separated, washed with water and brine, dried over sodium sulfate and concentrated in vacuo. The residue was purified by column chromatography eluting with dichloromethane:pentane (99:1 to 90:10) to afford the title compound (2.18 g, 73%). ¹H NMR (CDCl₃, 400 MHz) δ 0.75 (s, 3H), 0.96 (s, 3H), 1.40-1.59 (m, 2H), 2.30 (d, 1H), 2.41 (d, 1H), 2.52 (m, 1H), 2.78-2.93 (m, 2H), 3.49 (d, 1H), 3.59 (d, 1H), 7.21-7.41 (m, 8H), 7.47-7.52 (m, 2H); LRMS (APCI⁺) 296 [MH⁺], (ESI⁺) 296 [MH⁺].

### Preparation 45a (4S)-3,3-Dimethyl-4-phenylpiperidin-4-ol and

### Preparation 45b (4R)-3,3-Dimethyl-4-phenylpiperidin-4-ol

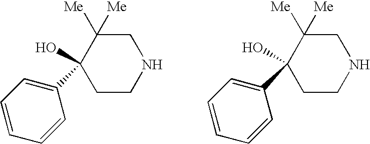

To a solution of the product of preparation 44 (2.10 g, 7.10 mmol) in ethanol (25 mL) was added 20% palladium hydroxide on carbon (100 mg, catalytic amount), 1-methyl-1,4-cyclohexadiene (3.5 mL, 31.2 mmol) and the reaction was heated to 75° C. for 2 h. The mixture was cooled to room temperature overnight, filtered through Arbocel® and washed with ethanol. The filtrate was concentrated in vacuo and the residual solid was purified by trituration in pentane to afford the racemic mixture as a pale pink solid (1.35 g, 93%).

The enantiomers were separated by chiral phase HPLC eluting with isopropyl alcohol: hexane:diethylamine (30:70:0.1) to afford (4S)-3,3-dimethyl-4-phenylpiperidin-4-ol (Prep 45a) and (4R)-3,3-dimethyl-4-phenylpiperidin-4-ol (Prep 45b). ¹H NMR (CDCl₃, 400 MHz) δ 0.78 (s, 3H), 0.89 (s, 3H), 1.40 (d, 1H), 1.91 (b, 2H), 2.45 (d, 1H), 2.66 (m, 1H), 3.02 (m, 1H), 3.10-3.24 (m, 2H), 7.22-7.29 (m, 1H), 7.30-7.37 (m, 2H), 7.45-7.51 (m, 2H); LRMS (APCI⁺) 206 [MH⁺], (ESI⁺) 206 [MH⁺].

### Preparation 46

### 1-tert-Butyl-4-ethyl-3-oxopiperidine-1,4-dicarboxylate

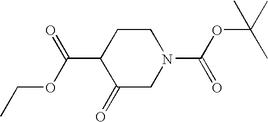

To a solution of ethyl-1-benzyl-3-oxopiperidine-4-carboxylate hydrochloride (51.3 g, 170 mmol) in ethanol (200 mL) and water (200 mL) was added di-tert-butyl dicarbonate (40.8 g, 187 mmol), sodium hydrogen carbonate (14.3 g, 170 mmol), palladium on carbon (18.1 g, 17.0 mmol) and the reaction mixture was hydrogenated at room temperature at 10 Bar for 48 h. The mixture was filtered through Arbocel® and the filtrate was concentrated in vacuo. The residue was partitioned between ethyl acetate and water, the aqueous phase was extracted with ethyl acetate and the combined organic solution was evaporated to give the title compound as a brown oil (44.1 g, 83%). ¹H NMR (300 MHz, CDCl₃) δ 12.03 (s, 1H), 4.22 (q, 2H), 4.05 (s, br, 2H), 3.49-3.50 (m, 2H), 2.35-2.36 (m, 2H), 1.46 (s, 9H), 1.30 (t, 3H); LC-MS (ESI⁻): 270 [M-H].

### Preparation 47

### 1-tert-Butyl-4-ethyl-5-{[(4-methylphenyl)sulfonyl]oxy}-3e-4-dihydropiperidine-1,4(2H)-dicarboxylate

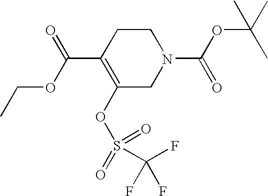

A solution of 1-tert-butyl-4-ethyl-3-oxopiperidine-1,4-dicarboxylate (from preparation 46) (37.9 g, 140 mmol) and N,N-diisopropylethylamine (27.7 mL, 168 mmol) in dichloromethane (900 mL) was cooled to −78° C. under a nitrogen atmosphere. Trifluoromethanesulfonic anhydride was added dropwise, keeping the temperature below −70° C. and the reaction mixture was stirred at this temperature for 45 min then at room temperature for 3 h. The reaction mixture was treated with a saturated ammonium chloride solution, the organic phase was decanted and the aqueous phase was extracted with dichloromethane.

The combined organic solution was dried over sodium sulfate and concentrated in vacuo to afford the title compound as a red solid (67.2 g), which was taken onto the next step without further purification. ¹H NMR (300 MHz, CDCl₃) δ 4.41 (q, 2H), 4.09 (s, br, 2H), 3.49-3.51 (m, 2H), 2.60-2.61 (m, 2H), 1.49 (s, 9H), 1.32 (t, 3H); LC-MS (ESI⁻) 537 [2×M-H]. Preparation 48

### 1-tert-Butyl-4-ethyl-5-(2,4-difluorophenyl)-3,4-dihydropiperidine-1,4(2H)-dicarboxylate

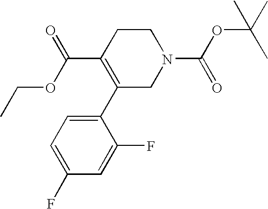

A solution of 1-tert-butyl-4-ethyl-5-{[(4-methylphenyl)sulfonyl}oxy}-3,4-dihydropiperidine-1,4(2H)-dicarboxylate (from preparation 47) (67.2 g, 167 mmol) in toluene (1 L) and ethanol (500 mL) was degassed by bubbling nitrogen through the solution for 45 min. 2,4-Difluorophenyl boronic acid (28.9 g, 183 mmol) and dichloro[1,1′-bis(diphenylphosphino)ferrocene]palladium (0.63 g, catalytic amount) were added and the reaction mixture was heated to 80° C. before a 2M sodium carbonate solution (45.9 g, 433 mmol) was added. The resulting mixture was refluxed overnight, cooled to room temperature and filtered through Arbocel®. The organic layer was decanted, the aqueous was extracted with ethyl acetate and the combined organic solution was washed with brine and concentrated in vacuo. The residue was purified by column chromatography (silica) eluting with ethyl acetate:hexane (1:2) to afford the title compound as a yellow oil (19.9 g, 33%). ¹H NMR (300 MHz, CDCl₃) δ 7.06-7.18 (m, 1H), 6.71-6.86 (m, 2H), 4.10 (s, br, 2H), 3.95 (q, 2H), 3.55-3.61 (m, 2H), 2.56 (s, br, 2H), 147 (s, 9H), 0.97 (t, 3H); LC-MS (ESI⁺) 268 [MH⁺].

### Preparation 49

### 1-tert-Butyl-4-ethyl-3-(2,4-difluorophenyl)piperidine-1,4-dicarboxylate

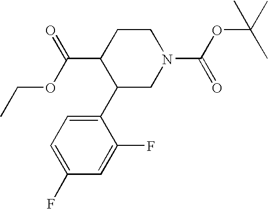

To a solution of 1-tert-butyl-4-ethyl-5-(2,4-difluorophenyl)-3,4-dihydropiperidine-1,4(2H)-dicarboxylate (from preparation 48) (19.9 g, 54.2 mmol) in methanol (750 mL) was added crunched magnesium (13.2 g, 543 mmol) portionwise and the reaction mixture was stirred at room temperature overnight. The resulting mixture was treated with 1M HCl (650 mL) and extracted with ethyl acetate. The organic solution was dried over sodium sulfate and concentrated in vacuo to afford the title compound as a brown oil (18.1 g, 91%). ¹H NMR (300 MHz, CDCl₃) δ 7.14-7.18 (m, 1H), 6.73-6.79 (t, 2H), 3.89-4.03 (m, 2H), 3.56-3.72 (m, 2H), 3.46-3.49 (m, 2H), 3.37-3.38 (m, 1H), 3.01-3.04 (m, 1H), 1.87-2.07 (m, 2H), 1.49 (s, 9H), 1.02 (t, 3H); GC-MS (EI) 369 [M⁺].

### Preparation 50

### 1-(tert-Butoxycarbonyl)-3-(2,4-difluorophenyl)piperidine-4-carboxylic acid

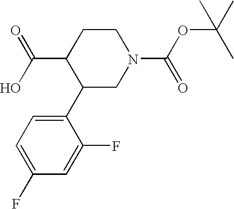

To a solution of 1-tert-butyl-4-ethyl-3-(2,4-difluorophenyl)piperidine-1,4-dicarboxylate (from preparation

49) (155 mg, 0.42 mmol) in methanol (5 mL) was added sodium (50.0 mg, 2.10 mmol) portionwise and the reaction mixture was heated to 75° C. for 1 h. A 5M sodium hydroxide solution (0.5 mL) was added and the resulting mixture was heated to 100° C. for 45 min. The reaction mixture was cooled to room temperature, acidified to pH-5 with 2M HCl and extracted with ethyl acetate. The organic solution was dried over sodium sulfate and concentrated in vacuo to afford the title compound (59 mg, 41%). ¹H NMR (300 MHz, CDCl₃) δ 7.08-7.16 (m, 1H), 6.70-6.79 (m, 2H), 4.11-4.19 (m, 1H), 3.07-3.14 (m, 1H), 2.73-2.89 (m, 2H), 1.98-2.03 (m, 1H), 1.65-1.74 (m, 1H), 1.38-0.89 (m, 12H); LC-MS (ESI⁻) 340 [M-H].

### Preparation 51a tert-Butyl-(3R,4R)-4-{[(4S)-4-benzyl-2-oxo-1,3-oxazolidine-3-yl]carbonyl-3-(2,4-difluorophenyl)piperidine-1-carboxylate and

### Preparation 51b

### tert-Butyl-(3S,4S)-4-{[(4S)-4-benzyl-2-oxo-1,3-oxazolidine-3-yl]carbonyl}-3-(2,4-difluorophenyl)piperidine-1-carboxylate

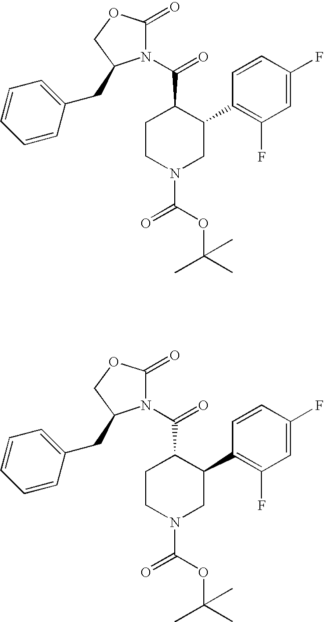

To a solution of 1-tert-butoxycarbonyl)-3-(2,4-difluorophenyl)piperidine-4-carboxylic acid (from preparation 50) (41.0 g, 120 mmol) and triethylamine (41.9 mL, 300 mmol) in freshly distilled tetrahydrofuran (800 mL) cooled to −20° C., was added pivaloyl chloride (18.8 g, 156 mmol) and the reaction mixture was stirred at −20° C. for 1 h. Lithium chloride (8.15 mg, 192 mmol) and (S)-(−)-4-benzyl-2-oxazolidinone (27.7 mg, 156 mmol) were added and the resulting mixture was stirred at room temperature overnight. The solution was treated with 0.5M citric acid solution, extracted with dichloromethane and the organic phase was concentrated in vacuo to give the crude mixture of diastereoisomers. Separation was achieved by column chromatography (silica) eluting with ethyl acetate:hexane (1:3) to afford 7.3 g of tert-butyl-(3S,4S)-4-{[(4S)-4-benzyl-2-oxo-1,3-oxazolidine-3-yl}carbonyl}-3-(2,4-difluorophenyl)piperidine-1-carboxylate (Prep 51b) and 15.6 g of the desired tert-butyl-(3R,4R)-4-{[(4S)-4-benzyl-2-oxo-1,3-oxazolidine-3-yl}carbonyl}-3-(2,4-difluorophenyl)piperidine-1-carboxylate (Prep 51a). ¹H NMR (400 MHz, CDCl₃) δ 7.35-6.70-7.35 (m, 8H), 4.48-4.60 (m, 1H), 4.34-4.45 (m, 1H), 4.18-4.32 (m, 1H), 4.08-4.16 (m, 1H), 4.00-4.06 (m, 1H), 3.37-3.55 (m, 1H), 2.70-2.95 (m, 3H), 2.20-2.35 (m, 1H), 1.95-2.05 (m, 1H), 1.62-1.80 (m, 1H), 1.40-1.60 (m, 10H); LC-MS (ESI⁺) 445 [MH⁺-tert-butyl].

### Preparation 52

### 1-tert-Butyl-4-methyl-(3R,4R)-3-(2,4-difluorophenyl)piperidine-1,4-dicarboxylate

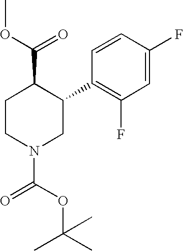

A mixture of tert-butyl-(3R,4R)-4-{[(4S)-4-benzyl-2-oxo-1,3-oxazolidine-3-yl}carbonyl}-3-(2,4-difluorophenyl)piperidine-1-carboxylate (from preparation 51a) (4.90 g, 9.8 mmol), dimethyl carbonate (4.40 g, 49.0 mmol) and sodium methoxide (2.60 g, 49.0 mmol) in dichloromethane (20 mL) was stirred at room temperature for 16 h under a nitrogen atmosphere. The reaction mixture was quenched with water, extracted with dichloromethane and the organic solution was concentrated in vacuo. The residue was purified by column chromatography (silica) eluting with ethyl acetate:hexane (1:2) to afford the title compound as a colourless oil (3.1 g, 89%); LC-MS (ESI⁺) 356 (MH+).

### Preparation 53

### (3R,4R)-1-(tert-Butoxycarbonyl)-3-(2,4-difluorophenyl)piperidine-4-carboxylic acid

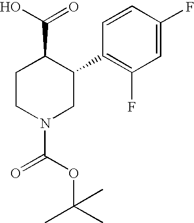

A solution of 1-tert-butyl-4-methyl-(3R,4R)-3-(2,4-difluorophenyl)piperidine-1,4-dicarboxylate (from preparation 52) (3.10 g, 8.70 mmol) and lithium hydroxide (0.44 g, 10.5 mmol) in water (3 mL) and tetrahydrofuran (8 mL) was stirred at room temperature over the weekend. The solvent was evaporated in vacuo and the residue was acidified with 1M HCl. The precipitate formed was filtered, washed with water and dried under vacuum to afford the title compound as a white solid (1.47 g). The acidic layer was extracted with dichloromethane (3×100 mL) and the combined organic solution was concentrated to give another 1.46 g of the desired product as a white solid (total yield 93%). ¹H NMR (300 MHz, CDCl₃) δ 7.08-7.16 (m, 1H), 6.70-6.79 (m, 2H), 4.11-4.19 (m, 1H), 3.07-3.14 (m, 1H), 2.73-2.89 (m, 2H), 1.98-2.03 (m, 1H), 1.65-1.74 (m, 1H), 1.38-0.89 (m, 12H); LC-MS (ESI⁻) 340 [M-H].

### Preparation 54

### Methyl (3S,4R)-4-(2,4-difluorophenyl)-1-(5-fluoropyridine-3-yl)pyrrolidine-3-carboxylate

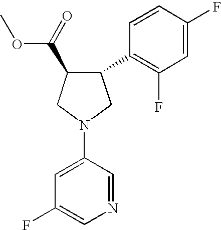

To a solution of methyl (3S,4R)-4-(2,4-difluorophenyl)pyrrolidine-3-carboxylate (from preparation 8) (208 mg, 0.72 mmol) in toluene (10 mL) was added sodium tent-butoxide (134 mg, 1.80 mmol), 3-bromo-5-fluoropyridine (445 mg, 2.52 mmol), 2,2′-bis(diphenylphosphino)-1,1′-binaphthyl (90.0 mg, 0.14 mmol) and palladium dibenzylideneacetone (37.0 mg, 0.07 mmol) and the reaction mixture was heated to 100° C. for 1 h under a nitrogen atmosphere. The mixture was filtered, washed with toluene and the solvent was evaporated. The residual oil was purified by column chromatography (silica) eluting with dichloromethane:methanol (99:1 to 98:2) to afford the title compound as an orange oil (127 mg, 52%). ¹H NMR (CDCl₃, 400 MHz) δ 3.46 (m, 2H), 3.65 (m, 1H), 3.69 (s, 3H), 3.79 (m, 2H), 4.02 (q, 1H), 6.86 (m, 2H), 7.19 (m, 1H), 7.81 (s, 1H), 7.86 (s, 1H); LRMS (APCI⁺) 337 [MH⁺].

### Preparation 55

### (3S,4R)-4-(2,4-Difluorophenyl)-1-(5-fluoropyridine-3-yl)pyrrolidine-3-carboxylic acid

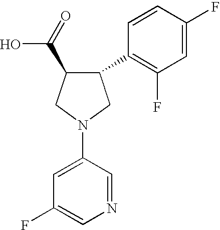

A solution of the product from preparation 54 (340 mg, 1 mmol) and 1M sodium hydroxide (2 mL, 2 mmol) in methanol (2 mL) was stirred at room temperature overnight. The solvent was removed and the residue was partitioned between water and ethyl acetate. The aqueous layer was acidified with 2M hydrochloric acid solution (1 mL) and the organic solution was decanted, dried over magnesium sulfate and concentrated in vacuo. The residue was purified by column chromatography (silica) eluting with dichloromethane:methanol (98:2 to 95:5) to afford the title compound as a yellow solid (99 mg, 30%). ¹H NMR (CD₃OD, 400 MHz) δ 3.44 (m, 2H), 3.65 (t, 1H), 3.81 (m, 2H), 4.02 (m, 1H), 6.81 (d, 1H), 6.94 (m, 2H), 7.40 (m, 2H), 7.75 (br d, 2H); LRMS (APCI⁺) 323 [MH⁺].

### Preparation 56

### Methyl (3S,4R)-1-(5-cyanopyridin-2-yl)-4-(2,4-difluorophenyl)pyrrolidine-3-carboxylate

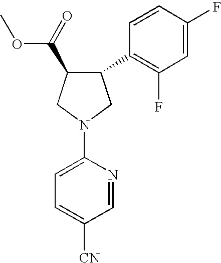

To a solution of methyl (3S,4R)-4-(2,4-difluorophenyl)pyrrolidine-3-carboxylate (from preparation 8) (1.5 g, 5.40 mmol) in acetonitrile (15 mL) was added N,N-diisopropylethylamine (3.76 mL, 21.0 mmol), 2-chloro-5-cyanopyridine (1.12 g, 8.10 mmol) and the reaction mixture was heated at 70° C. under a nitrogen atmosphere. The solvent was removed, the residue was taken up in ethyl acetate and the white precipitate formed was filtered to give 778 mg of product. The organic solution was washed with water (50 mL) and brine (50 mL), dried over magnesium sulfate, concentrated in vacuo to give an orange solid. The orange solid was purified by trituration in methanol and filtration to afford the title compound as a white solid (935 mg). Both crops were combined to give an overall yield of 92%. ¹H NMR (CD₃OD, 400 MHz) δ 3.56 (m, 2H), 3.66 (s, 3H), 3.80 (t, 1H), 4.02 (m, 3H), 6.61 (d, 1H), 6.98 (m, 2H), 7.44 (m, 1H), 7.74 (d, 2H), 8.40 (s, 1H); LRMS (APCI⁺) 344 [MH⁺].

### Preparation 57

### (3S,4R)-1-(5-Cyanopyridin-2-yl)-4-(2,4-difluorophenyl)pyrrolidine-3-carboxylic acid

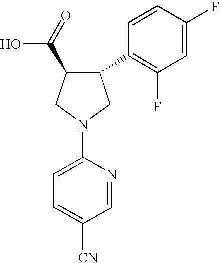

To a solution of the product of preparation 56 (1.71 g, 4.98 mmol) in dioxane (20 mL) was added a solution of sodium hydroxide (598 mg, 14.9 mmol) in water (10 mL) and the mixture was stirred at room temperature overnight. The solvent was evaporated, the residue was dissolved in water (30 mL) and washed with ether (30 mL). The aqueous layer was acidified with 2M hydrochloric acid solution to pH=3 (7.45 mL) and concentrated in vacuo. The residue was stirred with acetonitrile for 30 min, inorganics were filtered off and the solution was acidified further with 2M hydrochloric acid. The solvent was removed to afford the title compound as a white solid (1.3 g, 72%). ¹H NMR (CD₃OD, 400 MHz) δ 3.61 (m, 1H), 3.69 (t, 1H), 3.93 (t, 1H), 4.12 (m, 3H), 6.94 (m, 2H), 6.98 (m, 2H), 7.06 (d, 1H), 7.45 (m, 1H), 8.02 (d, 1H), 8.5 (s, 1H); LRMS (APCI⁺) 330 [MH⁺].

### Preparation 58

### 3-Methyl-4-phenyl-1,2,3,6-tetrahydropyridine

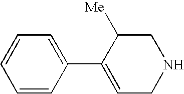

1-Chloroethyl chloroformate (10.78 mL, 99 mmol) was added dropwise to a solution of 1-benzyl-3-methyl-4-phenyl-1,2,3,6-tetrahydropyridine (Prepared according to Tetrahedron Lett. 1965, 21, 3387) (6.57 g, 25 mmol) in dichloromethane (50 mL) and N,N-diisopropylethylamine (9.57 mL, 55 mmol) at 0° C. The mixture was heated at reflux for 3 h, cooled and the solvent was removed in vacuo. The residue was taken up dichloromethane (50 mL), washed with 10% aqueous citric acid (50 mL) and water (50 mL), dried (MgSO₄) and evaporated. The residue was taken up in methanol (50 mL), heated at reflux for 3 h then cooled and evaporated. The reside was purified by column chromatography (silica) eluting with dichloromethane/methanol/ammonia (95:5:0.5) to give the title compound as a colourless oil (3.06 g, 70%). ¹H NMR (CD₃OD, 400 MHz) δ 1.01 (d, 3H), 3.16 (m, 2H), 3.49 (m, 1H), 3.76 (d, 2H), 5.85 (t, 1H), 7.20-7.30 (m, 5H); LRMS (APCI+) 174 [MH+].

### Preparation 59

### Tert-butyl 3-methyl-4-phenyl-3,6-dihydropyridine-1(2H)-carboxylate

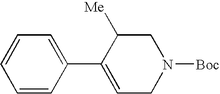

To a solution of the product of preparation 58 (3.06 g, 17.6 mmol) in acetonitrile (50 mL) and triethylamine (2.46 mL, 17.6 mmol) was added di-tert-butyl dicarbonate (4.62 g, 21 mmol) and 4-dimethylaminopyridine (108 mg, 0.9 mmol). The mixture was then stirred at room temperature for 60 h before the solvent was removed in vacuo. The residue was taken up in ethyl acetate (100 mL) and washed with 10% aqueous citric acid (100 mL) and brine (50 mL), dried (MgSO₄) and evaporated. The reside was purified by column chromatography (silica) eluting with pentane/ethyl acetate (95:5) to give the title compound as a colourless oil (3.05 g, 62%). ¹H NMR (CDCl₃, 400 MHz) δ 1.00 (d, 3H), 1.49 (s, 9H), 2.85 (br, 1H), 3.30 (dd, 1H), 3.85 (d, 2H), 4.30 (br, 1H), 5.85 (br, 1H), 7.30 (m, 5H); LRMS (ESI+) 274 [MH+].

### Preparation 60

### 5-Chloro-2-iodopyridine

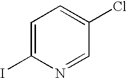

Acetyl chloride (11.05 mL, 0.155 mol) was added to a solution of 2-bromo-5-chloropyridine (20.0 g, 0.103 mol) in acetonitrile (120 mL) followed by sodium iodide (23.3 g, 0.155 mol) and the mixture was heated at reflux with a drying tube fitted for 3 hours. The reaction was cooled in an ice bath, carefully basified with saturated aqueous potassium carbonate then extracted with ethyl acetate (2×100 mL). The combined organic layers were washed with saturated aqueous sodium sulfite (200 mL), dried (MgSO₄) and evaporated. The residue was then re-submitted to identical reaction and work-up conditions in order to ensure complete reaction and this gave the title compound (18.71 g, 75%) as a brown solid. ¹H NMR (CDCl₃, 400 MHz) δ 7.30 (1H, dd), 7.65 (1H, d), 8.35 (1H, d); LRMS (APCI⁺) 240 [MH⁺].

### Preparation 61

### 5-Chloropyridine-2-carbaldehyde

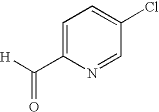

The iodide from preparation 60 (18.71 g, 78.1 mmol) was dissolved in tetrahydrofuran (100 mL) and cooled to −15° C. under nitrogen. A solution of isopropyl magnesium chloride in tetrahydrofuran (2M, 42.2 mL, 84.4 mmol) was then added dropwise, ensuring that the temperature stayed below 0° C. The reaction mixture was cooled to −15° C., stirred for 1 hour and dimethylformamide (9.0 mL, 116 mmol) was added dropwise, maintaining the temperature below 0° C. The reaction mixture was allowed to warm to room temperature and stirred for 1 hour before being re-cooled to 0° C. and carefully quenched by the dropwise addition of 2M HCl (100 mL). After the addition was complete the mixture was stirred at room temperature for 30 min before the pH was adjusted to 6-7 by the addition of saturated aqueous sodium hydrogen carbonate. The organic layer was separated and the aqueous layer was extracted with dichloromethane (2×200 mL). The combined organic layers were washed with water (200 mL), dried (MgSO₄) and concentrated on a rotary evaporator, keeping the temperature below 30° C., to give crude product (13.7 g) as a brown oil which was used without further purification. ¹H NMR (CDCl₃, 400 MHz) δ 7.35 (1H, d), 7.95 (1H, d), 8.73 (1H, s), 10.02 (1H, s); LRMS (APCI⁺) 142 [MH⁺].

### Preparation 62

### tert-Butyl (2E)-3-(5-chloropyridin-2-yl)acrylate

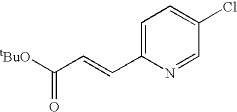

n-Butyl lithium (2.5 M in hexanes, 34 mL, 85 mmol) was added dropwise to a solution of tert-butyl diethylphosphonoacetate (19.1 mL, 81 mmol) in diethyl ether (80 mL) at −78° C. under nitrogen and stirring was continued for 30 min. A solution of the crude aldehyde from preparation 61 (from 78.1 mmol of the iodide of preparation 60) in diethyl ether (20 mL) was then added dropwise, keeping the temperature below −65° C. Once the addition was complete the mixture was allowed to warm to room temperature over 2 hours before being cautiously quenched by the addition of saturated aqueous ammonium chloride (200 mL). The mixture was extracted with diethyl ether (2×150 mL) and the combined organic extracts were washed with brine (200 mL), dried (MgSO₄) and evaporated. The residue was purified by column chromatography (silica), eluting with pentane increasing polarity to pentane/ethyl acetate 8:2, to give the title compound (13.34 g, 74% over 2 steps) as an oil. ¹H NMR (CDCl₃, 400 MHz) δ 1.51, (9H, s), 6.79 (1H, d), 7.35 (1H, d), 7.52, (1H, d), 7.66 (1H, dd), 8.55 (1H, d); LRMS (APCI⁺) 240 [MH⁺].

### Preparation 63

### (2E)-3-(5-Chloropyridin-2-yl)acrylic acid trifluoroacetic acid salt

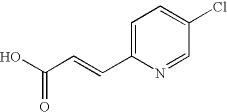

A solution of trifluoroacetic acid (10 mL) in dichloromethane (10 mL) was added dropwise to an ice cooled solution of the ester from preparation 62 (2.09 g, 8.7 mmol) in dichloromethane (10 mL) and the resulting mixture was stirred at room temperature overnight. The solvent was removed in vacuo, toluene (10 mL) was added and removed in vacuo and dichloromethane (10 mL) was added and removed in vacuo to give the title compound (2.44 g, 94%) as a red solid. ¹H NMR (CD₃OD, 400 MHz) δ 6.86 (1H, d), 7.64 (2H, m), 7.87 (1H, dd), 8.59 (1H, d); LRMS (APCI⁺) 184 [MH⁺].

### Preparation 64

### (4S)-4-Benzyl-3-[(2E)-3-(5-chloropyridin-2-yl)prop-2-enoyl]-1,3-oxazolidin-2-one

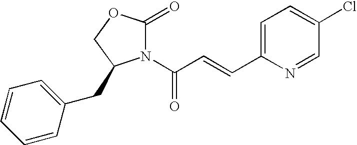

A solution of the acid from preparation 63 (2.44 g, 8.2 mmol) in tetrahydrofuran (15 mL) was cooled to −78° C. under nitrogen. Triethylamine (2.85 mL, 20 mmol) was added dropwise followed by trimethylacetyl chloride (1.11 mL, 9.0 mmol), controlling the rate of addition so that the temperature stayed below −65° C. The mixture was then stirred at −78° C. for 2 hours. ⁿBuLi (2.5 M in hexanes, 4.26 mL, 10.7 mmol) was added dropwise to a solution of (4S)-4-benzyl-1,3-oxazolidin-2-one (1.74 g, 9.8 mmol) in tetrahydrofuran (15 mL) under nitrogen at −78° C., controlling the rate of addition so that the temperature stayed below-65° C. After stirring at −78° C. for 20 minutes the solution of oxazolidinone anion was added via cannula to the mixed anhydride solution at −78° C. The reaction mixture was stirred at −78° C. for 20 minutes then allowed to warm slowly to room temperature overnight. The reaction was quenched by the addition of saturated aqueous ammonium chloride solution (30 mL) and then concentrated in vacuo to remove the tetrahydrofuran. The solid precipitate was filtered and washed with diethyl ether to give the title compound (1.52 g, 54%) as a buff solid. The ether washings were evaporated to dryness, slurried in diethyl ether and filtered to give further product (0.42 g, 15%). ¹H NMR (CDCl₃, 400 MHz) δ 2.84 (1H, t), 3.37 (1H, d), 4.22 (2H, m), 4.78 (1H, m), 7.2-7.4 (5H, m), 7.51 (1H, d), 7.69 (1H, d), 7.86 (1H, d), 8.23 (1H, d), 8.62 (1H, s); LRMS (APCI⁺) 343 [MH⁺].

### Preparation 65

### (4S)-4-Benzyl-3-{[(3S,4S)-1-benzyl-4-(5-chloropyridin-2-yl)pyrrolidin-3-yl]carbonyl}-1,3-oxazolidin-2-one

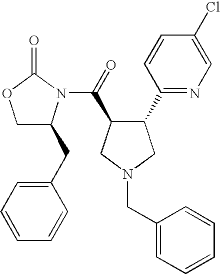

Trifluoroacetic acid (90 μL, 1.2 mmol) was added to a suspension of the oxazolidinone from preparation 64 (1.93 g, 5.6 mmol) in dichloromethane (20 mL) and N-benzyl-N-(methoxymethyl)trimethylsilylamide (2.3 mL, 9.0 mmol) was then added dropwise over 10 minutes. After the addition was complete the reaction was allowed to stir at room temperature overnight. The reaction mixture was treated with saturated aqueous sodium hydrogen carbonate solution (20 mL) and the layers were separated. The aqueous layer was extracted with dichloromethane (2×20 mL) and the combined organic layers were dried (MgSO₄) and evaporated. The residue was purified by column chromatography (silica), eluting with ethyl acetate/pentane 2:8, increasing polarity to 2:3 to give the undesired (4S)-4-benzyl-3-{[(3R,4R)-1-benzyl-4-(5-chloropyridin-2-yl)pyrrolidin-3-yl]carbonyl}-1,3-oxazolidin-2-one (1.16 g, 44%) as the first eluting component and the desired (4S)-4-benzyl-3-{[(3S,4S)-1-benzyl-4-(5-chloropyridin-2-yl)pyrrolidin-3-yl]carbonyl}-1,3-oxazolidin-2-one (1.18 g, 45%) as the second eluting component. ¹H NMR (CDCl₃, 400 MHz) δ 2.75 (2H, m), 2.92 (1H, m), 3.20 (3H, m), 3.27 (1H, br), 3.68 (2H, br), 4.14 (2H, m), 4.23 (1H, m), 4.50 (1H, m), 4.67 (1H, m), 7.10-7.40 (11H, m), 7.58 (1H, dd), 8.50 (1H, d); LRMS (APCI⁺) 476 [MH⁺].

### Preparation 66

### Methyl (3S,4S)-1-benzyl-4-(5-chloropyridin-2-yl)pyrrolidine-3-carboxylate

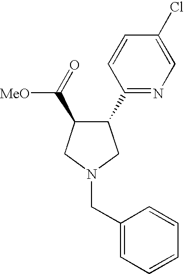

Sodium methoxide (664 mg, 12 mmol) was added to a solution of the oxazolidinone from preparation 65 (1.17 g, 2.5 mmol) and dimethyl carbonate (1.03 mL, 12 mmol) in dichloromethane (15 mL) and the reaction was stirred at room temperature overnight. The reaction mixture was concentrated in vacuo and the residue was partitioned between ethyl acetate (50 mL) and water (30 mL). The aqueous layer was neutralised by the addition of 2M HCl (˜6 mL) and then concentrated in vacuo. The residue was triturated with acetonitrile (25 mL) and then filtered. Concentration of the filtrate gave (3S,4S)-1-benzyl-4-(5-chloropyridin-2-yl)pyrrolidine-3-carboxylic acid (123 mg, 16%) as yellow solid (see preparation 8 for spectroscopic data). The ethyl acetate layer was dried (MgSO₄) and evaporated. Purification of the residue by column chromatography (silica), eluting with ethyl acetate/pentane 2:8, increasing polarity to 2:3 gave the title compound (371 mg, 45%) as a colourless oil. ¹H NMR (CDCl₃, 400 MHz) δ 2.71 (1H, t), 2.97 (1H, t), 3.05 (2H, m), 3.23 (1H, m), 3.63 (5H, m), 3.82 (1H, q), 7.15-7.35 (6H, m), 7.55 (1H, d), 8.46 (1H, s); LRMS (APCI⁺) 331 [MH⁺].

### Preparation 67

### (3S,4S)-1-Benzyl-4-(5-chloropyridin-2-yl)pyrrolidine-3-carboxylic acid bis hydrochloride

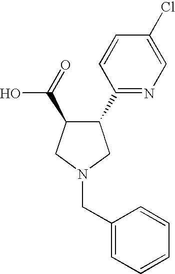

A solution of NaOH (135 mg, 3.3 mmol) in water (5 mL) was added to a solution of the ester from preparation 66 (371 mg, 1.1 mmol) in dioxane (10 mL) and the mixture was stirred at room temperature overnight. The reaction mixture was concentrated in vacuo, taken up in water (10 mL) and neutralised with 2M HCl (˜1.7 mL). The mixture was then concentrated in vacuo, triturated with acetonitrile (20 mL) and filtered. The filtrate was acidified with 2M ethereal HCl and concentrated in vacuo to give the title compound (290 mg, 68%) as a solid. ¹H NMR (CD₃OD, 400 MHz) δ 3.40-4.20 (6H, m), 4.53 (2H, m), 7.40-7.60 (6H, m), 7.81 (1H, d), 8.60 (1H, br); LRMS (APCI⁺) 317 [MH⁺].

1. A compound of formula (I): 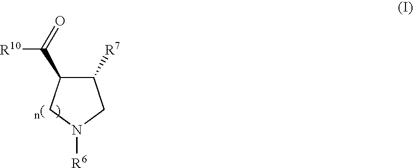
wherein:
n is 1 or 2;
R⁶ is selected from H, C₁-C₆alkyl, C₃-C₈cycloalkyl, aryl, heterocyclyl, heteroaryl, C(O)C₁-C₆alkyl, CO₂C₁-C₆alkyl, wherein each of said moieties is optionally substituted with one or more substituents independently selected from halo, CN, OH, ═O, NH₂, NHCH₃, N(CH₃)₂, C₁-C₄alkyl and C₁-C₄alkoxy;
R⁷ is selected from pyridinyl and phenyl, wherein said pyridinyl or said phenyl is optionally substituted by 1-3 groups independently selected from halo, CN, CF₃, OCF₃, OC₁-C₄alkyl and C₁-C₄alkyl;
R¹⁰ is a substituted piperidine group of formula (II): 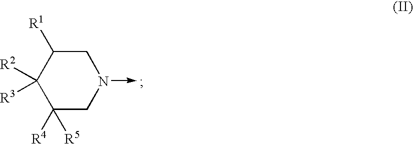
wherein
R¹ and R⁴ are each independently selected from H, C₁-C₄alkyl, OH, O(C₁-C₄alkyl), CH₂OCH₃ and NR⁸R⁹;
R² is selected from H, OH, OC₁-C₄alkyl and NR⁸R⁹;
R³ is selected from aryl or heteroaryl, wherein said moieties are optionally substituted with one or more substituents independently selected from halo, CN, CF₃, OCF₃, O(C₁-C₄alkyl), and C₁-C₄alkyl;
R⁵ is selected from H and C₁-C₄alkyl;
R⁸ is selected from H and C₁-C₄alkyl, wherein said C₁-C₄alkyl is optionally substituted with OH or OCH₃;
R⁹ is selected from H, C₁-C₄alkyl, SO₂C₁-C₄alkyl, C(O)C₁-C₄alkyl;
wherein aryl means a six or ten membered aromatic hydrocarbon ring which is optionally fused to another six or ten membered aromatic hydrocarbon ring;
wherein heteroaryl means a 5 or 6 membered aromatic ring, containing from 1 to 4 heteroatoms, said heteroatoms each independently selected from O, S and N, wherein said aromatic ring may be optionally fused to an aryl or second, non-fused, aromatic heterocyclic ring;
wherein heterocyclyl means a 4 to 7 membered saturated or partially saturated ring, containing from 1 to 2 heteroatoms each independently selected from O, S and N;
wherein halo means Cl, F, Br or I;
and pharmaceutically acceptable salts, hydrate, solvates, polymorphs and prodrugs thereof, with the provisos that:
R¹, R⁴ and R⁵ are not all simultaneously H;
when R¹ is methyl and R⁴ is H, then R⁵ is not methyl;
when R⁴ is methyl and R⁵ is H, then R¹ is not methyl; and
when R⁵ is methyl and R⁴ is H, then R¹ is not methyl.

2. A compound, salt, hydrate, solvate, polymorph or prodrug according to claim 1 wherein:
n is 1;
R¹ is selected from H, methyl, OH, OCH₃, OC₂H₅ and NR⁸R⁹;
R² is selected from H, OH, and OC₁-C₄alkyl;
R³ is selected from aryl or heteroaryl, wherein said moieties are optionally substituted with one or more substituents independently selected from halo, CN, CF₃, OCF₃, OCH₃, OC₂H₅, methyl and ethyl;
R⁴ is selected from H, methyl, OH, OCH₃, OC₂H₅ and NR⁸R⁹;
R⁵ is selected from H, methyl and ethyl;
R⁶ is selected from C₁-C₆alkyl, C₃-C₈cycloalkyl, heterocyclyl, heteroaryl, C(O)C₁-C₆alkyl, CO₂C₁-C₆alkyl, wherein each of said moieties are optionally substituted with one or more substituents independently selected from halo, CN, OH, ═O, C₁-C₄alkyl and C₁-C₄alkoxy;
R⁷ is selected from pyridinyl and phenyl, wherein said pyridinyl or said phenyl is substituted by 1-3 groups independently selected from halo, CN, CF₃, OCF₃, OCH₃ and methyl;
R⁸ is selected from H, methyl, ethyl and propyl wherein said alkyl groups is optionally substituted with OH or OCH₃;
and R⁹ is selected from H, C₁-C₄alkyl and SO₂C₁-C₄alkyl.

3. A compound, salt, hydrate, solvate, polymorph or prodrug according to claim 1 wherein R⁶ is selected from:

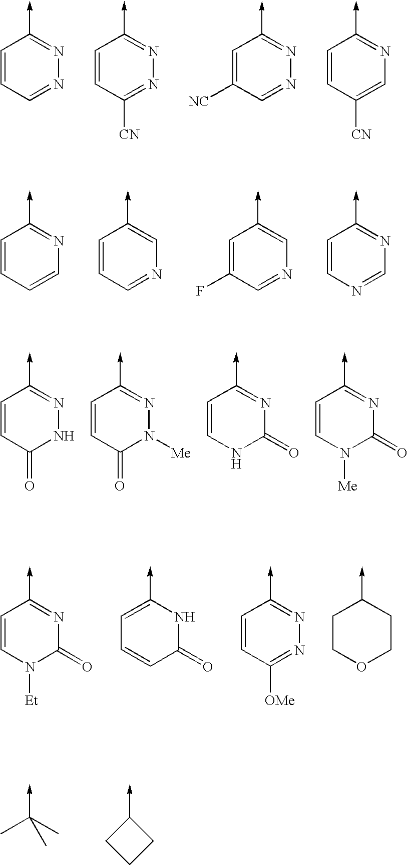

4. A compound, salt, hydrate, solvate, polymorph or prodrug according to claim 1 wherein R⁷ is selected from:

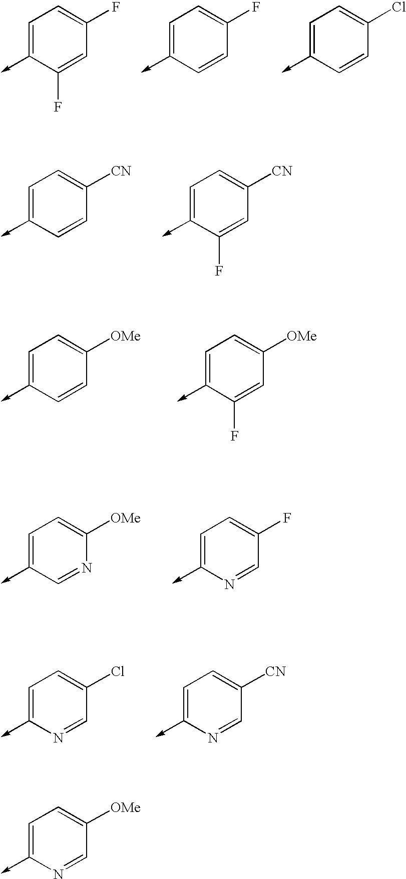

5. A compound, salt, hydrate, solvate, polymorph or prodrug according to claim 1 wherein R¹⁰ is selected from:

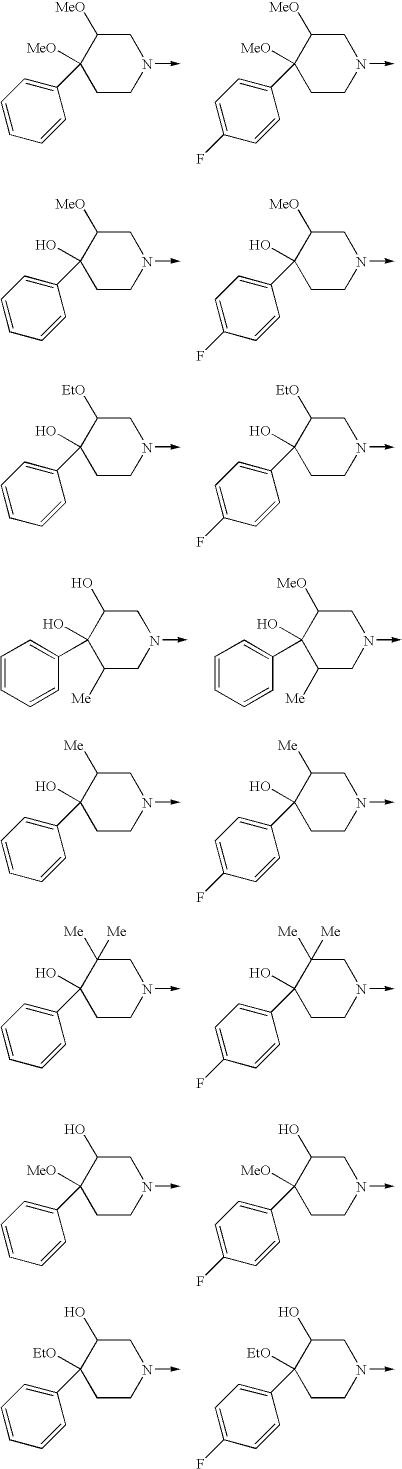

6. A compound, salt, hydrate, solvate, polymorph or prodrug according to any of claims 1 to 5 for use as a medicament.

7. A compound, salt, hydrate, solvate, polymorph or prodrug according to any of claims 1 to 5 for use as a medicament for the treatment of a condition which would benefit from the agonism of a MCR4 receptor.

8. The use of a compound, salt, hydrate, solvate, polymorph or prodrug according to any of claims 1 to 5 in the preparation of a medicament for the treatment of a condition which would benefit from the agonism of a MCR4 receptor.

9. The use according to claim 8 wherein the condition is a sexual dysfunction.

10. The use according to claim 9 wherein the sexual dysfunction is male erectile dysfunction.

11. The use according to claim 9 wherein the sexual dysfunction is female sexual arousal disorder.

12. The use according to claim 8 wherein the condition is obesity.

13. A method of treating a condition which would benefit from agonism of a MCR4 receptor which comprises administering to a patient in need thereof an effective amount of a compound, salt, hydrate, solvate, polymorph or prodrug according to any one of claims 1 to 5.

14. A pharmaceutical composition comprising a compound, salt, hydrate, solvate, polymorph or prodrug according to any one of claims 1 to 5, and a pharmaceutically acceptable diluent or carrier.
